(19) United States
(12) Patent Application Publication
(10) Pub. No.: US 2024/0123045 A1
(43) Pub. Date: Apr. 18, 2024

(54) NONAUTOLOGOUS MULTI-STRESSED CANCER CELLS AND USES THEREOF FOR VACCINATING AND TREATING CANCERS

(71) Applicant: BRENUS PHARMA, Issoire (FR)

(72) Inventors: Benoit PINTEUR, Issoire (FR); Paul BRAVETTI, Issoire (FR)

(73) Assignee: BRENUS PHARMA, Issoire (FR)

(21) Appl. No.: 18/547,760

(22) PCT Filed: Feb. 25, 2022
(86) PCT No.: PCT/EP2022/054883

§ 371 Date: Aug. 24, 2023

Related U.S. Application Data
(60) Provisional application No. 63/154,103, filed on Feb. 26, 2021.

(51) Int. Cl. A61K 39/00; C12N 5/09

(52) U.S. Cl. CPC A61K 39/0011; A61K 39/001176; C12N 5/0693; A61K 2039/5152; A61K 2039/6012; C12N 2501/06; C12N 2523/00; C12N 2529/00

(57) ABSTRACT

Advanced therapy medicinal products (AMTPs) for cell therapy. In particular, a composition including stressed HT-29, HCT-116 and LoVo cells, and immunogenic stress proteins produced by these cells in response to stresses applied in vitro. The composition allows to simultaneously counteract multiple cell resistance mechanisms observed in situ in cancer cells, and is therefore suitable for vaccinating and treating cancers in human patients.

### FIELD OF INVENTION

The present invention relates to the field of advanced therapy medicinal products (AMTPs) for cell therapy. In particular, it relates to a composition comprising stressed HT-29, HCT-116 and LoVo cells, and immunogenic stress proteins produced by these cells in response to stresses applied in vitro. The composition allows to simultaneously counteract multiple cell resistance mechanisms observed in cancer cells, and is therefore suitable for vaccinating and treating cancers in human patients.

### BACKGROUND OF INVENTION

The immune system is based on two defense mechanisms, namely, innate immunity, which is rapid but nonspecific; and acquired immunity, which is slower, but specific and has a memory. These two complementary mechanisms provide the ability to fight against external and internal “aggressions” by mobilizing the cells, either directly, during the cell-mediated immune response, or through the secretion of active molecules (e.g., immunoglobulins, cytokines, etc.) during the humoral immune response.

Immunity not only protects the host against development of primary nonviral cancers but also sculpts tumor immunogenicity. Cancer immunoediting is a process consisting of three phases: elimination (i.e., cancer immunosurveillance), equilibrium, and escape. In the first phase, the immune system fights against tumor proliferation, entailing the involvement of tissue and environmental changes that are associated with the tumor (with the mobilization of non-specific cells such as macrophage, NK cells, DC cells, etc., the secretion of anti-proliferative and/or apoptotic molecules, the production of cytokines, and the mobilization and activation of CD4⁺ and CD8⁺ T cells). During the second phase, sensitive cancer cells are eliminated and an immune selection of the most resistant cells operates. The mechanisms of resistance that are set in motion are resistance to apoptosis, secretion of inhibiting cytokines (such as TGF-β, IL-10, PGE2 or IDO), alteration of the antigen presentation (with a partial or complete loss of expression of class I major histocompatibility complex (MHC)), secretion of neutralizing molecules, and expression of MICA or MICB transcripts to counter-attack the cell-mediated immune system. During this phase, the number of cancer cells destroyed is in equilibrium with the number of resistant cells, hence the name of this phase. It corresponds inter alia to the remission phase observed during cancer-patient treatments. During the third phase, cancer cells that are resistant to the various protective mechanisms of the immune system proliferate. These cancer cells then develop a tumor mass, that is the clinical manifestation of the physiological escape phenomenon. A related escape phenomenon is also observed in advanced and metastatic stages of cancers.

Anti-cancer treatments include surgery, which objective is primarily to remove or reduce the tumor mass, but without any real inhibitory effect on the process of carcinogenesis, hence surgery is generally supplemented with various treatment therapies to fully eliminate cancer cells.

Radiation therapy is intended to bring about an alteration in the DNA of rapidly proliferating cells, which is the case with the cancer cells. The side effects of radiotherapy are twofold: first, not only cancer cells are irradiated but also healthy cells, which can cause the “cancerization” of healthy cells; second, cancer cells develop resistance to radiation-induced apoptosis by expressing chaperone proteins such as HSP, GRP, etc., resulting in an escape phenomenon.

Chemotherapies are intended to eliminate cancer cells, by acting either on the cancer cells themselves, or by inhibiting specific metabolic pathways. Chemotherapy agents include agents directly interacting with DNA such as electrophilic agents, intercalating agents, or splitting agents, agents indirectly interacting with DNA such as inhibitors of DNA synthesis (antimetabolites, topoisomerase inhibitors, inhibitors of spindle formation, etc.), neovascularization inhibitors, proteasome inhibitors, and the like. Here again, resistance mechanisms are developed by cancer cells and despite polychemotherapy strategies, a relapse phenomenon can be observed with massive tumor proliferation. Immunotherapies comprises passive immunotherapy, based on the injection of antibodies or cytokines that block a receptor, induce cell lysis, stimulate cytotoxicity, or lift the inhibition of apoptosis; and active immunotherapy, also called “immunization by vaccination”, in which patients are immunized against cancer using “advanced therapy medicinal products” (ATMPs), a new class of medicines defined in Europe by EU Regulation 1394/2007.

A wide variety of ATMPs for gene therapy (called GTMPs) have been used in clinical trials in a wide variety of cancers; as few examples, adenoviral vectors were used to express p53 in head and neck carcinoma or to express CD40L in bladder carcinoma; oncolytic herpes virus encoding GM-CSF was used in patients with melanoma; and T cells engineered with chimeric antigen receptors (CARs) redirected against various tumor-associated antigens have also been recently used.

AMTPs for cell therapy (called CTMPs) have also been used, including autologous dendritic cells (DCs) loaded with tumor antigens, in the attempt to elicit clinically relevant immune responses. For example, the FDA and EMA approved in 2010 and 2013, respectively, a DC-based therapeutic vaccine for prostate cancer (Sipuleucel-T, trade name “Provenge”, developed by Dendreon Pharmaceuticals, LLC)—although the European Commission withdrew the marketing authorization for Sipuleucel-T in the European Union in 2015.

However, with AMTPs as well, escape phenomena are observed, that are similar to the natural escape phenomena described in 3-phase cancer immunoediting involving the immune system.

There is therefore still a need for therapeutic strategies for the treatment of cancer, which can provide an inherent effectiveness and/or contribute to a multitherapeutic strategy.

A number of studies have been carried out to better understand cancer resistance phenomena, and in particular the role of chaperone proteins in the resistance to apoptosis.

In particular, heat shock proteins (HSPs), glucose regulated proteins (GRPs) and multidrug resistance proteins (MRP) are known to be resistance factors and to have a protective effect. These proteins are produced by cancer cells as a mechanism of resistance, as a result of metabolic attacks such as hypoxia, low carbohydrate concentration, etc., of physical attacks such as thermal stress, radiations, etc., or of chemical attacks such as medication.

As a consequence, studies have been carried out to develop treatment strategies based on inhibition of these chaperone proteins, but these are not without side effects, including a high toxicity level for overall mixed results. Studies have also been carried out to use chaperone proteins, in particular HSP70, as factors of immunization against cancer cells in mice. This approach however focuses on a specific chaperone protein and does not take into account the entire set of protection mechanisms developed by cancer cells.

To address this issue, the Inventors have described an in vitro process for obtaining pharmaceutical or vaccine compositions capable of counteracting cancer cell resistance mechanisms, a process which is otherwise adapted to the in vitro production of autologous cancer cells with particular resistance mechanism[s] identical to that developed in situ by cancer cells subjected in vivo to a specific stress (or stresses) in the course of a treatment protocol applied to a patient (U.S. Pat. No. 11,096,995; European Pat. 3 057 981). Following administration of these compositions in mice in vivo, data showed a favorable impact on tumor volume and weight, validating the proof of concept.

Here, the Inventors go further, and provide with a new composition comprising a selection of nonautologous multi-stressed cells, simultaneously counteracting multiple resistance mechanisms, suitable for vaccinating and treating various cancer in human patients.

### SUMMARY

The present invention is as disclosed hereafter, and in particular in the appended claims.

In particular, the present invention relates to a composition comprising (i) stressed HT-29, HCT-116 and LoVo cells, and (ii) immunogenic stress proteins produced by these cells in response to a stress applied in vitro.

In one embodiment, stressed HT-29, HCT-116 and LoVo cells have developed resistance mechanism in response to one or several stress[es] applied in vitro, selected from the group comprising radiations, thermal stress, chemical stress, metabolic stress and any combinations thereof, leading to the production of the stress proteins.

In one embodiment, stressed HT-29, HCT-116 and LoVo cells are non-proliferative.

In one embodiment, immunogenic stress proteins are haptenated. In one embodiment, immunogenic stress proteins are haptenated with an hapten selected from the group comprising 2,4-dinitrophenyl (DNP); 2,4-dinitrofluorobenzene; sulfanilic acid; N-iodoacetyl-N′-(5-sulfonic-naphthyl)ethylene diamine; anilin; p-amino benzoic acid; biotin; fluorescein and derivatives thereof (including FITC, TAMRA, and Texas Red); digoxigenin; 5-nitro-3-pyrazolecarbamide; 4,5-dimethoxy-2-nitrocinnamide; 2-(3,4-dimethoxyphenyl)-quinoline-4-carbamide; 2,1,3-benzoxadiazole-5-carbamide; 3-hydroxy-2-quinoxalinecarbamide, 4-(dimethylamino)azobenzene-4′-sulfonamide (DABSYL); rotenone isooxazolinI(E)-2-(2-(2-oxo-2,3-dihydro-1H-benzo[b][1,4]diazepin-4-yl)phenozy)aceta mide; 7-(diethylamino)-2-oxo-2H-chromene-3-carboxylic acid; 2-acetamido-4-methyl-5-thiazolesulfonamide; and p-methoxyphenylpyrazopodophyllamide. In one embodiment, immunogenic stress proteins are haptenated with 2,4-dinitrophenyl (DNP).

In one embodiment, the composition is a pharmaceutical composition or a vaccine composition, and further comprises at least one pharmaceutically acceptable excipient.

In one embodiment, the composition comprises from about 10⁵ to about 10⁸ stressed HT-29, HCT-116 and LoVo cells.

The present invention further relates to this composition, for use in treating cancer in a subject in need thereof. It also relates to a method of treating cancer in a subject in need thereof, comprising administering this composition to the subject.

The present invention further relates to an intermediate composition comprising (i) one of stressed HT-29 cells, stressed HCT-116 cells and stressed LoVo cells, and (ii) stress proteins, wherein the one of stressed HT-29 cells, stressed HCT-116 cells or stressed LoVo cells have developed resistance mechanism in response to (i) a metabolic stress, (ii) radiations and (iii) a thermal stress applied in vitro, leading to the production of the stress proteins.

The present invention further relates to an intermediate composition comprising (i) one of stressed HT-29 cells, stressed HCT-116 cells and stressed LoVo cells, and (ii) stress proteins, wherein the one of stressed HT-29 cells, stressed HCT-116 cells or stressed LoVo cells have developed resistance mechanism in response to (i) a metabolic stress, and (ii) a chemical stress applied in vitro, leading to the production of the stress proteins.

The present invention further relates to a method of manufacturing said intermediate compositions, comprising the following steps:
a) cultivating HT-29, HCT-116 or LoVo cells in a suitable culture medium;
b) subjecting the HT-29, HCT-116 or LoVo cells cultured in step a) to one or several stress[es] in vitro, wherein these HT-29, HCT-116 or LoVo cells develop resistance mechanisms in response to the one or several stress[es] and thereby produce stress proteins,
c) recovering the stressed HT-29, HCT-116 or LoVo cells together with the stress proteins they have produced in step b), and
d) treating the stressed HT-29, HCT-116 or LoVo cells and the stress proteins they have produced, all together recovered in step c), with a molecule or by a process capable of rendering the stress proteins immunogenic.

In one embodiment, step c) is carried out at least several hours after completion of step b), preferably at least 12 hours or more after completion of step b).

In one embodiment, step d) comprises linking the stress proteins to or complexing the stress proteins with a means capable to confer immunogenicity. In one embodiment, the means capable to confer immunogenicity is an hapten. In one embodiment, the means capable to confer immunogenicity is an hapten selected from the group comprising 2,4-dinitrophenyl (DNP); 2,4-dinitrofluorobenzene; sulfanilic acid; N-iodoacetyl-N′-(5-sulfonic-naphthyl)ethylene diamine; anilin; p-amino benzoic acid; biotin; fluorescein and derivatives thereof (including FITC, TAMRA, and Texas Red); digoxigenin; 5-nitro-3-pyrazolecarbamide; 4,5-dimethoxy-2-nitrocinnamide; 2-(3,4-dimethoxyphenyl)-quinoline-4-carbamide; 2,1,3-benzoxadiazole-5-carbamide; 3-hydroxy-2-quinoxalinecarbamide, 4-(dimethylamino)azobenzene-4′-sulfonamide (DABSYL); rotenone isooxazoline; (E)-2-(2-(2-oxo-2,3-dihydro-1H-benzo[b][1,4]diazepin-4-yl)phenozy)acetamide; 7-(diethylamino)-2-oxo-2H-chromene-3-carboxylic acid; 2-acetamido-4-methyl-5-thiazolesulfonamide; and p-methoxyphenylpyrazopodophyllamide. In one embodiment, the means capable to confer immunogenicity is 2,4-dinitrophenyl (DNP).

In one embodiment—where the method is for manufacturing an intermediate composition “DS-A” comprising (i) one of stressed HT-29 cells, stressed HCT-116 cells and stressed LoVo cells, and (ii) stress proteins, wherein the one of stressed HT-29 cells, stressed HCT-116 cells or stressed LoVo cells have developed resistance mechanism in response to (i) a metabolic stress, (ii) radiations and (iii) a thermal stress applied in vitro, leading to the production of the stress proteins—, step b) of said method comprises subjecting the HT-29, HCT-116 or LoVo cells cultured in step a) to the following stresses in vitro, applied concomitantly or successively:
(i) an in vitro culture in a depleted medium, under hypoxia, and/or at low pH; preferably an in vitro culture in low serum culture conditions in a 2% FBS culture medium,
(ii) an in vitro radiation with a total dose ranging from about 0.25 to about 25 Gy, preferably from about 1 to about 15 Gy, preferably for a period ranging from about 1 to about 20 minutes; preferably an in vitro radiation with a total dose of 10 Gy for a period of about 1 to about 5 minutes, and
(iii) an in vitro thermic choc at a temperature ranging from about 38° C. to about 45° C., applied to the cells for a period ranging from about 15 minutes to about 4 hours; preferably an in vitro thermic choc at a temperature of about 42° C. for a period of about 60 minutes.

In one embodiment—where the method is for manufacturing an intermediate composition “DS-B” comprising (i) one of stressed HT-29 cells, stressed HCT-116 cells and stressed LoVo cells, and (ii) stress proteins, wherein the one of stressed HT-29 cells, stressed HCT-116 cells or stressed LoVo cells have developed resistance mechanism in response to (i) a metabolic stress, and (ii) a chemical stress applied in vitro, leading to the production of the stress proteins—step b) of said method comprises subjecting the HT-29, HCT-116 or LoVo cells cultured in step a) to the following stresses in vitro, applied concomitantly or successively:
(i) an in vitro culture in a depleted medium, under hypoxia, and/or at low pH; preferably an in vitro culture in low serum culture conditions in a 2% FBS culture medium,
(ii) an in vitro exposition to at least one or several chemotherapeutic agents and/or alcohols, for a period ranging from about 6 hours to about 120 hours.

According to the latest embodiment, the in vitro exposition to at least one or several chemotherapeutic agents and/or alcohols may be as follows:
when the cells are HT-29 cells, the in vitro exposition at (ii) is to about 13 μM oxaliplatin for a period of about 72 hours; or
when the cells are HCT-116 cells, the in vitro exposition at (ii) is to about 315 nM SN-38 (7-ethyl-10-hydroxy-camptothecin) for a period of about 48 hours; or
when the cells are LoVo cells, the in vitro exposition at (ii) is to about 5 μM fluorouracil (5-FU) for a period of about 48 hours.

The present invention further relates to a method of manufacturing the composition of the invention, comprising the following steps:
a. obtaining six intermediate compositions, wherein the six intermediate compositions are:
   1) an intermediate composition “DS-A” comprising stressed HT-29 cells and stress proteins,
   2) an intermediate composition “DS-A” comprising stressed HCT-116 cells and stress proteins,
   3) an intermediate composition “DS-A” comprising stressed LoVo cells and stress proteins,
   4) an intermediate composition “DS-B” comprising stressed HT-29 cells and stress proteins,
   5) an intermediate composition “DS-B” comprising stressed HCT-116 cells and stress proteins,
   6) an intermediate composition “DS-B” comprising stressed LoVo cells and stress proteins,
b. mixing these six intermediate compositions together.

In one embodiment, the six intermediate compositions are mixed together in an equal ratio of stressed HT-29, HCT-116 and LoVo cells.

### Definitions

In the present invention, the following terms have the following meanings:

“HT-29 cells”, as used herein, refers to a human colon adenocarcinoma cell line isolated from a primary tumor in 1964 from a 44-year-old woman. It comprises, inter alia, the following sequence variations:
heterozygous for APC p.Glu853Ter (c.2557G>T),
heterozygous for APC p.Thr1556Asnfs*3 (c.4666dupA),
heterozygous for BRAF p.Val600Glu (c.1799T>A),
heterozygous for PIK3CA p.Pro449Thr (c.1345C>A),
homozygous for SMAD4 p.Gln311Ter (c.931C>T), and
homozygous for TP53 p.Arg273His (c.818G>A).

This cell line is referenced under accession number “CVCL_0320” on the Cellosaurus database and “HTB-38™” in the ATCC repository collection, from which it is commercially available. Other accession numbers for this cell line include “0111” in the Banco Cdlulas do Rio de Janeiro (BCRJ); “ACC 299” in the Leibniz Institute DSMZ-German Collection of Microorganisms and Cell Cultures GmbH; “91072201” in the General Collection of the European Collection of Authenticated Cell Cultures (ECACC); “30038” in the Korean Cell Line Bank (KCLB); and “GDC0149” in the China Center for Type Culture Collection (CCTCC). The content of these collections, in particular the description of the genotype, HLA typing, STR profile and phenotype of this cell line, is hereby incorporated by reference.

“HCT-116 cells”, as used herein, refers to a human colon carcinoma cell line isolated from a primary tumor in 1981 from a 48-year-old man. It comprises, inter alia, the following sequence variations:
homozygous for ACVR2A p.Lys437Argfs*5 (c.1310delA),
heterozygous for BRCA2 p.Ile2675Aspfs*6 (c.8021dupA),
heterozygous for CDKN2A p.Arg24Serfs*20 (c.68dupG),
heterozygous for CDKN2A p.Asp74fs*21 (c.220delG),
heterozygous for CDKN2A p.Glu33Argfs*20 (c.97delG),
heterozygous for CTNNB1 p.Ser45del (c.133_135delTCT),
heterozygous for EP300 p.Met1470Cysfs*22 (c.4408delA),
heterozygous for EP300 p.Asn1700Thrfs*9 (c.5099delA),
heterozygous for KRAS p.Gly13Asp (c.38G>A),
heterozygous for PIK3CA p.His1047Arg (c.3140A>G), and
homozygous for TGFBR2 p.Lys128Serfs*35 (c.383delA).

This cell line is referenced under accession number “CVCL_0291” on the Cellosaurus database and “CCL-247™” in the ATCC repository collection, from which it is commercially available. Other accession numbers for this cell line include “0288” in the Banco Cdlulas do Rio de Janeiro (BCRJ); “ACC 581” in the Leibniz Institute DSMZ-German Collection of Microorganisms and Cell Cultures GmbH; “91091005” in the General Collection of the European Collection of Authenticated Cell Cultures (ECACC); and “10247” in the Korean Cell Line Bank (KCLB). The content of these collections, in particular the description of the genotype, HLA typing, STR profile and phenotype of this cell line, is hereby incorporated by reference.

“LoVo cells”, as used herein, refers to a human colorectal adenocarcinoma cell line isolated from a fragment of a metastatic tumor nodule in the left supraclavicular region in 1971 from a 56-year-old man. It comprises, inter alia, the following sequence variations:
heterozygous for ACVR2A p.Lys437Argfs*5 (c.1310delA),
heterozygous for APC p.Arg1114Ter (c.3340C>T),
heterozygous for APC p.Met1431fs*42 (c.4289delC),
heterozygous for APC p.Arg2816Gln (c.8447G>A),
heterozygous for B2M p.Leu15Phefs*41 (c.43_44delCT),
heterozygous for FBXW7 p.Arg505Cys (c.1513C>T),
heterozygous for KRAS p.Gly13Asp (c.38G>A),
heterozygous for SMAD2 p.Ala292Val (c.875C>T), and
homozygous for TGFBR2 p.Lys128Serfs*35 (c.383delA).

This cell line is referenced under accession number “CVCL_0399” on the Cellosaurus database and “CCL-229™” in the ATCC repository collection, from which it is commercially available. Other accession numbers for this cell line include “0332” in the Banco Cdlulas do Rio de Janeiro (BCRJ); “ACC 350” in the Leibniz Institute DSMZ-German Collection of Microorganisms and Cell Cultures GmbH; “87060101” in the General Collection of the European Collection of Authenticated Cell Cultures (ECACC); and “10229” in the Korean Cell Line Bank (KCLB). The content of these collections, in particular the description of the genotype, HLA typing, STR profile and phenotype of this cell line, is hereby incorporated by reference

“Chemotherapeutic agent”, as used herein, refers to any molecule that is effective in inhibiting tumor growth. Examples of chemotherapeutic agents include those described under subgroup L01 of the Anatomical Therapeutic Chemical (ATC) Classification System. Further examples of chemotherapeutic agents include, but are not limited to:
i. alkylating agents, such as, e.g.:
   nitrogen mustards, including chlormethine, cyclophosphamide, ifosfamide, trofosfamide, chlorambucil, melphalan, prednimustine, bendamustine, uramustine, chlornaphazine, cholophosphamide, estrarnustine, mechlorethamine, mechlorethamine oxide hydrochloride, novembichin, phenesterine, uracil mustard and the like;
   nitrosoureas, including carmustine, lomustine, semustine, fotemustine, nimustine, ranimustine, streptozocin, chlorozotocin, and the like;
   alkyl sulfonates, including busulfan, mannosulfan, treosulfan, and the like;
   aziridines, including carboquone, thiotepa, triaziquone, triethylenemelamine, benzodopa, meturedopa, uredopa, and the like; hydrazines, including procarbazine, and the like;
   triazenes, including dacarbazine, temozoloinide, and the like; ethylenimines and methylamelamines, including altretamine, triethylenemelainine, trietylenephosphoramide, triethylenethiophosphaorarnide, trimethylolomelamine and the like;
   and others, including mitobronitol, pipobroman, actinomycin, bleomycin, mitomycins (including mitomycin C, and the like), plicamycin, and the like;
ii. acetogenins, such as, e.g., bullatacin, bullatacinone, and the like;
iii. benzodiazepines, such as, e.g., 2-oxoquazepam, 3-hydroxyphenazepam, bromazepam, camazepam, carburazepam, chlordiazepoxide, cinazepam, cinolazepam, clonazepam, cloniprazepam, clorazepate, cyprazepam, delorazepam, demoxepam, desmethylflunitrazepam, devazepide, diazepam, diclazepam, difludiazepam, doxefazepam, elfazepam, ethyl carfluzepate, ethyl dirazepate, ethyl loflazepate, flubromazepam, fletazepam, fludiazepan, flunitrazepain, flurazepam, flutemazepam, flutoprazepam, fosazepam, gidazepam, halazepam, iclazepam, irazepine, kenazepine, ketazolam, lorazepam, lormetazepam, lufuradom, meclonazepam, medazepam, menitrazepam, inetaclazepam, motrazepam, N-desalkylflurazepam, nifoxipam, nimetazepam, nitemazepam, nitrazepam, nitrazepate, nordazepam, nortetrazepam, oxazepam, phenazepam, pinazepam, pivoxazepam, prazepam, proflazepam, quazepan. QI-II-66, reclazepam, R4491533, Ro5-4864, SH-I-048A, sulazepam, temazepam, tetrazepam, tifluadom, tolufazepam, triflunordazepam, tuclazepam, uldazepam, arfendazam, clobazam, CP-1414S, lofendazam, triflubazam, girisopam, GYKI-52466. GYKI-52895, nerisopam, talampanel, tofisopam, adinazolam, alprazolam, bromazolam, clonazolain, estazolam, flualprazolam, flubromazolam, flunitrazolam, nitrazolam, pyrazolam, triazolam, bretazenil, climazolam, EVrT-201, FG-8205, flumazenil, GL-II-73, imidazenil, ¹²³I-iomazenil. L-655,708, loprazolam, midazolam, PWZ-029, remimazolam, Rol5-4513, Ro48-6791, Ro48-8684, Ro4938581, sarmazenil, SH-053-R-CH3.2′F, cloxazolam, flutazolam, haloxazolam, mexazolam, oxazolam, bentazepam, clotiazepam, brotizolam, ciclotizolam, deschloroetizolam, etizolam, fluclotizolam, israpafant, JQI, metizolam, olanzapine, telenzepine, lopirazepam, zapizolam, razobazam, ripazepam, zolazepam, zomebazam, zometapine, premazepam, clazolam, anthramycin, avizafone, rilmazafone, and the like;
iv. antimetabolites, such as, e.g.:
   antifolates. including aminopterin, methotrexate, pemetrexed, pralatrexate, pteropterin, raltitrexed, denopterin, trimetrexate, pemetrexed, and the like;
   purine analogues, including pentostatin, cladribine, clofarabine, tludarabine, nelarabine, tioguanine, mercaptopurine, and the like;
   pyrimidine analogues, including fluorouracil (5-FU), capecitabine, doxifluridine, tegafur, tegafur/gimeracil/oteracil, carmofur, floxuridine, cytarabine, gemcitabine, azacytidine, decitabine, and the like; and
   hydroxycarbamide;
v. androgens, such as, e.g., calusterone, dromostanolone propionate, epitiostanol, mepitiostane, testolactone, and the like;
vi. anti-adrenals, such as, e.g., aminoglutethimide, mitotane, trilostane, and the like;
vii. folic acid replenishers, such as, e.g., frolinic acid, and the like;
viii. maytansinoids, such as, e.g., maytansine, ansamitocins, and the like;
ix. platinum analogs, such as, e.g., platinum, carboplatin, cisplatin, dicycloplatin, nedaplatin, oxaliplatin, satraplatin, and the like;
x. antihormonal agents, such as, e.g.:
   anti-estrogens, including tamoxifen, raloxifene, aromatase inhibiting 4(5)-imidazoles, 4-hydroxytamoxifen, trioxifene, keoxifene, LY117018, onapristone, toremifene, and the like;
   anti-androgens, including flutamide, nilutamide, bicalutamide, leuprolide, goserelin, and the like;
xi. trichothecenes, such as, e.g., T-2 toxin, verracurin A, roridinA, anguidine and the like;
xii. toxoids, such as, e.g., cabazitaxel, docetaxel, larotaxel, ortataxel, paclitaxel, tesetaxel, and the like;
xiii. others, such as, e.g., camptothecin (including its derivatives: belotecan, cositecan, etirinotecan, pegol, exatecan, gimatecan, irinotecan, lurtotecan, rubitecan, silatecan, SN-38 (7-ethyl-10-hydroxy-camptothecin), and topotecan); bryostatin: callystatin; CC-1065 (including its adozelesin, carzelesin and bizelesin synthetic analogues); cryptophycins (including cryptophycin 1 and cryptophycin 8): dolastatin; duocarmycin (including its synthetic analogues: KW-2189 and CBI-TMI); eleutherobin; pancratistatin; sarcodictyin; spongistatin; aclacinomysins; authramycin; azaserine; bleonycin; cactinomycin; carabicin; canninomycin; carzinophilin: chromomycins; dactinomycin; daunorubicin; detorubicin; 6-diazo-5-oxo-L-norleucine; doxorubicin (including morpholino-doxorubicin, cyanomorpholino-doxorubicin, 2-pyrrolino-doxorubicin, deoxydoxorubicin, and the like); epirubicin; esorubicin: idanrbicin; narcellomycin: mycophenolic acid; nogalarnycin; olivomycins; peplomycin; poteiromycin; puromycin; quelamycin; rodorubicin: streptomgrin; streptozocin; tubercidin; ubenimex; zinostatin; zorubicin: aceglatone; aldophospharnide glycoside; aminolevulinic acid; amsacrine; bestrabucil; bisantrene: edatraxate: defofamine; demecolcine; diaziquone; elfornithine; elliptinium acetate; epothilone; etoglucid; gallium nitrate; hydroxyurea; lentinan; lonidamine: mitoguazone; mitoxantrone; mopidamol: nitracrine: phenamet; pirarubicin; podophyllinic acid; 2-ethylhydrazide: PSK®; razoxane; rhizoxin; sizofiran; spirogennanium; tenuazonic acid; 2,2′2′″-trichlorotriethylarnine; urethan; vindesine; dacarbazine; mannomustine; mitobromtol; mitolactol; pipobroman; gacytosine; arabinoside; 6-thioguanine; vinblastine; etoposide; vincristine; vinorelbine; navelbine; novantrone; teniposide; daunomycin; xekida: ibandronate: CPT-11: topoisomerase inhibitor RFS 2000; topoisomerase I inhibitor SN38; difluoromethylornithine; retinoic acid; and the like.

“Detectable levels”, as used herein in the context of a protein, in particular of a stress protein, means that said protein is present in the composition at stake in an amount or concentration that can be detected by means and methods classically used by the skilled artisan to detect proteins. Such means and methods are well-known in the art, and include, without limitation, mass spectrometry, such as liquid chromatography coupled to tandem mass spectrometry (LC-MS/MS), as detailed in Examples 3-5 below.

“Hapten”, as used herein, refers to a small molecule that elicits an immune response only when attached to a large carrier such as a protein. Once the patient has generated antibodies to a hapten-carrier conjugate, the hapten may be able to bind to the antibodies, but it will usually not initiate an immune response; usually, only the hapten-carrier conjugate can do this. Examples of haptens include, but are not limited to, 2,4-dinitrophenyl (DNP); 2,4-dinitrofluorobenzene; sulfanilic acid; N-iodoacetyl-N′-(5-sulfonic-naphthyl)ethylene diamine; anilin; p-amino benzoic acid; biotin; fluorescein and derivatives thereof (including FITC, TAMRA, and Texas Red); digoxigenin; 5-nitro-3-pyrazolecarbamide; 4,5-dimethoxy-2-nitrocinnamide; 2-(3,4-dimethoxyphenyl)-quinoline-4-carbamide; 2,1,3-benzoxadiazole-5-carbamide; 3-hydroxy-2-quinoxalinecarbamide, 4-(dimethylamino)azobenzene-4′-sulfonamide (DABSYL); rotenone isooxazoline; (E)-2-(2-(2-oxo-2,3-dihydro-1H-benzo[b][1,4]diazepin-4-yl)phenozy)acetamide; 7-(diethylamino)-2-oxo-2H-chromene-3-carboxylic acid; 2-acetamido-4-methyl-5-thiazolesulfonamide; and p-methoxyphenylpyrazopodophyllamide.

“Overexpression”, and declinations thereof, refers herein to a relative or absolute expression and/or abundance of a marker or protein which is at least 10% higher, preferably at least 15%, 20%, 25%, 30%, 35%, 40%, 45%, 50% or more higher in a subject cell or subject cell population, as compared to a reference cell or reference cell population. Alternatively or additionally, overexpression may also refer to a mean fluorescence intensity (MFI) which is at least 10% higher, preferably at least 15%, 20%, 25%, 30%, 35%, 40%, 45%, 50% or more higher in a subject cell or subject cell population, as compared to a reference cell or reference cell population. In particular, the reference cell is a cell (or the reference cell population is a population comprising or preferably consisting of those cells) from the same cell line as the subject cell, but which has not been subject to the method of manufacturing the composition or intermediate compositions described herein; in particular a cell (or a population comprising or preferably consisting of those cells) from the same cell line as the subject cell, which is cultured in classical conditions such as, e.g., in 10% FBS, and is not subjected to any of a metabolic stress, radiations, a thermal stress and/or a chemical stress applied in vitro, as described hereafter; or alternatively that is cultured in a depleted medium (e.g., partially or totally lacking one or several substances that are useful or even essential for the growth and/or survival of cancer cells), under hypoxia, or at low pH (e.g., below pH 6.5) (i.e., subjected to a metabolic stress), but not subjected to any other stress among radiations, a thermal stress and/or a chemical stress applied in vitro, as described hereafter.

“Underexpression”, and declinations thereof, refers herein to a relative or absolute expression and/or abundance of a marker or protein which is at least 10% lower, preferably at least 15%, 20%, 25%, 30%, 35%, 40%, 45%, 50% or more lower in a subject cell or subject cell population, as compared to a reference cell or reference cell population. Alternatively or additionally, overexpression may also refer to a mean fluorescence intensity (MFI) which is at least 10% lower, preferably at least 15%, 20%, 25%, 30%, 35%, 40%, 45%, 50% or more lower in a subject cell or subject cell population, as compared to a reference cell or reference cell population. In particular, the reference cell (or the reference cell population is a population comprising or preferably consisting of those cells) is a cell from the same cell line as the subject cell, but which has not been subject to the method of manufacturing the composition or intermediate compositions described herein; in particular a cell (or a population comprising or preferably consisting of those cells) from the same cell line as the subject cell, which is cultured in classical conditions such as, e.g., in 10% FBS, and is not subjected to any of a metabolic stress, radiations, a thermal stress and/or a chemical stress applied in vitro, as described hereafter; or alternatively that is cultured in a depleted medium (e.g., partially or totally lacking one or several substances that are useful or even essential for the growth and/or survival of cancer cells), under hypoxia, or at low pH (e.g., below pH 6.5) (i.e., subjected to a metabolic stress), but not subjected to any other stress among radiations, a thermal stress and/or a chemical stress applied in vitro, as described hereafter.

“Vaccine composition”, as used herein, refers to a composition which comprises at least one antigen or immunogen (such as, e.g., immunogenic stress and/or resistance proteins) and optionally an adjuvant, in a pharmaceutically acceptable excipient, and which is useful for inducing an immune response in a patient upon administration.

“Adjuvant”, as used herein, refers to a substance that enhances, increases and/or potentiates an immune response to an antigen (such as, e.g., immunogenic stress and/or resistance proteins) in a subject upon administration.

“Pharmaceutically acceptable excipient”, as used herein, refers to a solid, semi-solid or liquid component of a pharmaceutical composition or a vaccine composition that is not an active ingredient (i.e., that is neither the stressed HT-29, HCT-116 and LoVo cells nor the immunogenic stress and/or resistance proteins), and that does not produce an adverse, allergic or other untoward reaction when administered to a patient. The most of these pharmaceutically acceptable excipients are described in detail in, e.g., Allen (Ed.), 2017. Ansel's pharmaceutical dosage forms and drug delivery systems (11^{th} ed.). Philadelphia, PA: Wolters Kluwer; Remington, Allen & Adeboye (Eds.), 2013. Remington: The science and practice of pharmacy (22^{nd} ed.). London: Pharmaceutical Press; and Sheskey, Cook & Cable (Eds.), 2017. Handbook of pharmaceutical excipients (8^{th} ed.). London: Pharmaceutical Press.

### DETAILED DESCRIPTION

### Drug Product (DP/STC-1010)

The present invention relates to a composition comprising or consisting of (i) at least one of stressed HT-29, HCT-116 or LoVo cells, and (ii) immunogenic stress and/or resistance proteins, wherein the stress and/or resistance proteins were produced by these at least one of HT-29, HCT-116 or LoVo cells in response to a stress that was applied in vitro.

In one embodiment, the composition comprises or consists of (i) stressed HT-29 cells, and (ii) immunogenic stress and/or resistance proteins, wherein the stress and/or resistance proteins were produced by these HT-29 cells in response to a stress that was applied in vitro.

In one embodiment, the composition comprises or consists of (i) stressed HCT-116 cells, and (ii) immunogenic stress and/or resistance proteins, wherein the stress and/or resistance proteins were produced by these HCT-116 cells in response to a stress that was applied in vitro.

In one embodiment, the composition comprises or consists of (i) stressed LoVo cells, and (ii) immunogenic stress and/or resistance proteins, wherein the stress and/or resistance proteins were produced by these LoVo cells in response to a stress that was applied in vitro.

In one embodiment, the composition comprises or consists of (i) stressed HT-29 and HCT-116 cells, and (ii) immunogenic stress and/or resistance proteins, wherein the stress and/or resistance proteins were produced by these HT-29 and HCT-116 cells in response to a stress that was applied in vitro.

In one embodiment, the composition comprises or consists of (i) stressed HT-29 and LoVo cells, and (ii) immunogenic stress and/or resistance proteins, wherein the stress and/or resistance proteins were produced by these HT-29 and LoVo cells in response to a stress that was applied in vitro.

In one embodiment, the composition comprises or consists of (i) stressed HCT-116 and LoVo cells, and (ii) immunogenic stress and/or resistance proteins, wherein the stress and/or resistance proteins were produced by these HCT-116 and LoVo cells in response to a stress that was applied in vitro.

In one embodiment, the composition comprises or consists of (i) stressed HT-29, HCT-116 and LoVo cells, and (ii) immunogenic stress and/or resistance proteins, wherein the stress and/or resistance proteins were produced by these HT-29, HCT-116 and LoVo cells in response to a stress that was applied in vitro.

In the following, any reference to the composition comprises HT-29, HCT-116 and LoVo cells is intended to encompass compositions comprising one, two or the three of HT-29, HCT-116 and LoVo cells.

According to the invention, the composition comprises HT-29, HCT-116 and LoVo cells which are stressed. By “stressed”, it is meant that these cells have developed [a] resistance mechanism[s] in response to [a] stress[es] applied in vitro. As a consequence, these cells have produced stress and/or resistance proteins which form part of the composition.

In one embodiment, the stress is selected from the group comprising or consisting of radiations, thermal stress, chemical stress, metabolic stress and any combinations thereof.

In one embodiment, the stress includes a combination, whether concomitant or successive, of two, three or more of radiations, thermal stress, chemical stress, and metabolic stress.

In one embodiment, the composition of the invention comprises or consists of:
stressed HT-29 cells and stress and/or resistance proteins produced by these HT-29 cells in response to (i) a metabolic stress, (ii) radiations and (iii) a thermal stress applied in vitro;
stressed HCT-116 cells and stress and/or resistance proteins produced by these HCT-116 cells in response to (i) a metabolic stress, (ii) radiations and (iii) a thermal stress applied in vitro; and
stressed LoVo cells and stress and/or resistance proteins produced by these LoVo cells in response to (i) a metabolic stress, (ii) radiations and (iii) a thermal stress applied in vitro.

In one embodiment, the composition of the invention comprises or consists of:
stressed HT-29 cells and stress and/or resistance proteins produced by these HT-29 cells in response to (i) a metabolic stress, and (ii) a chemical stress applied in vitro;
stressed HCT-116 cells and stress and/or resistance proteins produced by these HCT-116 cells in response to (i) a metabolic stress, and (ii) a chemical stress applied in vitro; and
stressed LoVo cells and stress and/or resistance proteins produced by these LoVo cells in response to (i) a metabolic stress, and (ii) a chemical stress applied in vitro.

In one embodiment, the composition of the invention comprises or consists of:
stressed HT-29 cells and stress and/or resistance proteins produced by these HT-29 cells in response to (i) a metabolic stress, (ii) radiations and (iii) a thermal stress applied in vitro;
stressed HCT-116 cells and stress and/or resistance proteins produced by these HCT-116 cells in response to (i) a metabolic stress, (ii) radiations and (iii) a thermal stress applied in vitro;
stressed LoVo cells and stress and/or resistance proteins produced by these LoVo cells in response to (i) a metabolic stress, (ii) radiations and (iii) a thermal stress applied in vitro;
stressed HT-29 cells and stress and/or resistance proteins produced by these HT-29 cells in response to (i) a metabolic stress, and (ii) a chemical stress applied in vitro;
stressed HCT-116 cells and stress and/or resistance proteins produced by these HCT-116 cells in response to (i) a metabolic stress, and (ii) a chemical stress applied in vitro; and
stressed LoVo cells and stress and/or resistance proteins produced by these LoVo cells in response to (i) a metabolic stress, and (ii) a chemical stress applied in vitro.

In one embodiment, the stress is radiation. Radiation is preferably at a dose sufficiently low not to kill or inactivate the cells, but sufficiently high to induce the production of stress and/or resistance proteins. In one embodiment, radiation comprises or consists of irradiating the cells with a total dose ranging from about 0.25 to about 25 Gy, preferably from about 1 to about 15 Gy, such as, e.g., with a total dose of 1, 2, 3, 4, 5, 6, 7, 8, 9, 10, 11, 12, 13, 14, or 15 Gy. In one embodiment, the irradiation period ranges from about 1 to about 20 minutes, such as, e.g., 1, 2, 3, 4, 5, 6, 7, 8, 9, 10, 11, 12, 13, 14, 15, 16, 17, 18, 19, or 20 minutes, preferably from about 1 to about 5 minutes. In one embodiment, radiation comprises or consists of irradiating the cells with a total dose of 10 Gy for a period of about 1.5 to 2 minutes, i.e., with a dose of about 5 to about 6.6 Gy/minute. In one embodiment, radiation comprises or consists of irradiating the cells with a total dose of 10 Gy for a period of about 5 minutes, i.e., with a dose of about 2 Gy/minute.

In one embodiment, the stress is a thermal stress. Thermal stress is preferably at a temperature sufficiently low not to kill or inactivate the cells, but sufficiently high to induce the production of stress and/or resistance proteins. In one embodiment, thermal stress comprises or consists of cultivating the cells at a temperature greater than 37° C., preferably ranging from about 38° C. to about 45° C., such as, e.g., at a temperature of about 38° C., 39° C., 40° C., 41° C., 42° C., 43° C., 44° C., or 45° C. In one embodiment, thermal stress is applied to the cells for a period ranging from about 15 minutes to about 4 hours, preferably from about 30 minutes to about 2 hours, such as, e.g., 30, 35, 40, 45, 50, 55, 60, 65, 70, 75, 80, 85, 90, 95, 100, 105, 110, 115, or 120 minutes. In one embodiment, thermal stress comprises or consists of cultivating the cells at a temperature of about 42° C. for a period of about 60 minutes.

In one embodiment, the stress is a chemical stress. Chemical stress is carried out by exposing the cells to at least one or several chemical agents preferably at doses sufficiently low not to kill or inactivate the cells, but sufficiently high to induce the production of stress and/or resistance proteins. In one embodiment, chemical stress comprises or consists of exposing the cells to at least one or several chemotherapeutic agents and/or alcohols. In one embodiment, chemical stress is applied to the cells for a period ranging from about 6 hours to about 120 hours, preferably from about 24 hours to about 96 hours, such as, e.g., 24, 30, 36, 42, 48, 54, 60, 66, 72, 78, 84, 90, or 96 hours. In one embodiment, chemical stress comprises or consists of exposing the cells, preferably HT-29 cells, to oxaliplatin, preferably at a concentration ranging from about 1 μM to about 20 μM, such as, e.g., 1, 2, 3, 4, 5, 6, 7, 8, 9, 10, 11, 12, 13, 14, 15, 16, 17, 18, 19, or 20 μM, more preferably at a concentration of about 13 μM, for a period of about 60 hours to about 84 hours, preferably for a period of about 72 hours. In one embodiment, chemical stress comprises or consists of exposing the cells, preferably HCT-116 cells, to SN-38 (7-ethyl-10-hydroxy-camptothecin), preferably at a concentration ranging from about 20 nM to about 400 nM, such as, e.g., 20, 40, 60, 80, 100, 120, 140, 160, 180, 200, 220, 240, 260, 280, 300, 320, 340, 360, 380, or 400 nM, more preferably at a concentration of about 31.5 nM, 100 nM or 315 nM, for a period of about 36 hours to about 60 hours, preferably for a period of about 48 hours. In one embodiment, chemical stress comprises or consists of exposing the cells, preferably LoVo cells, to fluorouracil (5-FU), preferably at a concentration ranging from about 0.5 μM to about 15 μM, such as, e.g., 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15 μM, more preferably at a concentration of about 5 μM, for a period of about 36 hours to about 60 hours, preferably for a period of about 48 hours.

In one embodiment, the stress is a metabolic stress. Metabolic stress is carried out by cultivating the cells in a non-physiological cell culture medium that does not kill or inactivate the cells, but induces the production of stress and/or resistance proteins. In one embodiment, metabolic stress comprises or consists of cultivating the cells in a depleted medium (e.g., partially or totally lacking one or several substances that are useful or even essential for the growth and/or survival of cancer cells), under hypoxia, at low pH (e.g., below pH 6.5). In one embodiment, metabolic stress comprises or consists of cultivating the cells in low serum culture conditions, such as, e.g., in a 2% FBS culture medium (instead of a 10% FBS culture medium as classically used).

In one embodiment, the composition of the invention comprises or consists of:
stressed HT-29 cells and stress and/or resistance proteins produced by these HT-29 cells in response to (i) an in vitro culture in low serum culture conditions in a 2% FBS culture medium, (ii) an in vitro radiation with a total dose of 10 Gy for a period of about 1 to about 5 minutes, and (iii) an in vitro thermic choc at a temperature of about 42° C. for a period of about 60 minutes;
stressed HCT-116 cells and stress and/or resistance proteins produced by these HCT-116 cells in response to (i) an in vitro culture in low serum culture conditions in a 2% FBS culture medium, (ii) an in vitro radiation with a total dose of 10 Gy for a period of about 1 to about 5 minutes, and (iii) an in vitro thermic choc at a temperature of about 42° C. for a period of about 60 minutes;
stressed LoVo cells and stress and/or resistance proteins produced by these LoVo cells in response to (i) an in vitro culture in low serum culture conditions in a 2% FBS culture medium, (ii) an in vitro radiation with a total dose of 10 Gy for a period of about 1 to about 5 minutes, and (iii) an in vitro thermic choc at a temperature of about 42° C. for a period of about 60 minutes;
stressed HT-29 cells and stress and/or resistance proteins produced by these HT-29 cells in response to (i) an in vitro culture in low serum culture conditions in a 2% FBS culture medium, and (ii) an in vitro exposition to about 13 μM oxaliplatin for a period of about 72 hours;
stressed HCT-116 cells and stress and/or resistance proteins produced by these HCT-116 cells in response to (i) an in vitro culture in low serum culture conditions in a 2% FBS culture medium, and (ii) an in vitro exposition to about 31.5 nM, 100 nM or 315 nM SN-38 (7-ethyl-10-hydroxy-camptothecin) for a period of about 48 hours; and
stressed LoVo cells and stress and/or resistance proteins produced by these LoVo cells in response to (i) an in vitro culture in low serum culture conditions in a 2% FBS culture medium, and (ii) an in vitro exposition to about 5 μM fluorouracil (5-FU) for a period of about 48 hours.

In one embodiment, the composition comprises HT-29, HCT-116 and LoVo cells which are non-proliferative. By “non-proliferative” or “inactive”, it is meant that these cells are not capable of cell proliferation, that is, the process by which a cell grows and divides to produce two daughter cells.

The skilled artisan is well aware of means and methods for rendering cells non-proliferative. By way of examples, cells can be rendered non-proliferative by radiation with a dose sufficiently high to kill or inactivate the cells. In one embodiment, radiation—to render the cells non-proliferative—comprises or consists of irradiating the cells with a total dose of 25 Gy or above. Alternatively or additionally, cells can be rendered non-proliferative by ethanol fixation, e.g., using from about 10% to about 50% v/v of ethanol. Alternatively or additionally, cells can be rendered non-proliferative by at least one freeze-thaw cycle. Alternatively or additionally, cells can be rendered non-proliferative by linkage to or by complexation with a means capable to confer immunogenicity, such as, e.g., by haptenation.

The skilled artisan is also familiar with means and methods to determine whether a cell is non-proliferative or not, e.g., by carrying out viability tests by cell culture (to assess the total lack of proliferation) and/or propidium iodide (which distinguishes between living cells and dead cells).

In one embodiment, non-proliferative cells are structurally intact, i.e., they display an intact plasma membrane. In one embodiment, non-proliferative cells are non-structurally intact, i.e., they do not display an intact plasma membrane. In the latter case, cells are presents in the composition in the form of membrane fragments, organelles and other cytoplasm constituents.

In one embodiment, cells have been rendered non-proliferative after having developed their resistance mechanism[s] in response to the stress[es] applied in vitro and therefore, after having produced stress and/or resistance proteins. In one embodiment, cells have been rendered non-proliferative several hours after having been stressed, preferably more than 12, 24, 36, 48, 60, 72, 84, 96 hours or more after having been stressed.

In one embodiment, the composition comprises stressed HT-29, HCT-116 and LoVo cells which overexpress at least one, at least two, at least three or the four following markers: Cmhsp70.1 (HSP70), CD227 (MUC1), CD95 (FAS receptor) and/or CD243 (MDR-1).

In one embodiment, the composition comprises stressed HT-29, HCT-116 and LoVo cells which express at least 10% more, preferably at least 15%, 20%, 25%, 30%, 35%, 40%, 45%, 50% or more of at least one, at least two, at least three or the four following markers: Cmhsp70.1 (HSP70), CD227 (MUC1), CD95 (FAS receptor) and/or CD243 (MDR-1); as compared to HT-29, HCT-116 and/or LoVo cells cultured in classical conditions such as, e.g., in 10% FBS, and not subjected to any of (i) a metabolic stress, (ii) radiations, (iii) a thermal stress and (iv) a chemical stress applied in vitro; and/or as compared to HT-29, HCT-116 and/or LoVo cells subjected to a metabolic stress, e.g., cultured in 2% FBS but not subjected to any of (i) radiations, (ii) a thermal stress and (iii) a chemical stress applied in vitro. Additionally or alternatively, at least one, at least two, at least three or the four markers Cmhsp70.1 (HSP70), CD227 (MUC1), CD95 (FAS receptor) and/or CD243 (MDR-1) are expressed in at least 10% more, preferably at least 15%, 20%, 25%, 30%, 35%, 40%, 45%, 50% or more cells in the composition than in a population of HT-29, HCT-116 and/or LoVo cells cultured in classical conditions such as, e.g., in 10% FBS, and not subjected to any of (i) a metabolic stress, (ii) radiations, (iii) a thermal stress and (iv) a chemical stress applied in vitro; and/or than in a population of HT-29, HCT-116 and/or LoVo cells subjected to a metabolic stress, e.g., cultured in 2% FBS but not subjected to any of (i) radiations, (ii) a thermal stress and (iii) a chemical stress applied in vitro. Additionally or alternatively, the mean fluorescence intensity (MFI) for at least one, at least two, at least three or the four markers Cmhsp70.1 (HSP70), CD227 (MUC1), CD95 (FAS receptor) and/or CD243 (MDR-1) is at least twice higher, preferably at least 3 times, 4 times, 5 times, 6 times or more higher in the composition than in a population of HT-29, HCT-116 and/or LoVo cells cultured in classical conditions such as, e.g., in 10% FBS, and not subjected to any of (i) a metabolic stress, (ii) radiations, (iii) a thermal stress and (iv) a chemical stress applied in vitro; and/or than in a population of HT-29, HCT-116 and/or LoVo cells subjected to a metabolic stress, e.g., cultured in 2% FBS but not subjected to any of (i) radiations, (ii) a thermal stress and (iii) a chemical stress applied in vitro.

According to the invention, the composition comprises stress and/or resistance proteins.

In one embodiment, these stress and/or resistance proteins are associated with the membrane of the HT-29, HCT-116 and LoVo cells, i.e., exposed at the surface, whether outer or inner surface, of the HT-29, HCT-116 and LoVo cells; and/or are contained within the HT-29, HCT-116 and LoVo cells, i.e., contained within their cytoplasm or any of their organelles; and/or are in a free state in the composition, i.e., not associated with the membrane of nor contained within the HT-29, HCT-116 and LoVo cells, e.g., because they were secreted by these cells.

According to the invention, the composition comprises stress and/or resistance proteins which are immunogenic. By “immunogenic” or “immunocompetent”, it is meant that the stress and/or resistance proteins are capable of eliciting an immune response (e.g., the production of antibodies) in a patient when administered to said patient.

In one embodiment, the stress and/or resistance proteins have been rendered immunogenic in the presence of a molecule or by a process capable of rendering them immunogenic. In one embodiment, the stress and/or resistance proteins have been rendered immunogenic by linkage to or by complexation with a means capable to confer immunogenicity.

Means and processes capable of conferring immunogenicity are well known to the skilled artisan. In one embodiment, the means capable of conferring immunogenicity comprise or consist of one or several molecule[s] that is/are not naturally present in the HT-29, HCT-116 and LoVo cells or in their environment. Such means are commonly referred to as “antigens”. Some antigens can trigger a humoral or cell-mediated immune response by themselves, and are then considered as “immunogens”. Some examples of such antigens include, but are not limited to, adjuvants. The skilled artisan is well aware of adjuvants suitable for use in cancer vaccines, which, when complexed with the stress and/or resistance proteins, render them immunogenic. See, e.g., Dubensky & Reed, 2010 (Semin Immunol. 22(3):155-61) or Cuzzubbo et al., 2021 (Front Immunol. 11:615240).

However, some antigens cannot initiate an immune response by themselves and require to be conjugated beforehand to a carrier, e.g., the stress and/or resistance proteins, to become immunogenic. The latter antigens are sometimes referred to as “incomplete antigens” and include, but are not limited to, haptens.

Some examples of haptens include, but are not limited to, 2,4-dinitrophenyl (DNP); 2,4-dinitrofluorobenzene; sulfanilic acid; N-iodoacetyl-N′-(5-sulfonic-naphthyl)ethylene diamine; anilin; p-amino benzoic acid; biotin; fluorescein and derivatives thereof (including FITC, TAMRA, and Texas Red); digoxigenin; 5-nitro-3-pyrazolecarbamide; 4,5-dimethoxy-2-nitrocinnamide; 2-(3,4-dimethoxyphenyl)-quinoline-4-carbamide; 2,1,3-benzoxadiazole-5-carbamide; 3-hydroxy-2-quinoxalinecarbamide, 4-(dimethylamino)azobenzene-4′-sulfonamide (DABSYL); rotenone isooxazoline; (E)-2-(2-(2-oxo-2,3-dihydro-1H-benzo[b][1,4]diazepin-4-yl)phenozy)acetamide; 7-(diethylamino)-2-oxo-2H-chromene-3-carboxylic acid; 2-acetamido-4-methyl-5-thiazolesulfonamide; and p-methoxyphenylpyrazopodophyllamide.

In one embodiment, stress and/or resistance proteins are haptenated. In one embodiment, stress and/or resistance proteins are 2,4-dinitrophenylated (DNP).

The skilled artisan is well aware of means and methods to haptenize proteins such as stress and/or resistance proteins.

Another means capable of conferring immunogenicity includes opsonization, which is a well-known process involving the binding of an opsonin to the stress and/or resistance proteins.

In one embodiment, stress and/or resistance proteins are selected from the group comprising or consisting of radiation resistance proteins, thermal stress resistance proteins, chemical stress resistance proteins, metabolic stress resistance proteins, and combination thereof.

In one embodiment, the composition specifically comprises, to detectable levels, at least one or several of the following proteins, as compared to any of HT-29, HCT-116 or LoVo cells cultured in classical conditions such as, e.g., in 10% FBS, and not subjected to any of (i) a metabolic stress, (ii) radiations (iii) a thermal stress and (iv) a chemical stress applied in vitro; and/or as compared to any of HT-29, HCT-116 or LoVo cells subjected to a metabolic stress, e.g., cultured in 2% FBS but not subjected to any of (i) radiations, (ii) a thermal stress and (iii) a chemical stress applied in vitro: tyrosine-protein kinase HCK, polypyrimidine tract-binding protein 3, E3 ubiquitin-protein ligase RNF213, serine/arginine-rich splicing factor 8, EH domain-containing protein 4, LIM domain only protein 7, DNA-directed RNA polymerase I subunit RPA2, 2′-5′-oligoadenylate synthase 3, WD repeat and HMG-box DNA-binding protein 1, beta-2-glycoprotein 1, serine/threonine-protein phosphatase PP1-gamma catalytic subunit, anillin, unconventional myosin-Ib, AP-2 complex subunit alpha-2, cyclin-dependent kinase 2, signal transducer and activator of transcription 1-alpha/beta, pumilio homolog 1, ATP-binding cassette sub-family F member 1, Rac GTPase-activating protein 1, cingulin, syntaxin-binding protein 3, mitochondrial carnitine/acylcarnitine carrier protein, importin subunit alpha-7, ribosomal protein S6 kinase alpha-4, Ras-related protein Rab-5A, ribonucleoside-diphosphate reductase large subunit, low molecular weight phosphotyrosine protein phosphatase, ribonucleoside-diphosphate reductase subunit M2, ADP-ribosylation factor-like protein 1, dynamin-2, Ras-related protein Rab-13, IST1 homolog, Forkhead box protein K1, sorbitol dehydrogenase, Bcl-2-like protein 1, tripartite motif-containing protein 29, kinesin-like protein KIF22, methylsterol monooxygenase 1, caveolae-associated protein 1, BRCA1-associated ATM activator 1, protein FAM83H, protein O-mannosyl-transferase TMTC3, inhibitor of nuclear factor kappa-B kinase-interacting protein, zinc finger CCCH-type antiviral protein 1, nucleolar protein 9, leucine zipper protein 1, polyhomeotic-like protein 2, tensin-4, LEM domain-containing protein 2, importin-4, Rho guanine nucleotide exchange factor 1, PDZ and LIM domain protein 5, UAP56-interacting factor, MMS19 nucleotide excision repair protein homolog, kinesin-like protein KIF2C, ADP-ribose glycohydrolase MACRODI, kinesin-like protein KIFC1, ATP-dependent RNA helicase DHX33, echinoderm microtubule-associated protein-like 4, fanconi anemia group I protein, EH domain-containing protein 3, opioid growth factor receptor, gamma-adducin, DNA dC-dU-editing enzyme APOBEC-3B, neuroplastin, zinc transporter 1, glutaredoxin-3, cytosolic thymidine kinase, myristoylated alanine-rich C-kinase substrate, UMP-CMP kinase, macrophage-capping protein, high mobility group protein HMGI-C, smoothelin, platelet-activating factor acetylhydrolase IB subunit gamma, transcription initiation factor TFIID subunit 9, inactive hydroxysteroid dehydrogenase-like protein 1, borealin, 1a-related protein 4, NADH dehydrogenase [ubiquinone] 1 alpha subcomplex assembly factor 2, aurora kinase B, golgi-associated plant pathogenesis-related protein 1, ATP-binding cassette sub-family F member 2, armadillo repeat-containing X-linked protein 3, ragulator complex protein LAMTOR3, probable ATP-dependent RNA helicase DDX20, V-type proton ATPase subunit H, and calcium-binding protein 39.

In one embodiment, the composition overexpresses at least one or several of the following proteins, as compared to any of HT-29, HCT-116 or LoVo cells cultured in classical conditions such as, e.g., in 10% FBS, and not subjected to any of (i) a metabolic stress, (ii) radiations (iii) a thermal stress and (iv) a chemical stress applied in vitro; and/or as compared to any of HT-29, HCT-116 or LoVo cells subjected to a metabolic stress, e.g., cultured in 2% FBS but not subjected to any of (i) radiations, (ii) a thermal stress and (iii) a chemical stress applied in vitro: HLA class I histocompatibility antigen B alpha chain, HLA class I histocompatibility antigen A alpha chain, HLA class I histocompatibility antigen C alpha chain, CD9 antigen, nuclear autoantigen Sp-100, ATP-binding cassette sub-family D member 3, ATP-binding cassette sub-family E member 1, ATP-binding cassette sub-family F member 1, ATP-binding cassette sub-family F member 2, Bcl-2-like protein 1, Bcl-2-associated transcription factor 1, cytochrome c oxidase subunit 2, mitochondrial cytochrome c oxidase subunit 4 isoform 1, mitochondrial cytochrome c oxidase subunit 5A, peroxisomal acyl-coenzyme A oxidase 1, heat shock protein beta-1, heat shock protein HSP 90-beta, heat shock cognate 71 kDa protein, heat shock 70 kDa protein 6, heat shock 70 kDa protein 4, ribonuclease inhibitor, inhibitor of nuclear factor kappa-B kinase-interacting protein, Ras-related protein Rab-5A, Ras-related protein Rab-6A, Ras-related protein Rab-5C, Ras-related protein Rab-7a, Ras-related protein Rab-13, Ras-related protein Rab-25, Ras-related protein Rab-15, Ras-related protein Rab-8A, Ras-related protein Rab-10, Ras-related protein Rap-1b, Ras-related protein Rab-1A, Ras-related protein Rap-1A, Ras-related C3 botulinum toxin substrate 1, Ras-related protein Rab-8B, Ras-related protein Rab-18, Ras-related protein Rap-2c, X-ray repair cross-complementing protein 6, DNA mismatch repair protein Msh6, MMS19 nucleotide excision repair protein homolog, protein transport protein Sec16A, sodium/potassium-transporting ATPase subunit alpha-1, facilitated glucose transporter member 1 solute carrier family 2, mitochondrial tricarboxylate transport protein, monocarboxylate transporter 1, protein transport protein Sec61 subunit beta, protein transport protein Sec61 subunit alpha isoform 1, transport and Golgi organization protein 1 homolog, adenosine 3′-phospho 5′-phosphosulfate transporter 1, transportin-1, and zinc transporter 1.

In one embodiment, the composition overexpresses at least one or several of the following membrane proteins, as compared to any of HT-29, HCT-116 or LoVo cells cultured in classical conditions such as, e.g., in 10% FBS, and not subjected to any of (i) a metabolic stress, (ii) radiations (iii) a thermal stress and (iv) a chemical stress applied in vitro; and/or as compared to any of HT-29, HCT-116 or LoVo cells subjected to a metabolic stress, e.g., cultured in 2% FBS but not subjected to any of (i) radiations, (ii) a thermal stress and (iii) a chemical stress applied in vitro: HLA class Ihistocompatibility antigen B alpha chain, HLA class I histocompatibility antigen A alpha chain, HLA class I histocompatibility antigen C alpha chain, CD9 antigen, ATP-binding cassette sub-family D member 3, Bcl-2-like protein 1, cytochrome c oxidase subunit 2, mitochondrial cytochrome c oxidase subunit 4 isoform 1, inhibitor of nuclear factor kappa-B kinase-interacting protein, sodium/potassium-transporting ATPase subunit alpha-1, facilitated glucose transporter member 1 solute carrier family 2, mitochondrial tricarboxylate transport protein, monocarboxylate transporter 1, protein transport protein Sec61 subunit beta, protein transport protein Sec61 subunit alpha isoform 1, transport and Golgi organization protein 1 homolog, adenosine 3′-phospho 5′-phosphosulfate transporter 1, and zinc transporter 1.

In one embodiment, the composition specifically comprises, to detectable levels, at least one or several of the following proteins, as compared to any of HT-29, HCT-116 or LoVo cells cultured in classical conditions such as, e.g., in 10% FBS, and not subjected to any of (i) a metabolic stress, (ii) radiations (iii) a thermal stress and (iv) a chemical stress applied in vitro; and/or as compared to any of HT-29, HCT-116 or LoVo cells subjected to a metabolic stress, e.g., cultured in 2% FBS but not subjected to any of (i) radiations, (ii) a thermal stress and (iii) a chemical stress applied in vitro: ATP-binding cassette sub-family F member 1, ATP-binding cassette sub-family F member 2, Bcl-2-like protein 1, inhibitor of nuclear factor kappa-B kinase-interacting protein, Ras-related protein Rab-5A, Ras-related protein Rab-13, MMS19 nucleotide excision repair protein homolog, and zinc transporter 1.

In one embodiment, the composition specifically comprises, to detectable levels, at least one or several of the following membrane proteins, as compared to any of HT-29, HCT-116 or LoVo cells cultured in classical conditions such as, e.g., in 10% FBS, and not subjected to any of (i) a metabolic stress, (ii) radiations (iii) a thermal stress and (iv) a chemical stress applied in vitro; and/or as compared to any of HT-29, HCT-116 or LoVo cells subjected to a metabolic stress, e.g., cultured in 2% FBS but not subjected to any of (i) radiations, (ii) a thermal stress and (iii) a chemical stress applied in vitro: Bcl-2-like protein 1, inhibitor of nuclear factor kappa-B kinase-interacting protein, and zinc transporter 1.

In one embodiment, the composition may further comprise tumor-associated antigens (TAA) and/or tumor-specific antigens (TSA).

By “tumor antigen”, it is meant an antigenic substance or molecule produced by cancer cells. Tumor antigens are classified into two categories: “tumor-specific antigens” or “TSA”, which are present only on cancer cells but not on non-cancer cells; and “tumor-associated antigens” or “TAA”, which are present on cancer and non-cancer cells.

Among common TSA are, in particular, neoantigens. As used herein, a “neoantigen” refers to an aberrant tumor-specific antigen which is encoded in cancer cells by genes comprising one or several mutations, e.g., caused by genetic instability during carcinogenesis. As a consequence, the amino acid sequence encoded by this mutated gene may itself comprise mutations leading to the production of abnormal proteins that are not found in normal cells. These mutated proteins, which may be considered as non-self protein or foreign proteins, can then be recognized by neoantigen-specific T cell receptors, activate the immune system, and lead to the immune system's attack on cancer cells. Neoantigens can also be produced by viral infection, alternative splicing and/or gene rearrangement. See, e.g., Zhang et al. (2021. Front Immunol. 12:672356) or Jiang et al. (2019. J Hematol Oncol. 12(1):93).

In one embodiment, TAA and/or TSA are specific of the HT-29, HCT-116 and LoVo cells.

In one embodiment, TAA and/or TSA are naturally immunogenic. In one embodiment, TAA and/or TSA may be rendered immunogenic, or their immunogenicity may be increased, by a molecule or a process capable of rendering them immunogenic.

Means capable to confer immunogenicity are well known to the skilled artisan and have been described above. These include, inter alia but without limitation, haptens.

In one embodiment, these TAA and/or TSA are associated with the membrane of the HT-29, HCT-116 and LoVo cells, i.e., exposed at the surface, whether outer or inner surface, of the HT-29, HCT-116 and LoVo cells; and/or are contained within the HT-29, HCT-116 and LoVo cells, i.e., contained within their cytoplasm or any of their organelles; and/or are in a free-state in the composition, i.e., not associated with the membrane of nor contained within the HT-29, HCT-116 and LoVo cells, e.g., because they were secreted by these cells.

In one embodiment, the composition is a pharmaceutical composition or a vaccine composition, and further comprises at least one pharmaceutically acceptable excipient.

Pharmaceutically acceptable excipients include, but are not limited to, water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, vegetable oils, and the like. One may additionally include suitable preservatives, stabilizers, antioxidants, antimicrobials, and buffering agents, such as, e.g., BHA, BHT, citric acid, ascorbic acid, tetracycline, and the like.

Other examples of pharmaceutically acceptable excipients that may be used in the composition of the invention include, but are not limited to, ion exchangers, alum such as aluminum phosphate or aluminium hydroxide, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In addition, some pharmaceutically acceptable excipients may include surfactants (e.g., hydroxypropylcellulose); carriers, such as, e.g., solvents and dispersion media containing, e.g., water, ethanol, polyol (e.g., glycerol, propylene glycol, and liquid polyethylene glycol, and the like), suitable mixtures thereof, and vegetable oils, such as, e.g., peanut oil and sesame oil; isotonic agents, such as, e.g., sugars or sodium chloride; coating agents, such as, e.g., lecithin; agents delaying absorption, such as, e.g., aluminum monostearate and gelatin; preservatives, such as, e.g., benzalkonium chloride, benzethonium chloride, chlorobutanol, thimerosal and the like; buffers, such as, e.g., boric acid, sodium and potassium bicarbonate, sodium and potassium borates, sodium and potassium carbonate, sodium acetate, sodium biphosphate and the like; tonicity agents, such as, e.g., dextran 40, dextran 70, dextrose, glycerin, potassium chloride, propylene glycol, sodium chloride; antioxidants and stabilizers, such as, e.g., sodium bisulfite, sodium metabisulfite, sodium thiosulfite, thiourea and the like; nonionic wetting or clarifying agents, such as, e.g., polysorbate 80, polysorbate 20, poloxamer 282 and tyloxapol; viscosity modifying agents, such as, e.g., dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose; and the like.

Examples of adjuvants include, but are not limited to, helper peptide, aluminum salts (such as, e.g., aluminum hydroxide (also named alum), aluminum phosphate, and the like), Freund's incomplete adjuvant, Freund's complete adjuvant, saponin, Merck adjuvant 65, Smith-Kline Beecham adjuvant AS-2. Aquilla adjuvant QS-21, MPL™ immunostimulant, 3d-MPL, LEIF, calcium salts, iron salts, zinc salts, acylated tyrosine, acylated sugars, cationically-derivatized polysaccharides, anionically-derivatized polysaccharides, polyphosphazenes, biodegradable microspheres, monophosphoryl lipid A, muramyl tripeptide phosphatidyl ethanolamine, cytokines (such as, e.g., interleukin-2, interleukin-12, interleukin-4, interleukin-7 and the like), CpG-containing oligonucleotide, and combinations thereof.

In one embodiment, the composition comprises from about 10⁵ to about 10⁸ stressed HT-29, HCT-116 and LoVo cells, preferably from about 10⁶ to about 10⁷ stressed HT-29, HCT-116 and LoVo cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed HT-29, HCT-116 and LoVo cells. In one embodiment, the composition comprises about 3×10⁶ stressed HT-29, HCT-116 and LoVo cells.

In one embodiment, the composition comprises from about 10⁶ to about 10⁹ stressed HT-29, HCT-116 and LoVo cells per mL of composition, preferably from about 10⁷ to about 10⁸ stressed HT-29, HCT-116 and LoVo cells per mL of composition, such as, about 1×10⁷, 2×10⁷, 3×10⁷, 4×10⁷, 5×10⁷, 6×10⁷, 7×10⁷, 8×10⁷, 9×10⁷, or 1×10⁸ stressed HT-29, HCT-116 and LoVo cells per mL of composition. In one embodiment, the composition comprises about 3×10⁷ stressed HT-29, HCT-116 and LoVo cells per mL of composition.

In one embodiment, the composition comprises an equal ratio of stressed HT-29, HCT-116 and LoVo cells (i.e., about 1:1:1). In one embodiment, the composition comprises at least 1.5, 2 or 2.5 times more stressed HT-29 than stressed HCT-116 or LoVo cells. In one embodiment, the composition comprises at least 1.5, 2 or 2.5 times more stressed HCT-116 than stressed HT-29 or LoVo cells. In one embodiment, the composition comprises at least 1.5, 2 or 2.5 times more stressed LoVo than stressed HT-29 or HCT-116 cells.

In one embodiment, the composition comprises:
from about 10⁵ to about 10⁸ stressed HT-29 cells, preferably from about 10⁶ to about 10⁷ stressed HT-29 cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed HT-29 cells,
from about 10⁵ to about 10⁸ stressed HCT-116 cells, preferably from about 10⁶ to about 10⁷ stressed HCT-116 cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed HCT-116 cells, and
from about 10⁵ to about 10⁸ stressed LoVo cells, preferably from about 10⁶ to about 10⁷ stressed LoVo cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed LoVo cells.

### Intermediate Products

The present invention also relates to intermediate compositions useful in the preparation of the composition described above.

In one embodiment, the intermediate compositions comprise at least one pharmaceutically acceptable excipient, as defined above.

In one embodiment, the intermediate composition comprises or consists of (i) stressed HT-29, HCT-116 or LoVo cells, and (ii) stress and/or resistance proteins, wherein the stress and/or resistance proteins were produced by these HT-29, HCT-116 or LoVo cells in response to (i) a metabolic stress, (ii) radiations and (iii) a thermal stress applied in vitro. Radiations, thermal stresses and metabolic stresses have been described above.

In one embodiment, the stress and/or resistance proteins are immunogenic. Means and methods for rendering stress and/or resistance proteins immunogenic have been described above.

In one embodiment, the intermediate composition may further comprise tumor-associated antigens (TAA) and/or tumor-specific antigens (TSA), as described above. In one embodiment, TAA and/or TSA are specific of the HT-29, HCT-116 or LoVo cells. In one embodiment, TAA and/or TSA are naturally immunogenic. In one embodiment, TAA and/or TSA may be rendered immunogenic, or their immunogenicity may be increased, by linkage to or by complexation with a means capable to confer immunogenicity. Means capable to confer immunogenicity are well known to the skilled artisan and have been described above. These include, inter alia but without limitation, haptens.

In one embodiment, the intermediate composition comprises or consists of (i) stressed HT-29 cells and (ii) stress and/or resistance proteins produced by these HT-29 cells in response to:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro radiation with a total dose ranging from about 0.25 to about 25 Gy, preferably from about 1 to about 15 Gy, such as, e.g., with a total dose of 1, 2, 3, 4, 5, 6, 7, 8, 9, 10, 11, 12, 13, 14, or 15 Gy. In one embodiment, the irradiation period ranges from about 1 to about 20 minutes, such as, e.g., 1, 2, 3, 4, 5, 6, 7, 8, 9, 10, 11, 12, 13, 14, 15, 16, 17, 18, 19, or 20 minutes, preferably from about 1 to about 5 minutes, and
(iii) an in vitro thermic choc at a temperature ranging from about 38° C. to about 45° C., such as, e.g., at a temperature of about 38° C., 39° C., 40° C., 41° C., 42° C., 43° C., 44° C., or 45° C., applied to the cells for a period ranging from about 15 minutes to about 4 hours, preferably from about 30 minutes to about 2 hours, such as, e.g., 30, 35, 40, 45, 50, 55, 60, 65, 70, 75, 80, 85, 90, 95, 100, 105, 110, 115, or 120 minutes.

In one embodiment, the intermediate composition comprises or consists of (i) stressed HT-29 cells and (ii) stress and/or resistance proteins produced by these HT-29 cells in response to
(i) an in vitro culture in low serum culture conditions in a 2% FBS culture medium,
(ii) an in vitro radiation with a total dose of 10 Gy for a period of about 1 to about 5 minutes, and
(iii) an in vitro thermic choc at a temperature of about 42° C. for a period of about 60 minutes.

This intermediate composition is herein referred to as “HT-29 DS-A”.

In one embodiment, HT-29 DS-A comprises stress and/or resistance proteins selected from the group comprising or consisting of radiation resistance proteins, thermal stress resistance proteins, metabolic stress resistance proteins, and combination thereof.

In one embodiment, HT-29 DS-A cells overexpress at least one, at least two or the three following markers: Cmhsp70.1 (HSP70), CD227 (MUC1) and/or CD107 (LAMP-1).

In one embodiment, HT-29 DS-A cells express at least 10% more, preferably at least 15%, 20%, 25%, 30%, 35%, 40%, 45%, 50% or more of at least one, at least two or the three following markers: Cmhsp70.1 (HSP70), CD227 (MUC1) and/or CD107 (LAMP-1); as compared to HT-29 cells cultured in classical conditions such as, e.g., in 10% FBS, and not subjected to any of (i) a metabolic stress, (ii) radiations and (iii) a thermal stress applied in vitro; and/or as compared to HT-29 cells subjected to a metabolic stress, e.g., cultured in 2% FBS but not subjected to any of (i) radiations and (ii) a thermal stress applied in vitro. Additionally or alternatively, at least one, at least two or the three markers Cmhsp70.1 (HSP70), CD227 (MUC1) and CD107 (LAMP-1) are expressed in at least 10% more, preferably at least 15%, 20%, 25%, 30%, 35%, 40%, 45%, 50% or more cells in a population of HT-29 DS-A cells than in a population of HT-29 cells cultured in classical conditions such as, e.g., in 10% FBS, and not subjected to any of (i) a metabolic stress, (ii) radiations and (iii) a thermal stress applied in vitro; and/or than in a population of HT-29 cells subjected to a metabolic stress, e.g., cultured in 2% FBS but not subjected to any of (i) radiations and (ii) a thermal stress applied in vitro.

Additionally or alternatively, the mean fluorescence intensity (MFI) for at least one, at least two or the three markers Cmhsp70.1 (HSP70), CD227 (MUC1) and/or CD107 (LAMP-1) is at least twice higher, preferably at least 3 times, 4 times, 5 times, 6 times or more higher in a population of HT-29 DS-A cells than in a population of HT-29 cells cultured in classical conditions such as, e.g., in 10% FBS, and not subjected to any of (i) a metabolic stress, (ii) radiations and (iii) a thermal stress applied in vitro; and/or than in a population of HT-29 cells subjected to a metabolic stress, e.g., cultured in 2% FBS but not subjected to any of (i) radiations and (ii) a thermal stress applied in vitro.

In one embodiment, HT-29 DS-A cells overexpress at least one or several of the following proteins, as compared to HT-29 cells subjected to a metabolic stress, e.g., cultured in 2% FBS but not subjected to any of (i) radiations and (ii) a thermal stress applied in vitro: poly(rC)-binding protein 2, rho guanine nucleotide exchange factor 1, golgin subfamily B member 1, beta-2-glycoprotein 1, plakophilin-3, protein FAM83H, protein transport protein Sec16A, inverted formin-2, anillin, protein ECT2, plectin, epiplakin, proliferation marker protein Ki-67, vesicular integral-membrane protein VIP36, and lysosome-associated membrane glycoprotein 1.

In one embodiment, HT-29 DS-A cells specifically express, to detectable levels, at least one or several of the following proteins, as compared to HT-29 cells subjected to a metabolic stress, e.g., cultured in 2% FBS but not subjected to any of (i) radiations and (ii) a thermal stress applied in vitro: poly(rC)-binding protein 2, rho guanine nucleotide exchange factor 1, golgin subfamily B member 1, beta-2-glycoprotein 1, plakophilin-3, protein FAM83H, protein transport protein Sec16A, inverted formin-2, anillin, and protein ECT2.

In one embodiment, HT-29 DS-A cells overexpress at least one or several of the following membrane and/or cell surface proteins, as compared to HT-29 cells subjected to a metabolic stress, e.g., cultured in 2% FBS but not subjected to any of (i) radiations and (ii) a thermal stress applied in vitro: vesicular integral-membrane protein VIP36, lysosome-associated membrane glycoprotein 1, ribosome-binding protein 1, Kunitz-type protease inhibitor 2, lysophospholipid acyltransferase 5, emerin, protein LYRIC, elongation of very long chain fatty acids protein 1, and protein transport protein Sec61 subunit gamma.

In one embodiment, HT-29 DS-A cells overexpress at least one or several of the following cell surface proteins, as compared to HT-29 cells subjected to a metabolic stress, e.g., cultured in 2% FBS but not subjected to any of (i) radiations and (ii) a thermal stress applied in vitro: heat shock-related 70 kDa protein, annexin, anoctamin-6, immunoglobulin superfamily member 3, serotransferrin, tumor necrosis factor receptor superfamily member 10B, clusterin, furin, HLA class II histocompatibility antigen gamma chain, CD109 antigen, chloride intracellular channel protein 4, protocadherin fat 1, Natural resistance-associated macrophage protein 2, tumor necrosis factor receptor superfamily member 10A, calpain-5, MHC class I polypeptide-related sequence A, high mobility group protein B1, tetraspanin-15, UL16-binding protein 2, integrin beta-7, sonic hedgehog protein, toll-like receptor 3, beta-2-glycoprotein 1, tissue factor, proprotein convertase subtilisin/kexin type 6, endothelial protein C receptor, volume-regulated anion channel subunit LRRC8A, cadherin EGF LAG seven-pass G-type receptor 3, zinc transporter ZIP6, HLA class II histocompatibility antigen DM alpha chain, cystine/glutamate transporter, lysophosphatidic acid receptor 2, syndecan-1, hyaluronidase-2, integrin alpha-4, histidine-rich glycoprotein, transforming growth factor beta-1 proprotein, and metalloproteinase inhibitor 2.

In one embodiment, the intermediate composition comprises or consists of (i) stressed HCT-116 cells and (ii) stress and/or resistance proteins produced by these HCT-116 cells in response to:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro radiation with a total dose ranging from about 0.25 to about 25 Gy, preferably from about 1 to about 15 Gy, such as, e.g., with a total dose of 1, 2, 3, 4, 5, 6, 7, 8, 9, 10, 11, 12, 13, 14, or 15 Gy. In one embodiment, the irradiation period ranges from about 1 to about 20 minutes, such as, e.g., 1, 2, 3, 4, 5, 6, 7, 8, 9, 10, 11, 12, 13, 14, 15, 16, 17, 18, 19, or 20 minutes, preferably from about 1 to about 5 minutes, and
(iii) an in vitro thermic choc at a temperature ranging from about 38° C. to about 45° C., such as, e.g., at a temperature of about 38° C., 39° C., 40° C., 41° C., 42° C., 43° C., 44° C., or 45° C., applied to the cells for a period ranging from about 15 minutes to about 4 hours, preferably from about 30 minutes to about 2 hours, such as, e.g., 30, 35, 40, 45, 50, 55, 60, 65, 70, 75, 80, 85, 90, 95, 100, 105, 110, 115, or 120 minutes.

In one embodiment, the intermediate composition comprises or consists of (i) stressed HCT-116 cells and (ii) stress and/or resistance proteins produced by these HCT-116 cells in response to
(i) an in vitro culture in low serum culture conditions in a 2% FBS culture medium,
(ii) an in vitro radiation with a total dose of 10 Gy for a period of about 1 to about 5 minutes, and
(iii) an in vitro thermic choc at a temperature of about 42° C. for a period of about 60 minutes.

This intermediate composition is herein referred to as “HCT-116 DS-A”.

In one embodiment, HCT-116 DS-A comprises stress and/or resistance proteins selected from the group comprising or consisting of radiation resistance proteins, thermal stress resistance proteins, metabolic stress resistance proteins, and combination thereof.

In one embodiment, HCT-116 DS-A cells overexpress at least one or the two following markers: Cmhsp70.1 (HSP70) and/or CD227 (MUC1).

In one embodiment, HCT-116 DS-A cells express at least 10% more, preferably at least 15%, 20%, 25%, 30%, 35%, 40%, 45%, 50% or more of at least one or the two following markers: Cmhsp70.1 (HSP70) and/or CD227 (MUC1); as compared to HCT-116 cells cultured in classical conditions such as, e.g., in 10% FBS, and not subjected to any of (i) a metabolic stress, (ii) radiations and (iii) a thermal stress applied in vitro; and/or as compared to HCT-116 cells subjected to a metabolic stress, e.g., cultured in 2% FBS but not subjected to any of (i) radiations and (ii) a thermal stress applied in vitro. Additionally or alternatively, at least one or the two markers Cmhsp70.1 (HSP70) and CD227 (MUC1) are expressed in at least 10% more, preferably at least 15%, 20%, 25%, 30%, 35%, 40%, 45%, 50% or more cells in a population of HCT-116 DS-A cells than in a population of HCT-116 cells cultured in classical conditions such as, e.g., in 10% FBS, and not subjected to any of (i) a metabolic stress, (ii) radiations and (iii) a thermal stress applied in vitro; and/or than in a population of HCT-116 cells subjected to a metabolic stress, e.g., cultured in 2% FBS but not subjected to any of (i) radiations and (ii) a thermal stress applied in vitro. Additionally or alternatively, the mean fluorescence intensity (MFI) for at least one or the two markers Cmhsp70.1 (HSP70) and CD227 (MUC1) is at least twice higher, preferably at least 3 times, 4 times, 5 times, 6 times or more higher in a population of HCT-116 DS-A cells than in a population of HCT-116 cells cultured in classical conditions such as, e.g., in 10% FBS, and not subjected to any of (i) a metabolic stress, (ii) radiations and (iii) a thermal stress applied in vitro; and/or than in a population of HCT-116 cells subjected to a metabolic stress, e.g., cultured in 2% FBS but not subjected to any of (i) radiations and (ii) a thermal stress applied in vitro.

In one embodiment, HCT-116 DS-A cells overexpress at least one or several of the following proteins, as compared to HCT-116 cells subjected to a metabolic stress, e.g., cultured in 2% FBS but not subjected to any of (i) radiations and (ii) a thermal stress applied in vitro: poly(rC)-binding protein 2, rho guanine nucleotide exchange factor 1, golgin subfamily B member 1, beta-2-glycoprotein 1, plakophilin-3, protein FAM83H, protein transport protein Sec16A, inverted formin-2, anillin, protein ECT2, plectin, epiplakin, proliferation marker protein Ki-67, vesicular integral-membrane protein VIP36, and lysosome-associated membrane glycoprotein 1.

In one embodiment, HCT-116 DS-A cells specifically express, to detectable levels, at least one or several of the following proteins, as compared to HCT-116 cells subjected to a metabolic stress, e.g., cultured in 2% FBS but not subjected to any of (i) radiations and (ii) a thermal stress applied in vitro: poly(rC)-binding protein 2, rho guanine nucleotide exchange factor 1, golgin subfamily B member 1, beta-2-glycoprotein 1, plakophilin-3, protein FAM83H, protein transport protein Sec16A, inverted formin-2, anillin, and protein ECT2.

In one embodiment, HCT-116 DS-A cells overexpress at least one or several of the following membrane and/or cell surface proteins, as compared to HCT-116 cells subjected to a metabolic stress, e.g., cultured in 2% FBS but not subjected to any of (i) radiations and (ii) a thermal stress applied in vitro: integrin alpha-6, lysosome-associated membrane glycoprotein 1, ribosome-binding protein 1, delta(14)-sterol reductase LBR, mitochondrial proton/calcium exchanger protein, RRP12-like protein, extended syptotagmin-1, protein transport protein Sec61 subunit alpha isoform 1, solute carrier family 2, facilitated glucose transporter member 1, desmoglein-2, kinectin, protein LYRIC, sphingosine-1-phosphate lyase 1, vesicle-associated membrane protein-associated protein A, lysophospholipid acyltransferase 7, Sigl recognition particle receptor subunit beta, torsin-1A-interacting protein 1, ER membrane protein complex subunit 1, decarboxylating sterol-4-alpha-carboxylate 3-dehydrogese, membrane-associated progesterone receptor component 1, CD44 antigen, MICOS complex subunit MIC26, transmembrane emp24 domain-containing protein 4, ORM1-like protein 2, dolichol-phosphate mannosyltransferase subunit 3, cytoskeleton-associated protein 4, transmembrane protein 43, retinol dehydrogese 11, Sigl peptidase complex subunit 2, serine palmitoyltransferase 2, coiled-coil domain-containing protein 47, very-long-chain enoyl-CoA reductase, protocadherin Fat 1, transmembrane emp24 domain-containing protein 7, caveolin-1, NF-X1-type zinc finger protein NFXL1, receptor expression-enhancing protein 6, mitochondrial HIG1 domain family member 2A, small integral membrane protein 20, DH dehydrogese [ubiquinone] 1 alpha subcomplex subunit 13, DH dehydrogese [ubiquinone] 1 beta subcomplex subunit 3, mitochondrial thiamine pyrophosphate carrier, mitochondrial fission 1 protein, amine oxidase [flavin-containing] B, mitochondrial inner membrane protein OXA1L, 2-hydroxyacyl-CoA lyase 2, nicalin, plasma membrane calcium-transporting ATPase 1, myoferlin, and cation-independent mannose-6-phosphate receptor.

In one embodiment, the intermediate composition comprises or consists of (i) stressed LoVo cells and (ii) stress and/or resistance proteins produced by these LoVo cells in response to:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro radiation with a total dose ranging from about 0.25 to about 25 Gy, preferably from about 1 to about 15 Gy, such as, e.g., with a total dose of 1, 2, 3, 4, 5, 6, 7, 8, 9, 10, 11, 12, 13, 14, or 15 Gy. In one embodiment, the irradiation period ranges from about 1 to about 20 minutes, such as, e.g., 1, 2, 3, 4, 5, 6, 7, 8, 9, 10, 11, 12, 13, 14, 15, 16, 17, 18, 19, or 20 minutes, preferably from about 1 to about 5 minutes, and
(iii) an in vitro thermic choc at a temperature ranging from about 38° C. to about 45° C., such as, e.g., at a temperature of about 38° C., 39° C., 40° C., 41° C., 42° C., 43° C., 44° C., or 45° C., applied to the cells for a period ranging from about 15 minutes to about 4 hours, preferably from about 30 minutes to about 2 hours, such as, e.g., 30, 35, 40, 45, 50, 55, 60, 65, 70, 75, 80, 85, 90, 95, 100, 105, 110, 115, or 120 minutes.

In one embodiment, the intermediate composition comprises or consists of (i) stressed LoVo cells and (ii) stress and/or resistance proteins produced by these LoVo cells in response to
(i) an in vitro culture in low serum culture conditions in a 2% FBS culture medium,
(ii) an in vitro radiation with a total dose of 10 Gy for a period of about 1 to about 5 minutes, and
(iii) an in vitro thermic choc at a temperature of about 42° C. for a period of about 60 minutes.

This intermediate composition is herein referred to as “LoVo DS-A”.

In one embodiment, LoVo DS-A comprises stress and/or resistance proteins selected from the group comprising or consisting of radiation resistance proteins, thermal stress resistance proteins, metabolic stress resistance proteins, and combination thereof.

In one embodiment, LoVo DS-A cells overexpress at least one, at least two or the three following markers: Cmhsp70.1 (HSP70), CD227 (MUC1) and/or CD107 (LAMP-1).

In one embodiment, LoVo DS-A cells express at least 10% more, preferably at least 15%, 20%, 25%, 30%, 35%, 40%, 45%, 50% or more of at least one, at least two or the three following markers: Cmhsp70.1 (HSP70), CD227 (MUC1) and/or CD107 (LAMP-1); as compared to LoVo cells cultured in classical conditions such as, e.g., in 10% FBS, and not subjected to any of (i) a metabolic stress, (ii) radiations and (iii) a thermal stress applied in vitro; and/or as compared to LoVo cells subjected to a metabolic stress, e.g., cultured in 2% FBS but not subjected to any of (i) radiations and (ii) a thermal stress applied in vitro. Additionally or alternatively, at least one, at least two or the three markers Cmhsp70.1 (HSP70), CD227 (MUC1) and CD107 (LAMP-1) are expressed in at least 10% more, preferably at least 15%, 20%, 25%, 30%, 35%, 40%, 45%, 50% or more cells in a population of LoVo DS-A cells than in a population of LoVo cells cultured in classical conditions such as, e.g., in 10% FBS, and not subjected to any of (i) a metabolic stress, (ii) radiations and (iii) a thermal stress applied in vitro; and/or than in a population of LoVo cells subjected to a metabolic stress, e.g., cultured in 2% FBS but not subjected to any of (i) radiations and (ii) a thermal stress applied in vitro. Additionally or alternatively, the mean fluorescence intensity (MFI) for at least one, at least two or the three markers Cmhsp70.1 (HSP70), CD227 (MUC1) and CD107 (LAMP-1) is at least twice higher, preferably at least 3 times, 4 times, 5 times, 6 times or more higher in a population of LoVo DS-A cells than in a population of LoVo cells cultured in classical conditions such as, e.g., in 10% FBS, and not subjected to any of (i) a metabolic stress, (ii) radiations and (iii) a thermal stress applied in vitro; and/or than in a population of LoVo cells subjected to a metabolic stress, e.g., cultured in 2% FBS but not subjected to any of (i) radiations and (ii) a thermal stress applied in vitro.

In one embodiment, LoVo DS-A cells overexpress at least one or several of the following proteins, as compared to LoVo cells subjected to a metabolic stress, e.g., cultured in 2% FBS but not subjected to any of (i) radiations and (ii) a thermal stress applied in vitro: poly(rC)-binding protein 2, rho guanine nucleotide exchange factor 1, golgin subfamily B member 1, beta-2-glycoprotein 1, plakophilin-3, protein FAM83H, protein transport protein Sec16A, inverted formin-2, anillin, protein ECT2, plectin, epiplakin, proliferation marker protein Ki-67, vesicular integral-membrane protein VIP36, and lysosome-associated membrane glycoprotein 1.

In one embodiment, LoVo DS-A cells specifically express, to detectable levels, at least one or several of the following proteins, as compared to LoVo cells subjected to a metabolic stress, e.g., cultured in 2% FBS but not subjected to any of (i) radiations and (ii) a thermal stress applied in vitro: poly(rC)-binding protein 2, rho guanine nucleotide exchange factor 1, golgin subfamily B member 1, beta-2-glycoprotein 1, plakophilin-3, protein FAM83H, protein transport protein Sec16A, inverted formin-2, anillin, and protein ECT2.

In one embodiment, LoVo DS-A cells overexpress at least one or several of the following membrane and/or cell surface proteins, as compared to LoVo cells subjected to a metabolic stress, e.g., cultured in 2% FBS but not subjected to any of (i) radiations and (ii) a thermal stress applied in vitro: vesicular integral-membrane protein VIP36, nucleolar complex protein 4 homolog, Sigl peptidase complex catalytic subunit SEC11A, Sigl recognition particle receptor subunit beta, microsomal glutathione S-transferase 1, DPH-cytochrome P450 reductase, very-long-chain (3R)-3-hydroxyacyl-CoA dehydratase 3, mannosyl-oligosaccharide glucosidase, oxysterol-binding protein-related protein 8, receptor expression-enhancing protein 5, MICOS complex subunit MIC26, beta-1,3-galactosyl-O-glycosyl-glycoprotein beta-1,6-N-acetylglucosaminyltransferase 3, mitochondrial ATP synthase membrane subunit DAPIT, mitochondrial ATP synthase subunit f, CDGSH iron-sulfur domain-containing protein 2, vitamin K epoxide reductase complex subunit 1-like protein 1, protein FAM162A, erlin-1, long-chain-fatty-acid—CoA ligase 1, CYB5B_HUMAN Cytochrome b5 type B, retinol dehydrogese 11, transmembrane emp24 domain-containing protein 2, very-long-chain enoyl-CoA reductase, 3-beta-hydroxysteroid-Delta(8), Delta(7)-isomerase, monocarboxylate transporter 1, HLA class I histocompatibility antigen B alpha chain, erlin-2, CD9 antigen, ATP synthase protein 8, mitochondrial fission process protein 1, cytochrome c oxidase subunit NDUFA4, DH dehydrogese [ubiquinone] 1 alpha subcomplex subunit 13, DH dehydrogese [ubiquinone] 1 beta subcomplex subunit 4, DH dehydrogese [ubiquinone] 1 beta subcomplex subunit 3, mitochondrial fission 1 protein, and BRI3-binding protein.

In one embodiment, the intermediate composition comprises or consists of (i) stressed HT-29, HCT-116 or LoVo cells, and (ii) stress and/or resistance proteins, wherein the stress and/or resistance proteins were produced by these HT-29, HCT-116 or LoVo cells in response to (i) a metabolic stress, and (ii) a chemical stress applied in vitro. Chemical stresses and metabolic stresses have been described above.

In one embodiment, the stress and/or resistance proteins are immunogenic. Means and methods for rendering stress and/or resistance proteins immunogenic have been described above.

In one embodiment, the intermediate composition may further comprise tumor-associated antigens (TAA) and/or tumor-specific antigens (TSA), as described above. In one embodiment, TAA and/or TSA are specific of the HT-29, HCT-116 or LoVo cells. In one embodiment, TAA and/or TSA are naturally immunogenic. In one embodiment, TAA and/or TSA may be rendered immunogenic, or their immunogenicity may be increased, by linkage to or by complexation with a means capable to confer immunogenicity. Means capable to confer immunogenicity are well known to the skilled artisan and have been described above. These include, inter alia but without limitation, haptens.

In one embodiment, the intermediate composition comprises or consists of (i) stressed HT-29 cells and (ii) stress and/or resistance proteins produced by these HT-29 cells in response to
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro exposition to at least one or several chemotherapeutic agents and/or alcohols, for a period ranging from about 6 hours to about 120 hours, preferably from about 24 hours to about 96 hours, such as, e.g., 24, 30, 36, 42, 48, 54, 60, 66, 72, 78, 84, 90, or 96 hours.

In one embodiment, the intermediate composition comprises or consists of (i) stressed HT-29 cells and (ii) stress and/or resistance proteins produced by these HT-29 cells in response to
(i) an in vitro culture in low serum culture conditions in a 2% FBS culture medium, and
(ii) an in vitro exposition to about 13 μM oxaliplatin for a period of about 72 hours.

This intermediate composition is herein referred to as “HT-29 DS-B”.

In one embodiment, HT-29 DS-B comprises stress and/or resistance proteins selected from the group comprising or consisting of radiation resistance proteins, thermal stress resistance proteins, metabolic stress resistance proteins, and combination thereof.

In one embodiment, HT-29 DS-B cells overexpress at least one, at least two or the three following markers: CD54 (ICAM-1), CD95 (FAS receptor) and/or CD107 (LAMP-1).

In one embodiment, HT-29 DS-B cells express at least 10% more, preferably at least 15%, 20%, 25%, 30%, 35%, 40%, 45%, 50% or more of at least one, at least two or the three following markers: CD54 (ICAM-1), CD95 (FAS receptor) and/or CD107 (LAMP-1); as compared to HT-29 cells cultured in classical conditions such as, e.g., in 10% FBS, and not subjected to any of (i) a metabolic stress, and (ii) a chemical stress applied in vitro (e.g., an in vitro exposition to about 13 μM oxaliplatin for a period of about 72 hours); and/or as compared to HT-29 cells subjected to a metabolic stress, e.g., cultured in 2% FBS but not subjected to a chemical stress applied in vitro (e.g., an in vitro exposition to about 13 μM oxaliplatin for a period of about 72 hours). Additionally or alternatively, at least one, at least two or the three markers CD54 (ICAM-1), CD95 (FAS receptor) and/or CD107 (LAMP-1) are expressed in at least 10% more, preferably at least 15%, 20%, 25%, 30%, 35%, 40%, 45%, 50% or more cells in a population of HT-29 DS-B cells than in a population of HT-29 cells cultured in classical conditions such as, e.g., in 10% FBS, and not subjected to any of (i) a metabolic stress, and (ii) a chemical stress applied in vitro (e.g., an in vitro exposition to about 13 μM oxaliplatin for a period of about 72 hours); and/or than in a population of HT-29 cells subjected to a metabolic stress, e.g., cultured in 2% FBS but not subjected to a chemical stress applied in vitro (e.g., an in vitro exposition to about 13 μM oxaliplatin for a period of about 72 hours). Additionally or alternatively, the mean fluorescence intensity (MFI) for at least one, at least two or the three markers CD54 (TCAM-1), CD95 (FAS receptor) and/or CD107 (LAMP-1) is at least twice higher, preferably at least 3 times, 4 times, 5 times, 6 times or more higher in a population of HT-29 DS-B cells than in a population of HT-29 cells cultured in classical conditions such as, e.g., in 10% FBS, and not subjected to any of (i) a metabolic stress, and (ii) a chemical stress applied in vitro (e.g., an in vitro exposition to about 13 μM oxaliplatin for a period of about 72 hours); and/or than in a population of HT-29 cells subjected to a metabolic stress, e.g., cultured in 2% FBS but not subjected to a chemical stress applied in vitro (e.g., an in vitro exposition to about 13 μM oxaliplatin for a period of about 72 hours).

In one embodiment, HT-29 DS-B cells overexpress at least one or several of the following proteins, as compared to HT-29 cells subjected to a metabolic stress, e.g., cultured in 2% FBS but not subjected to a chemical stress applied in vitro: beta-2-glycoprotein 1, histone H2A type 1, poly(rC)-binding protein 2, kinectin, plectin, and NADH dehydrogenase [ubiquinone] 1 alpha subcomplex subunit 7.

In one embodiment, HT-29 DS-B cells specifically express, to detectable levels, at least one or several of the following proteins, as compared to HT-29 cells subjected to a metabolic stress, e.g., cultured in 2% FBS but not subjected to a chemical stress applied in vitro: beta-2-glycoprotein 1, histone H2A type 1, and poly(rC)-binding protein 2.

In one embodiment, HT-29 DS-B cells overexpress at least one or several of the following membrane and/or cell surface proteins, as compared to HT-29 cells subjected to a metabolic stress, e.g., cultured in 2% FBS but not subjected to a chemical stress applied in vitro: ribosome-binding protein 1, and NADPH-cytochrome P450 reductase.

In one embodiment, HT-29 DS-B cells overexpress at least one or several of the following cell surface proteins, as compared to HT-29 cells subjected to a metabolic stress, e.g., cultured in 2% FBS but not subjected to a chemical stress applied in vitro: CD109 antigen, HLA class II histocompatibility antigen gamma chain, HLA class I histocompatibility antigen alpha chain F, hyaluronan mediated motility receptor, integrin beta-8, integrin beta-3, proprotein convertase subtilisin/kexin type 6, clusterin, serotransferrin, natural resistance-associated macrophage protein 2, MHC class I polypeptide-related sequence A, tumor necrosis factor receptor superfamily member 10B, endothelial protein C receptor, cadherin EGF LAG seven-pass G-type receptor 3, tumor necrosis factor receptor superfamily member 10A, cystine/glutamate transporter, tissue factor, transforming growth factor beta-1 proprotein, immunoglobulin superfamily member 3, anoctamin-6, metalloproteinase inhibitor 2, toll-like receptor 3, volume-regulated anion channel subunit LRRC8A, tetraspanin-15, zinc transporter ZIP6, furin, protocadherin fat 1, hyaluronidase-2, lysophosphatidic acid receptor 2, high mobility group protein B1, chloride intracellular channel protein 4, UL16-binding protein 2, calpain-5, annexin A9, histidine-rich glycoprotein, integrin alpha-4, and heat shock-related 70 kDa protein 2.

In one embodiment, the intermediate composition comprises or consists of (i) stressed HCT-116 cells and (ii) stress and/or resistance proteins produced by these HCT-116 cells in response to
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro exposition to at least one or several chemotherapeutic agents and/or alcohols, for a period ranging from about 6 hours to about 120 hours, preferably from about 24 hours to about 96 hours, such as, e.g., 24, 30, 36, 42, 48, 54, 60, 66, 72, 78, 84, 90, or 96 hours.

In one embodiment, the intermediate composition comprises or consists of (i) stressed HCT-116 cells and (ii) stress and/or resistance proteins produced by these HCT-116 cells in response to
(i) an in vitro culture in low serum culture conditions in a 2% FBS culture medium, and
(ii) an in vitro exposition to about 31.5 nM, 100 nM or 315 nM SN-38 (7-ethyl-10-hydroxy-camptothecin) for a period of about 48 hours.

This intermediate composition is herein referred to as “HCT-116 DS-B”.

In one embodiment, HCT-116 DS-B comprises stress and/or resistance proteins selected from the group comprising or consisting of radiation resistance proteins, thermal stress resistance proteins, metabolic stress resistance proteins, and combination thereof.

In one embodiment, HCT-116 DS-B cells overexpress the following marker: CD66 (CEA).

In one embodiment, HCT-116 DS-B cells express at least 10% more, preferably at least 15%, 20%, 25%, 30%, 35%, 40%, 45%, 50% or more of the marker CD66 (CEA) as compared to HCT-116 cells cultured in classical conditions such as, e.g., in 10% FBS, and not subjected to any of (i) a metabolic stress, and (ii) a chemical stress applied in vitro (e.g., an in vitro exposition to about 31.5 nM, 100 nM or 315 nM SN-38 (7-ethyl-10-hydroxy-camptothecin) for a period of about 48 hours); and/or as compared to HCT-116 cells subjected to a metabolic stress, e.g., cultured in 2% FBS but not subjected to a chemical stress applied in vitro (e.g., an in vitro exposition to about 31.5 nM, 100 nM or 315 nM SN-38 (7-ethyl-10-hydroxy-camptothecin) for a period of about 48 hours). Additionally or alternatively, the marker CD66 (CEA) is expressed in at least 10% more, preferably at least 15%, 20%, 25%, 30%, 35%, 40%, 45%, 50% or more cells in a population of HCT-116 DS-B cells than in a population of HCT-116 cells cultured in classical conditions such as, e.g., in 10% FBS, and not subjected to any of (i) a metabolic stress, and (ii) a chemical stress applied in vitro (e.g., an in vitro exposition to about 31.5 nM, 100 nM or 315 nM SN-38 (7-ethyl-10-hydroxy-camptothecin) for a period of about 48 hours); and/or than in a population of HCT-116 cells subjected to a metabolic stress, e.g., cultured in 2% FBS but not subjected to a chemical stress applied in vitro (e.g., an in vitro exposition to about 31.5 nM, 100 nM or 315 nM SN-38 (7-ethyl-10-hydroxy-camptothecin) for a period of about 48 hours). Additionally or alternatively, the mean fluorescence intensity (MFI) for the marker CD66 (CEA) is at least twice higher, preferably at least 3 times, 4 times, 5 times, 6 times or more higher in a population of HCT-116 DS-B cells than in a population of HCT-116 cells cultured in classical conditions such as, e.g., in 10% FBS, and not subjected to any of (i) a metabolic stress, and (ii) a chemical stress applied in vitro (e.g., an in vitro exposition to about 31.5 nM, 100 nM or 315 nM SN-38 (7-ethyl-10-hydroxy-camptothecin) for a period of about 48 hours); and/or than in a population of HCT-116 cells subjected to a metabolic stress, e.g., cultured in 2% FBS but not subjected to a chemical stress applied in vitro (e.g., an in vitro exposition to about 31.5 nM, 100 nM or 315 nM SN-38 (7-ethyl-10-hydroxy-camptothecin) for a period of about 48 hours).

In one embodiment, HCT-116 DS-B cells overexpress at least one or several of the following proteins, as compared to HCT-116 cells subjected to a metabolic stress, e.g., cultured in 2% FBS but not subjected to a chemical stress applied in vitro: beta-2-glycoprotein 1, histone H2A type 1, poly(rC)-binding protein 2, kinectin, plectin, and NADH dehydrogenase [ubiquinone] 1 alpha subcomplex subunit 7.

In one embodiment, HCT-116 DS-B cells specifically express, to detectable levels, at least one or several of the following proteins, as compared to HCT-116 cells subjected to a metabolic stress, e.g., cultured in 2% FBS but not subjected to a chemical stress applied in vitro: beta-2-glycoprotein 1, histone H2A type 1, and poly(rC)-binding protein 2.

In one embodiment, HCT-116 DS-B cells overexpress at least one or several of the following membrane and/or cell surface proteins, as compared to HCT-116 cells subjected to a metabolic stress, e.g., cultured in 2% FBS but not subjected to a chemical stress applied in vitro: mitochondrial phosphate carrier protein, integrin beta-4, lysosome-associated membrane glycoprotein 1, CD44 antigen, ribosome-binding protein 1, protein transport protein Sec61 subunit alpha isoform 1, kinectin, HLA class I histocompatibility antigen A alpha chain, lysophospholipid acyltransferase 7, membrane-associated progesterone receptor component 1, microsomal glutathione S-transferase 1, NADH dehydrogenase [ubiquinone] 1 beta subcomplex subunit 4, desmoglein-2, integrin alpha-3, torsin-1A-interacting protein 1, plasma membrane calcium-transporting ATPase 1, sphingosine-1-phosphate lyase 1, V-type proton ATPase 116 kDa subunit al, mitochondrial inner membrane protein OXA1L, mitochondrial NADH dehydrogenase [ubiquinone] 1 beta subcomplex subunit 5, transmembrane protein 43, amine oxidase [flavin-containing] B, protein transport protein Sec61 subunit beta, secretory carrier-associated membrane protein 3, protein FAM162A, retinol dehydrogenase 11, ADP-ribosylation factor-like protein 8B, NADH dehydrogenase [ubiquinone] 1 beta subcomplex subunit 3, dolichol-phosphate mannosyltransferase subunit 3, mitochondrial thiamine pyrophosphate carrier, and ORM1-like protein 2.

In one embodiment, the intermediate composition comprises or consists of (i) stressed LoVo cells and (ii) stress and/or resistance proteins produced by these LoVo cells in response to
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro exposition to at least one or several chemotherapeutic agents and/or alcohols, for a period ranging from about 6 hours to about 120 hours, preferably from about 24 hours to about 96 hours, such as, e.g., 24, 30, 36, 42, 48, 54, 60, 66, 72, 78, 84, 90, or 96 hours.

In one embodiment, the intermediate composition comprises or consists of (i) stressed LoVo cells and (ii) stress and/or resistance proteins produced by these LoVo cells in response to
(i) an in vitro culture in low serum culture conditions in a 2% FBS culture medium, and
(ii) an in vitro exposition to about 5 μM fluorouracil (5-FU) for a period of about 48 hours.

This intermediate composition is herein referred to as “LoVo DS-B”.

In one embodiment, LoVo DS-B comprises stress and/or resistance proteins selected from the group comprising or consisting of radiation resistance proteins, thermal stress resistance proteins, metabolic stress resistance proteins, and combination thereof.

In one embodiment, LoVo DS-B cells overexpress at least one or the two following markers: CD243 (MDR-1) and CD66 (CEA); preferably LoVo DS-B cells overexpress CD243 (MDR-1).

In one embodiment, LoVo DS-B cells express at least 10% more, preferably at least 15%, 20%, 25%, 30%, 35%, 40%, 45%, 50% or more of at least one or the two markers CD243 (MDR-1) and CD66 (CEA)—preferably the marker CD243 (MDR-1)—as compared to LoVo cells cultured in classical conditions such as, e.g., in 10% FBS, and not subjected to any of (i) a metabolic stress, and (ii) a chemical stress applied in vitro (e.g., an in vitro exposition to about 5 μM fluorouracil (5-FU) for a period of about 48 hours); and/or as compared to LoVo cells subjected to a metabolic stress, e.g., cultured in 2% FBS but not subjected to a chemical stress applied in vitro (e.g., an in vitro exposition to about 5 μM fluorouracil (5-FU) for a period of about 48 hours). Additionally or alternatively, at least one or the two markers CD243 (MDR-1) and CD66 (CEA)—preferably the marker CD243 (MDR-1)—are expressed in at least 10% more, preferably at least 15%, 20%, 25%, 30%, 35%, 40%, 45%, 50% or more cells in a population of LoVo DS-B cells than in a population of LoVo cells cultured in classical conditions such as, e.g., in 10% FBS, and not subjected to any of (i) a metabolic stress, and (ii) a chemical stress applied in vitro (e.g., an in vitro exposition to about 5 μM fluorouracil (5-FU) for a period of about 48 hours); and/or than in a population of LoVo cells subjected to a metabolic stress, e.g., cultured in 2% FBS but not subjected to a chemical stress applied in vitro (e.g., an in vitro exposition to about 5 μM fluorouracil (5-FU) for a period of about 48 hours). Additionally or alternatively, the mean fluorescence intensity (MFI) for at least one or the two markers CD243 (MDR-1) and CD66 (CEA)—preferably the marker CD243 (MDR-1)—is at least twice higher, preferably at least 3 times, 4 times, 5 times, 6 times or more higher in a population of LoVo DS-B cells than in a population of LoVo cells cultured in classical conditions such as, e.g., in 10% FBS, and not subjected to any of (i) a metabolic stress, and (ii) a chemical stress applied in vitro (e.g., an in vitro exposition to about 5 μM fluorouracil (5-FU) for a period of about 48 hours); and/or than in a population of LoVo cells subjected to a metabolic stress, e.g., cultured in 2% FBS but not subjected to a chemical stress applied in vitro (e.g., an in vitro exposition to about 5 μM fluorouracil (5-FU) for a period of about 48 hours).

In one embodiment, LoVo DS-B cells overexpress at least one or several of the following proteins, as compared to LoVo cells subjected to a metabolic stress, e.g., cultured in 2% FBS but not subjected to a chemical stress applied in vitro: beta-2-glycoprotein 1, histone H2A type 1, poly(rC)-binding protein 2, kinectin, plectin, and NADH dehydrogenase [ubiquinone] 1 alpha subcomplex subunit 7.

In one embodiment, LoVo DS-B cells specifically express, to detectable levels, at least one or several of the following proteins, as compared to LoVo cells subjected to a metabolic stress, e.g., cultured in 2% FBS but not subjected to a chemical stress applied in vitro: beta-2-glycoprotein 1, histone H2A type 1, and poly(rC)-binding protein 2.

In one embodiment, LoVo DS-B cells overexpress at least one or several of the following membrane and/or cell surface proteins, as compared to LoVo cells subjected to a metabolic stress, e.g., cultured in 2% FBS but not subjected to a chemical stress applied in vitro: nuclear pore membrane glycoprotein 210, reticulon-4, NADPH-cytochrome P450 reductase, long-chain-fatty-acid-CoA ligase 3, long-chain-fatty-acid—CoA ligase 4, vesicle-associated membrane protein-associated protein A, mitochondrial tricarboxylate transport protein, kinectin, vesicle-associated membrane protein-associated protein B/C, very-long-chain (3R)-3-hydroxyacyl-CoA dehydratase 3, vesicular integral-membrane protein VIP36, very-long-chain enoyl-CoA reductase, microsomal glutathione S-transferase 1, cytochrome b5 type B, mitochondrial ATP synthase membrane subunit DAPIT, mitochondrial ATP synthase subunit f, cytochrome c oxidase subunit 2, mitochondrial heme protein cytochrome ci, HLA class I histocompatibility antigen B alpha chain, protein LYRIC, monocarboxylate transporter 1, cytochrome c oxidase subunit NDUFA4, mannosyl-oligosaccharide glucosidase, signal peptidase complex catalytic subunit SEC11A, protein FAM162A, transmembrane emp24 domain-containing protein 2, retinol dehydrogenase 11, erlin-1, BRI3-binding protein, 3-beta-hydroxysteroid-Delta(8), Delta(7)-isomerase, receptor expression-enhancing protein 5, CDGSH iron-sulfur domain-containing protein 2, CD9 antigen, extended synaptotagmin-2, very long-chain acyl-CoA synthetase, Kunitz-type protease inhibitor 2, CD166 antigen, long-chain-fatty-acid—CoA ligase 1, leukocyte surface antigen CD47, tapasin, and beta-1,3-galactosyl-O-glycosyl-glycoprotein beta-1,6-N-acetylglucosaminyltransferase 3.

### Method of Manufacturing

The present invention also relates to a method of manufacturing the intermediate compositions described above.

In one embodiment, the method of manufacturing the intermediate compositions described above comprises the following steps:
a) cultivating HT-29, HCT-116 or LoVo cells in a suitable culture medium;
b) subjecting the HT-29, HCT-116 or LoVo cells cultured in step a) to one or several stress[es] in vitro, wherein these HT-29, HCT-116 or LoVo cells develop resistance mechanism[s] in response to the one or several stress[es] and thereby produce stress and/or resistance proteins, and
c) recovering the stressed HT-29, HCT-116 or LoVo cells together with the stress and/or resistance proteins they have produced in step b).

In one embodiment, step a) is carried out in a depleted medium (e.g., partially or totally lacking one or several substances that are useful or even essential for the growth and/or survival of cancer cells), under hypoxia, and/or at low pH (e.g., below pH 6.5). In one embodiment, step a) is carried out in low serum culture conditions in a 2% FBS culture medium. In one embodiment, this culture in a depleted medium is maintained during step b).

Stresses applied at step b) have been described above.

In one embodiment, step c) is carried out at least several hours after the completion of step b), preferably more than 12, 24, 36, 48, 60, 72, 84, 96 hours or more after the completion of step b). This ensures that the HT-29, HCT-116 or LoVo cells have had sufficient time to develop their resistance mechanism[s] in response to the one or several stress[es] and thus, sufficient time to produce stress and/or resistance proteins.

In one embodiment, the method further comprises a step d) of treating the stressed HT-29, HCT-116 or LoVo cells and the stress and/or resistance proteins they have produced, all together recovered in step c), with a molecule or by a means capable of rendering the stress and/or resistance proteins immunogenic.

In one embodiment, step d) comprises or consists of linking or complexing the stress and/or resistance proteins to/with a means capable to confer immunogenicity.

Means capable to confer immunogenicity are well known to the skilled artisan. In one embodiment, the means capable to confer immunogenicity comprise or consist of one or several molecule[s] that is/are not naturally present in the HT-29, HCT-116 and LoVo cells or in their environment. Such means have been detailed above, and include, but are not limited to, haptens.

Examples of haptens include, but are not limited to, 2,4-dinitrophenyl (DNP); 2,4-dinitrofluorobenzene; sulfanilic acid; N-iodoacetyl-N′-(5-sulfonic-naphthyl)ethylene diamine; anilin; p-amino benzoic acid; biotin; fluorescein and derivatives thereof (including FITC, TAMRA, and Texas Red); digoxigenin; 5-nitro-3-pyrazolecarbamide; 4,5-dimethoxy-2-nitrocinnamide; 2-(3,4-dimethoxyphenyl)-quinoline-4-carbamide; 2,1,3-benzoxadiazole-5-carbamide; 3-hydroxy-2-quinoxalinecarbamide, 4-(dimethylamino)azobenzene-4′-sulfonamide (DABSYL); rotenone isooxazoline; (E)-2-(2-(2-oxo-2,3-dihydro-1H-benzo[b][1,4]diazepin-4-yl)phenozy)acetamide; 7-(diethylamino)-2-oxo-2H-chromene-3-carboxylic acid; 2-acetamido-4-methyl-5-thiazolesulfonamide; and p-methoxyphenylpyrazopodophyllamide.

In one embodiment, step d) comprises or consists of haptenating the stress and/or resistance proteins. In one embodiment, step d) comprises or consists of 2,4-dinitrophenylating the stress and/or resistance proteins (i.e., haptenating the stress and/or resistance proteins with 2,4-dinitrophenyl).

The skilled artisan is well aware of means and methods to haptenize proteins such as stress and/or resistance proteins.

In one embodiment where the intermediate compositions further comprise tumor-associated antigens (TAA) and/or tumor-specific antigens (TSA), specific of the HT-29, HCT-116 or LoVo cells, step d) also comprises linking or complexing these TAA and/or TSA to/with a means capable to confer immunogenicity, in the same conditions as described above for stress and/or resistance proteins.

In one embodiment, the method further comprises a step of inactivating the HT-29, HCT-116 or LoVo cells in order to render them non-proliferative. If applicable, this step occurs at any time after the HT-29, HCT-116 or LoVo cells have developed their resistance mechanism[s] in response to the stress[es] applied in vitro and therefore, after the HT-29, HCT-116 or LoVo cells have produced stress and/or resistance proteins. In one embodiment, the step of inactivating the HT-29, HCT-116 or LoVo cells is carried out at least several hours after the completion of step b), preferably more than 12, 24, 36, 48, 60, 72, 84, 96 hours or more after the completion of step b). In one embodiment, the step of inactivating the HT-29, HCT-116 or LoVo cells is carried out after the completion of step c). In one embodiment, the step of inactivating the HT-29, HCT-116 or LoVo cells is carried out after the completion of step d), if applicable.

The skilled artisan is well aware of means and methods for rendering cells non-proliferative. By way of examples, cells can be rendered non-proliferative by radiation with a dose sufficiently high to kill or inactivate the cells. In one embodiment, radiation—to render the cells non-proliferative—comprises or consists of irradiating the cells with a total dose of 25 Gy or above. Alternatively or additionally, cells can be rendered non-proliferative by ethanol fixation, e.g., using from about 10% to about 50% v/v of ethanol. Alternatively or additionally, cells can be rendered non-proliferative by at least one freeze-thaw cycle. Alternatively or additionally, cells can be rendered non-proliferative by linkage to or by complexation with a means capable to confer immunogenicity, such as, e.g., by haptenation.

The skilled artisan is also familiar with means and methods to determine whether a cell is non-proliferative or not, e.g., by carrying out viability tests by cell culture (to assess the total lack of proliferation) and/or propidium iodide (which distinguishes between living cells and dead cells).

In one embodiment, the method is for manufacturing the intermediate composition “HT-29 DS-A” comprising or consisting of (i) stressed HT-29 cells, and (ii) stress and/or resistance proteins, wherein the stress and/or resistance proteins were produced by these HT-29 cells in response to (i) a metabolic stress, (ii) radiations and (iii) a thermal stress applied in vitro.

In this embodiment, step b) of the method comprises or consists of subjecting the HT-29 cells cultured in step a) of the method to the following stresses in vitro: a metabolic stress, radiations and a thermal stress. Radiations, thermal stresses and metabolic stresses have been described above.

In this embodiment, step b) of the method comprises or consists of subjecting the HT-29 cells cultured in step a) of the method to the following stresses in vitro:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro radiation with a total dose ranging from about 0.25 to about 25 Gy, preferably from about 1 to about 15 Gy, such as, e.g., with a total dose of 1, 2, 3, 4, 5, 6, 7, 8, 9, 10, 11, 12, 13, 14, or 15 Gy. In one embodiment, the irradiation period ranges from about 1 to about 20 minutes, such as, e.g., 1, 2, 3, 4, 5, 6, 7, 8, 9, 10, 11, 12, 13, 14, 15, 16, 17, 18, 19, or 20 minutes, preferably from about 1 to about 5 minutes; and
(iii) an in vitro thermic choc at a temperature ranging from about 38° C. to about 45° C., such as, e.g., at a temperature of about 38° C., 39° C., 40° C., 41° C., 42° C., 43° C., 44° C., or 45° C., applied to the cells for a period ranging from about 15 minutes to about 4 hours, preferably from about 30 minutes to about 2 hours, such as, e.g., 30, 35, 40, 45, 50, 55, 60, 65, 70, 75, 80, 85, 90, 95, 100, 105, 110, 115, or 120 minutes.

In this embodiment, step b) of the method comprises or consists of subjecting the HT-29 cells cultured in step a) of the method to the following stresses in vitro:
(i) an in vitro culture in low serum culture conditions in a 2% FBS culture medium,
(ii) an in vitro radiation with a total dose of 10 Gy for a period of about 1 to about 5 minutes, and
(iii) an in vitro thermic choc at a temperature of about 42° C. for a period of about 60 minutes.

In one embodiment, the stress under (i) is applied throughout the whole period of step b). In one embodiment, the stress under (iii) is applied immediately or directly after the stress under (ii), such as, e.g., less than 10 minutes, 20 minutes, 30 minutes, 45 minutes, 1 hour, 2 hours, or 3 hours after the end of the stress under (ii), while maintaining the stress under (i).

In one embodiment, the method is for manufacturing the intermediate composition “HCT-116 DS-A” comprising or consisting of (i) stressed HCT-116 cells, and (ii) stress and/or resistance proteins, wherein the stress and/or resistance proteins were produced by these HCT-116 cells in response to (i) a metabolic stress, (ii) radiations and (iii) a thermal stress applied in vitro.

In this embodiment, step b) of the method comprises or consists of subjecting the HCT-116 cells cultured in step a) of the method to the following stresses in vitro: a metabolic stress, radiations and a thermal stress. Radiations, thermal stresses and metabolic stresses have been described above.

In this embodiment, step b) of the method comprises or consists of subjecting the HCT-116 cells cultured in step a) of the method to the following stresses in vitro:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro radiation with a total dose ranging from about 0.25 to about 25 Gy, preferably from about 1 to about 15 Gy, such as, e.g., with a total dose of 1, 2, 3, 4, 5, 6, 7, 8, 9, 10, 11, 12, 13, 14, or 15 Gy. In one embodiment, the irradiation period ranges from about 1 to about 20 minutes, such as, e.g., 1, 2, 3, 4, 5, 6, 7, 8, 9, 10, 11, 12, 13, 14, 15, 16, 17, 18, 19, or 20 minutes, preferably from about 1 to about 5 minutes; and
(iii) an in vitro thermic choc at a temperature ranging from about 38° C. to about 45° C., such as, e.g., at a temperature of about 38° C., 39° C., 40° C., 41° C., 42° C., 43° C., 44° C., or 45° C., applied to the cells for a period ranging from about 15 minutes to about 4 hours, preferably from about 30 minutes to about 2 hours, such as, e.g., 30, 35, 40, 45, 50, 55, 60, 65, 70, 75, 80, 85, 90, 95, 100, 105, 110, 115, or 120 minutes.

In this embodiment, step b) of the method comprises or consists of subjecting the HCT-116 cells cultured in step a) of the method to the following stresses in vitro:
(i) an in vitro culture in low serum culture conditions in a 2% FBS culture medium,
(ii) an in vitro radiation with a total dose of 10 Gy for a period of about 1 to about 5 minutes, and
(iii) an in vitro thermic choc at a temperature of about 42° C. for a period of about 60 minutes.

In one embodiment, the stress under (i) is applied throughout the whole period of step b). In one embodiment, the stress under (iii) is applied immediately or directly after the stress under (ii), such as, e.g., less than 10 minutes, 20 minutes, 30 minutes, 45 minutes, 1 hour, 2 hours, or 3 hours after the end of the stress under (ii), while maintaining the stress under (i).

In one embodiment, the method is for manufacturing the intermediate composition “LoVo DS-A” comprising or consisting of (i) stressed LoVo cells, and (ii) stress and/or resistance proteins, wherein the stress and/or resistance proteins were produced by these LoVo cells in response to (i) a metabolic stress, (ii) radiations and (iii) a thermal stress applied in vitro.

In this embodiment, step b) of the method comprises or consists of subjecting the LoVo cells cultured in step a) of the method to the following stresses in vitro: a metabolic stress, radiations and a thermal stress. Radiations, thermal stresses and metabolic stresses have been described above.

In this embodiment, step b) of the method comprises or consists of subjecting the LoVo cells cultured in step a) of the method to the following stresses in vitro:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro radiation with a total dose ranging from about 0.25 to about 25 Gy, preferably from about 1 to about 15 Gy, such as, e.g., with a total dose of 1, 2, 3, 4, 5, 6, 7, 8, 9, 10, 11, 12, 13, 14, or 15 Gy. In one embodiment, the irradiation period ranges from about 1 to about 20 minutes, such as, e.g., 1, 2, 3, 4, 5, 6, 7, 8, 9, 10, 11, 12, 13, 14, 15, 16, 17, 18, 19, or 20 minutes, preferably from about 1 to about 5 minutes; and
(iii) an in vitro thermic choc at a temperature ranging from about 38° C. to about 45° C., such as, e.g., at a temperature of about 38° C., 39° C., 40° C., 41° C., 42° C., 43° C., 44° C., or 45° C., applied to the cells for a period ranging from about 15 minutes to about 4 hours, preferably from about 30 minutes to about 2 hours, such as, e.g., 30, 35, 40, 45, 50, 55, 60, 65, 70, 75, 80, 85, 90, 95, 100, 105, 110, 115, or 120 minutes.

In this embodiment, step b) of the method comprises or consists of subjecting the LoVo cells cultured in step a) of the method to the following stresses in vitro:
(i) an in vitro culture in low serum culture conditions in a 2% FBS culture medium,
(ii) an in vitro radiation with a total dose of 10 Gy for a period of about 1 to about 5 minutes, and
(iii) an in vitro thermic choc at a temperature of about 42° C. for a period of about 60 minutes.

In one embodiment, the stress under (i) is applied throughout the whole period of step b). In one embodiment, the stress under (iii) is applied immediately or directly after the stress under (ii), such as, e.g., less than 10 minutes, 20 minutes, 30 minutes, 45 minutes, 1 hour, 2 hours, or 3 hours after the end of the stress under (ii), while maintaining the stress under (i).

In one embodiment, the method is for manufacturing the intermediate composition “HT-29 DS-B” comprising or consisting of (i) stressed HT-29 cells, and (ii) stress and/or resistance proteins, wherein the stress and/or resistance proteins were produced by these HT-29 cells in response to (i) a metabolic stress, and (ii) a chemical stress applied in vitro.

In this embodiment, step b) of the method comprises or consists of subjecting the HT-29 cells cultured in step a) of the method to the following stresses in vitro: a metabolic stress and a chemical stress. Chemical stresses and metabolic stresses have been described above.

In this embodiment, step b) of the method comprises or consists of subjecting the HT-29 cells cultured in step a) of the method to the following stresses in vitro:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro exposition to at least one or several chemotherapeutic agents and/or alcohols, for a period ranging from about 6 hours to about 120 hours, preferably from about 24 hours to about 96 hours, such as, e.g., 24, 30, 36, 42, 48, 54, 60, 66, 72, 78, 84, 90, or 96 hours.

In this embodiment, step b) of the method comprises or consists of subjecting the HT-29 cells cultured in step a) of the method to the following stresses in vitro:
(i) an in vitro culture in low serum culture conditions in a 2% FBS culture medium,
(ii) an in vitro exposition to about 13 μM oxaliplatin for a period of about 72 hours.

In one embodiment, the stress under (i) is applied throughout the whole period of step b). In one embodiment, the stress under (ii) is applied while maintaining the stress under (i).

In one embodiment, the method is for manufacturing the intermediate composition “HCT-116 DS-B” comprising or consisting of (i) stressed HCT-116 cells, and (ii) stress and/or resistance proteins, wherein the stress and/or resistance proteins were produced by these HCT-116 cells in response to (i) a metabolic stress, and (ii) a chemical stress applied in vitro.

In this embodiment, step b) of the method comprises or consists of subjecting the HCT-116 cells cultured in step a) of the method to the following stresses in vitro: a metabolic stress and a chemical stress. Chemical stresses and metabolic stresses have been described above.

In this embodiment, step b) of the method comprises or consists of subjecting the HCT-116 cells cultured in step a) of the method to the following stresses in vitro:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro exposition to at least one or several chemotherapeutic agents and/or alcohols, for a period ranging from about 6 hours to about 120 hours, preferably from about 24 hours to about 96 hours, such as, e.g., 24, 30, 36, 42, 48, 54, 60, 66, 72, 78, 84, 90, or 96 hours.

In this embodiment, step b) of the method comprises or consists of subjecting the HCT-116 cells cultured in step a) of the method to the following stresses in vitro:
(i) an in vitro culture in low serum culture conditions in a 2% FBS culture medium,
(ii) an in vitro exposition to about 31.5 nM, 100 nM or 315 nM SN-38 (7-ethyl-10-hydroxy-camptothecin) for a period of about 48 hours.

In one embodiment, the stress under (i) is applied throughout the whole period of step b).

In one embodiment, the stress under (ii) is applied while maintaining the stress under (i).

In one embodiment, the method is for manufacturing the intermediate composition “LoVo DS-B” comprising or consisting of (i) stressed LoVo cells, and (ii) stress and/or resistance proteins, wherein the stress and/or resistance proteins were produced by these LoVo cells in response to (i) a metabolic stress, and (ii) a chemical stress applied in vitro.

In this embodiment, step b) of the method comprises or consists of subjecting the LoVo cells cultured in step a) of the method to the following stresses in vitro: a metabolic stress and a chemical stress. Chemical stresses and metabolic stresses have been described above.

In this embodiment, step b) of the method comprises or consists of subjecting the LoVo cells cultured in step a) of the method to the following stresses in vitro:
(i) an in vitro culture in a depleted medium (e.g., partially or totally lacking one or several substances that are useful or even essential for the growth and/or survival of cancer cells), under hypoxia, and/or at low pH (e.g., below pH 6.5),
(ii) an in vitro exposition to at least one or several chemotherapeutic agents and/or alcohols, for a period ranging from about 6 hours to about 120 hours, preferably from about 24 hours to about 96 hours, such as, e.g., 24, 30, 36, 42, 48, 54, 60, 66, 72, 78, 84, 90, or 96 hours.

In this embodiment, step b) of the method comprises or consists of subjecting the LoVo cells cultured in step a) of the method to the following stresses in vitro:
(i) an in vitro culture in low serum culture conditions in a 2% FBS culture medium,
(ii) an in vitro exposition to about 5 μM fluorouracil (5-FU) for a period of about 48 hours.

In one embodiment, the stress under (i) is applied throughout the whole period of step b). In one embodiment, the stress under (ii) is applied while maintaining the stress under (i).

The present invention also relates to a method of manufacturing the composition comprising or consisting of (i) stressed HT-29, HCT-116 and LoVo cells, and (ii) immunogenic stress and/or resistance proteins, described above. This final composition is herein referred to as “DP”.

In one embodiment, the method of manufacturing the composition comprising or consisting of (i) stressed HT-29, HCT-116 and LoVo cells, and (ii) immunogenic stress and/or resistance proteins, described above, comprises the following steps:
a) obtaining the HT-29 DS-A, HCT-116 DS-A, LoVo DS-A, HT-29 DS-B, HCT-116 DS-B and LoVo DS-B intermediate compositions using the method of manufacturing the intermediate compositions described above; and
b) mixing the HT-29 DS-A, HCT-116 DS-A, LoVo DS-A, HT-29 DS-B, HCT-116 DS-B and LoVo DS-B intermediate compositions together.

In one embodiment, step b) comprises mixing the six intermediate compositions to a final concentration of about 10⁶ to about 10⁹ stressed HT-29, HCT-116 and LoVo cells per mL of composition, preferably from about 10⁷ to about 10⁸ stressed HT-29, HCT-116 and LoVo cells per mL of composition, such as, about 1×10⁷, 2×10⁷, 3×10⁷, 4×10⁷, 5×10⁷, 6×10⁷, 7×10⁷, 8×10⁷, 9×10⁷, or 1×10⁸ stressed HT-29, HCT-116 and LoVo cells per mL of composition. In one embodiment, step b) comprises mixing the six intermediate compositions to a final concentration of about 3×10⁷ stressed HT-29, HCT-116 and LoVo cells per mL of composition.

In one embodiment, step b) comprises mixing the six intermediate compositions in an equal ratio of stressed HT-29, HCT-116 and LoVo cells (i.e., about 1:1:1:1:1:1). In one embodiment, step b) comprises mixing the six intermediate compositions in a ratio comprising or consisting of at least 1.5, 2 or 2.5 times more stressed HT-29 than stressed HCT-116 or LoVo cells. In one embodiment, step b) comprises mixing the six intermediate compositions in a ratio comprising or consisting of at least 1.5, 2 or 2.5 times more stressed HCT-116 than stressed HT-29 or LoVo cells. In one embodiment, step b) comprises mixing the six intermediate compositions in a ratio comprising or consisting of at least 1.5, 2 or 2.5 times more stressed LoVo than stressed HT-29 or HCT-116 cells.

In one embodiment, step b) comprises mixing together:
from about 10⁵ to about 10⁸ stressed HT-29 cells (from HT-29 DS-A and HT-29 DS-B), preferably from about 10⁶ to about 10⁷ stressed HT-29 cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed HT-29 cells,
from about 10⁵ to about 10⁸ stressed HCT-116 cells (from HCT-116 DS-A and HCT-116 DS-B), preferably from about 10⁶ to about 10⁷ stressed HCT-116 cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed HCT-116 cells, and
from about 10⁵ to about 10⁸ stressed LoVo cells (from LoVo DS-A and LoVo DS-B), preferably from about 10⁶ to about 107 stressed LoVo cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed LoVo cells.

### Therapeutic Uses and Methods

The present invention also relates to a method of treating cancer in a subject in need thereof, comprising administering to said subject the composition comprising or consisting of (i) stressed HT-29, HCT-116 and LoVo cells, and (ii) immunogenic stress and/or resistance proteins produced by these cells in response to a stress applied in vitro, as defined hereinabove.

The present invention also relates to the composition comprising or consisting of (i) stressed HT-29, HCT-116 and LoVo cells, and (ii) immunogenic stress and/or resistance proteins produced by these cells in response to a stress applied in vitro, as defined hereinabove, for use in treating cancer in a subject in need thereof.

The present invention also relates to the use of the composition comprising or consisting of (i) stressed HT-29, HCT-116 and LoVo cells, and (ii) immunogenic stress and/or resistance proteins produced by these cells in response to a stress applied in vitro, as defined hereinabove, for treating cancer in a subject in need thereof.

The present invention also relates to the use of the composition comprising or consisting of (i) stressed HT-29, HCT-116 and LoVo cells, and (ii) immunogenic stress and/or resistance proteins produced by these cells in response to a stress applied in vitro, as defined hereinabove, for the manufacture of a medicament for treating cancer in a subject in need thereof.

In one embodiment, treating cancer comprises eliciting an immune response against cancer cells from said cancer.

Examples of cancers include those listed in the 10^{th} revision of the International Statistical Classification of Diseases and Related Health Problems (ICD), under chapter II, blocks C00 to D48.

Further examples of cancers include, but are not limited to, recurrent, metastatic or multi-drug resistant cancer.

Further examples of cancers include, but are not limited to, adenofibroma, adenoma, agnogenic myeloid metaplasia, AIDS-related malignancies, ameloblastoma, anal cancer, angiofollicular mediastinal lymph node hyperplasia, angiokeratoma, angiolymphoid hyperplasia with eosinophilia, angiomatosis, anhidrotic ectodermal dysplasia, anterofacial dysplasia, apocrine metaplasia, apudoma, asphyxiating thoracic dysplasia, astrocytoma (including, e.g., cerebellar astrocytoma and cerebral astrocytoma), atriodigital dysplasia, atypical melanocytic hyperplasia, atypical metaplasia, autoparenchymatous metaplasia, basal cell hyperplasia, benign giant lymph node hyperplasia, bile duct cancer (including, e.g., extrahepatic bile duct cancer), bladder cancer, bone cancer, brain tumor (including, e.g., brain stem glioma, cerebellar astrocytoma glioma, malignant glioma, supratentorial primitive neuroectodermal tumors, visual pathway and hypothalamic glioma, ependymoma, medulloblastoma, gestational trophoblastic tumor glioma, and paraganglioma), branchionia, female breast cancer, male breast cancer, bronchial adenomas/carcinoids, bronchopulmonary dysplasia, cancer growths of epithelial cells, pre-cancerous growths of epithelial cells, metastatic growths of epithelial cells, carcinoid heart disease, carcinoid tumor (including, e.g., gastrointestinal carcinoid tumor), carcinoma (including, e.g., carcinoma of unknown primary origin, adrenocortical carcinoma, islet cells carcinoma, adeno carcinoma, adeoncortical carcinoma, basal cell carcinoma, basosquamous carcinoma, bronchiolar carcinoma, Brown-Pearce carcinoma, cystadenocarcinoma, ductal carcinoma, hepatocarcinoma, Krebs carcinoma, papillary carcinoma, oat cell carcinoma, small cell lung carcinoma, non-small cell lung carcinoma, squamous cell carcinoma, transitional cell carcinoma, Walker carcinoma, Merkel cell carcinoma, and skin carcinoma), cementoma, cementum hyperplasia, cerebral dysplasia, cervical cancer, cervical dysplasia, cholangioma, cholesteatoma, chondroblastoma, chondroectodermal dysplasia, chordoma, choristoma, chrondroma, cleidocranial dysplasia, colon adenocarcinoma, colon cancer, colon carcinoma, colorectal adenocarcinoma, colorectal cancer, colorectal carcinoma, local metastasized colorectal cancer, congenital adrenal hyperplasia, congenital ectodermal dysplasia, congenital sebaceous hyperplasia, connective tissue metaplasia, craniocarpotarsal dysplasia, craniodiaphysial dysplasia, craniometaphysial dysplasia, craniopharyngioma, cylindroma, cystadenoma, cystic hyperplasia (including, e.g., cystic hyperplasia of the breast), cystosarconia phyllodes, dentin dysplasia, denture hyperplasia, diaphysial dysplasia, ductal hyperplasia, dysgenninoma, dysplasia epiphysialis hemimelia, dysplasia epiphysialis multiplex, dysplasia epiphysialis punctate, ectodermal dysplasia, Ehrlich tumor, enamel dysplasia, encephaloophthalmic dysplasia, endometrial cancer (including, e.g., ependymoma and endometrial hyperplasia), ependymoma, epithelial cancer, epithelial dysplasia, epithelial metaplasia, esophageal cancer, Ewing's family of tumors (including, e.g., Ewing's sarcoma), extrahepatic bile duct cancer, eye cancer (including, e.g., intraocular melanoma and retinoblastoma), faciodigitogenital dysplasia, familial fibrous dysplasia of jaws, familial white folded dysplasia, fibroma, fibromuscular dysplasia, fibromuscular hyperplasia, fibrous dysplasia of bone, florid osseous dysplasia, focal epithelial hyperplasia, gall bladder cancer, ganglioneuroma, gastric cancer (including, e.g., stomach cancer), gastrointestinal carcinoid tumor, gastrointestinal tract cancer, gastrointestinal tumors, Gaucher's disease, germ cell tumors (including, e.g., extracranial germ cell tumors, extragonadal germ cell tumors, and ovarian germ cell tumors), giant cell tumor, gingival hyperplasia, glioblastoma, glomangioma, granulosa cell tumor, gynandroblastoma, hamartoma, head and neck cancer, hemangioendothelioma, hemangioma, hemangiopericytoma, hepatocellular cancer, hepatoma, hereditary renal-retinal dysplasia, hidrotic ectodermal dysplasia, histiocytonia, histiocytosis, hypergammaglobulinemia, hypohidrotic ectodermal dysplasia, hypopharyngeal cancer, inflammatory fibrous hyperplasia, inflammatory papillary hyperplasia, intestinal cancers, intestinal metaplasia, intestinal polyps, intraocular melanoma, intravascular papillary endothelial hyperplasia, kidney cancer, laryngeal cancer, leiomyoma, leukemia (including, e.g., acute lymphoblastic leukemia, acute lymphocytic leukemia, acute myeloid leukemia, acute myelogenous leukemia, acute hairy cell leukemia, acute B-cell leukemia, acute T-cell leukemia, acute HTLV leukemia, chronic lymphoblastic leukemia, chronic lymphocytic leukemia, chronic myeloid leukemia, chronic myelogenous leukemia, chronic hairy cell leukemia, chronic B-cell leukemia, chronic T-cell leukemia, and chronic HTLV leukemia), Leydig cell tumor, lip and oral cavity cancer, lipoma, liver cancer, lung cancer (including, e.g., small cell lung cancer and non-small cell lung cancer), lymphangiomyoma, lymphaugioma, lymphoma (including, e.g., AIDS-related lymphoma, central nervous system lymphoma, primary central nervous system lymphoma, Hodgkin's lymphoma, non-Hodgkin's lymphoma, Hodgkin's lymphoma during pregnancy, non-Hodgkin's lymphoma during pregnancy, mast cell lymphoma, B-cell lymphoma, adenolymphoma, Burkitt's lymphoma, cutaneous T-cell lymphoma, large cell lymphoma, and small cell lymphoma), lymphopenic thymic dysplasia, lymphoproliferative disorders, macroglobulinemia (including, e.g., Waldenstrom's macroglobulinemia), malignant carcinoid syndrome, malignant mesothelioma, malignant thymoma, mammary dysplasia, mandibulofacial dysplasia, medulloblastoma, meningioma, mesenchymoma, mesonephroma, mesothelioma (including, e.g., malignant mesothelioma), metaphysial dysplasia, metaplastic anemia, metaplastic ossification, metaplastic polyps, metastatic squamous neck cancer (including, e.g., metastatic squamous neck cancer with occult primary), Mondini dysplasia, monostotic fibrous dysplasia, mucoepithelial dysplasia, multiple endocrine neoplasia syndrome, multiple epiphysial dysplasia, multiple myeloma/plasma cell neoplasm, mycosis fungoides, myelodysplastic syndrome, myeloid metaplasia, myeloproliferative disorders, chronic myeloproliferative disorders, myoblastoma, myoma, myxoma, nasal cavity and paranasal sinus cancer, nasopharyngeal cancer, prostatic neoplasm, colon neoplasm, abdomen neoplasm, bone neoplasm, breast neoplasm, digestive system neoplasm, liver neoplasm, pancreas neoplasm, peritoneum neoplasm, endocrine glands neoplasm (including, e.g., adrenal neoplasm, parathyroid neoplasm, pituitary neoplasm, testicles neoplasm, ovary neoplasm, thymus neoplasm, and thyroid neoplasm), eye neoplasm, head and neck neoplasm, nervous system neoplasm (including, e.g., central nervous system neoplasm and peripheral nervous system neoplasm), lymphatic system neoplasm, pelvic neoplasm, skin neoplasm, soft tissue neoplasm, spleen neoplasm, thoracic neoplasm, urogenital tract neoplasm, neurilemmoma, neuroblastoma, neuroepithelioma, neurofibroma, neurofibromatosis, neuroma, nodular hyperplasia of prostate, nodular regenerative hyperplasia, oculoauriculovertebral dysplasia, oculodentodigital dysplasia, oculovertebral dysplasia, odontogenic dysplasia, odontoma, opthalmomandibulomelic dysplasia, oropharyngeal cancer, osteoma, ovarian cancer (including, e.g., ovarian epithelial cancer and ovarian low malignant potential tumor), pancreatic cancer (including, e.g., islet cell pancreatic cancer and exocrine pancreatic cancer), papilloma, paraganglioma, nonchromaffin paraganglioma, paranasal sinus and nasal cavity cancer, paraproteinemias, parathyroid cancer, periapical cemental dysplasia, pheochromocytoma (including, e.g., penile cancer), pineal and supratentorial primitive neuroectodermal tumors, pinealoma, pituitary tumor, plasma cell neoplasm/multiple myeloma, plasmacytoma, pleuropulmonary blastoma, polyostotic fibrous dysplasia, polyps, pregnancy cancer, pre-neoplastic disorders (including, e.g., benign dysproliferative disorders such as benign tumors, fibrocystic conditions, tissue hypertrophy, intestinal polyps, colon polyps, esophageal dysplasia, leukoplakia, keratoses, Bowen's disease, Farmer's skin, solar cheilitis, and solar keratosis), primary hepatocellular cancer, primary liver cancer, primary myeloid metaplasia, prostate cancer, pseudoachondroplastic spondyloepiphysial dysplasia, pseudoepitheliomatous hyperplasia, purpura, rectal cancer, renal cancer (including, e.g., kidney cancer, renal pelvis, ureter cancer, transitional cell cancer of the renal pelvis and ureter), reticuloendotheliosis, retinal dysplasia, retinoblastoma, salivary gland cancer, sarcomas (including, e.g., uterine sarcoma, soft tissue sarcoma, carcinosarcoma, chondrosarcoma, fibrosarcoma, hemangiosarcoma, Kaposi's sarcoma, leiomyosarcoma, liposarcoma, lymphangiosarcoma, myosarcoma, myxosarcoma, rhabdosarcoma, sarcoidosis sarcoma, osteosarcoma, Ewing sarcoma, malignant fibrous histiocytoma of bone, and clear cell sarcoma of tendon sheaths), sclerosing angioma, secondary myeloid metaplasia, senile sebaceous hyperplasia, septooptic dysplasia, Sertoli cell tumor, Sezary syndrome, skin cancer (including, e.g., melanoma skin cancer and non-melanoma skin cancer), small intestine cancer, spondyloepiphysial dysplasia, squamous metaplasia (including, e.g., squamous metaplasia of amnion), stomach cancer, supratentorial primitive neuroectodermal and pineal tumors, supratentorial primitive neuroectodermal tumors, symptomatic myeloid metaplasia, teratoma, testicular cancer, theca cell tumor, thymoma (including, e.g., malignant thymoma), thyroid cancer, trophoblastic tumors (including, e.g., gestational trophoblastic tumors), ureter cancer, urethral cancer, uterine cancer, vaginal cancer, ventriculoradial dysplasia, verrucous hyperplasia, vulvar cancer, Waldenstrom's macroglobulinemia, and Wilms' tumor.

In one embodiment, the cancer is selected from the group comprising or consisting of renal cancer, liver cancer, pancreatic cancer, endometrial cancer, colorectal cancer, ovarian cancer, breast cancer, lung cancer, head and neck cancer, cervical cancer, melanoma and glioma.

In one embodiment, the cancer is selected from the group comprising or consisting of colon and colorectal cancers, including, but not limited to, colon adenocarcinoma, colon cancer, colon carcinoma, colorectal adenocarcinoma, colorectal cancer, and colorectal carcinoma.

In one embodiment, the composition is administered or is to be administered once. In one embodiment, the composition is administered or is to be administered several times, such as 2, 3, 4, 5, 6, 7, 8, 9, 10, 11, 12, 13, 14, 15 times or more; or indefinitely until the cancer is treated.

In one embodiment, the composition is administered or is to be administered every week, every two weeks, every three weeks, every month, every two months, every three months, every six months, every year.

In one embodiment, the composition is administered or is to be administered every week, every two weeks, every three weeks, every month, preferably every week, for the first weeks, such as for the first 2, 3, 4, 5, 6, 7, 8, 9, 10 weeks or more, preferably for the first 6 to 8 weeks, as an induction or bolus regimen.

In one embodiment, the composition is administered or is to be administered every month, every two months, every three months, every six months, every year after the induction or bolus regimen, as a boost regimen.

In one embodiment, the composition is administered or is to be administered every week for the first 6 to 8 weeks; then about one month later; then about 3 months later; then every 6 months until the cancer is treated.

In one embodiment, the composition is administered or is to be administered at a total dose ranging from about 10⁵ to about 10⁸ stressed HT-29, HCT-116 and LoVo cells, preferably from about 10⁶ to about 10⁷ stressed HT-29, HCT-116 and LoVo cells, such as, about 1×10⁶, 2×10⁶, 3×10⁶, 4×10⁶, 5×10⁶, 6×10⁶, 7×10⁶, 8×10⁶, 9×10⁶, or 1×10⁷ stressed HT-29, HCT-116 and LoVo cells.

In one embodiment, the composition is administered or is to be administered as a single dose.

In one embodiment, the composition is administered or is to be administered in at least two doses, such as 2, 3, 4, 5 or more dose.

In one embodiment, the composition is administered or is to be administered at a total dose of about 3×10⁶ stressed HT-29, HCT-116 and LoVo cells. In one embodiment, the composition is administered or is to be administered at a total dose of about 6×10⁶ stressed HT-29, HCT-116 and LoVo cells. In one embodiment, the composition is administered or is to be administered in a single dose of 6×10⁶ stressed HT-29, HCT-116 and LoVo cells, or in two doses of 3×10⁶ stressed HT-29, HCT-116 and LoVo cells.

In one embodiment, the composition is administered or is to be administered in a volume ranging from about 10 μL to about 1 mL, such as, about 10 μL, 50 μL, 100 μL, 200 μL, 300 μL, 400 μL, 500 μL, 600 μL, 700 μL, 800 μL, 900 μL, or 1 mL, preferably in a volume of about 100 μL.

In one embodiment, the composition is administered or is to be administered systemically. In one embodiment, the composition is administered or is to be administered by injection, preferably by systemic injection.

Examples of systemic injections include, but are not limited to, intravenous (iv), subcutaneous (sq), intradermal (id), intramuscular (im), intraarterial, intraparenteral, intranodal, intralymphatic, intraperitoneal (ip), intracranial, intracardiac, intralesional, intraprostatic, intravaginal, intrarectal, intrathecal, intranasal, intratumoral (it), intravesicular, and perfusion.

In one embodiment, the composition is administered or is to be administered intradermally, subcutaneously, intratumorally or intravenously.

In one embodiment, the composition is administered or is to be administered intradermally.

It will be understood that other suitable routes of administration are also contemplated in the present invention, and the administration mode will ultimately be decided by the attending physician within the scope of sound medical judgment.

In one embodiment, the composition is administered or is to be administered to the subject before, concomitantly with, or after administration of at least one additional therapy.

Examples of suitable additional therapies include chemotherapeutic agents as defined above, radiation therapy and immunostimulatory agents.

Suitable examples of radiation therapies include, but are not limited to, external beam radiotherapy (such as, e.g., superficial X-rays therapy, orthovoltage X-rays therapy, megavoltage X-rays therapy, radiosurgery, stereotactic radiation therapy, cobalt therapy, electron therapy, fast neutron therapy, neutron-capture therapy, proton therapy, and the like); brachytherapy; unsealed source radiotherapy; tomotherapy; and the like.

Suitable examples of immunostimulatory agents include those described under subgroup LW3 of the Anatomical Therapeutic Chemical Classification System. Further examples of immunostimulatory agents include, but are not limited to, cytokines (such as, e.g., filgrastim, pegfilgrastim, lenograstim, molgramostim, sargramostim, ancestim, albinterferon, interferon alfa natural, interferon alfa 2a, peginterferon alfa-2a, interferon alfa 2b, peginterferon alfa-2b, interferon alfa nl, interferon alfacon-1, interferon alpha-n3, interferon beta natural, interferon beta 1a, interferon beta 1b, interferon gamma, aldesleukin, oprelvekin, and the like): immune checkpoint inhibitors (such as, e.g., inhibitors of CTLA4, PD-1, PD-L1, LAG-3, B7-H3, B7-H4, TDM3, A2AR, and/or IDO, including nivolumab, pembrolizumab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, mogamulizumab, varlilumab, avelumab, galiximab, AMP-514, AUNP 12, indoximod, NLG-919, INCB024360, and the like); toll-like receptor agonists (such as, e.g., buprenorphine, carbamazepine, ethanol, fentanyl, GS-9620, imiqimod, lefitolimod, levorphanol, methadone, morphine, (+)-morphine, morphine-3-glucuronide, oxcarbazepine, oxycodone, pethidine, resiquimod, SD-101, tapenadol, tilsotolimod, VTX-2337, glucuronoxylomannan from Cryprocccus, MALP-2 from Mycoplasma, MALP-404 from Mycoplasma, OspA from Borrelia, porin from Neisseria or Haemophilus, hsp60, hemmaglutinin, LcrV from Yersinia, bacterial flagellin, lipopolysaccharide, lipoteichoic acid, lipomannan from Mvcobacteriun, glycosylphosphatidylinositol, lysophosphatidylserine, lipophosphoglycan from Leishmania, zymosan from Saccharomyces, Pam2CGDPKHPKSF, Pam3CSK4. CpG oligodeoxynucleotides, poly(I:C) nucleic acid sequences, poly(A:U) nucleic acid sequences, double-stranded viral RNA, and the like); STING receptor agonists (such as, e.g., those described in § 0240 of WO2017100305, vadimezan, CL656, ADU-S100, 3′3′-cGAMP, 2′3′-cGAMP. ML RR-S2 CDG, ML RR-S2 cGAMP, cyclic di-GMP, DMXAA, DiABZI, and the like); CD1 ligands; growth hormone; granulocyte-macrophage colony-stimulating factor (GM-CSF); immunocyanin; pegademase: prolactin: tasonermin; female sex steroids; histamine dihydrochloride: poly ICLC; vitamin D; lentinan; plerixafor; roquinimex; mifamurtide; glatiramer acetate; thymopentin; thymosin a1; thymulin; polyinosinic:polycytidylic acid; pidotimod; Bacillus Calmette-Gudrin; melanoma vaccine; sipuleucel-T; and the like.

In one embodiment, the composition may be administered to the subject after administration of chemotherapeutic agent such as, e.g., cyclophosphamide.

Additionally or alternatively, the composition may be administered or to the subject concomitantly with administration of an immunostimulatory agent, such as, e.g., granulocyte-macrophage colony-stimulating factor (GM-CSF).

### Kit-of-Parts

The present invention also relates to a kit-of-part.

The term “kit-of-parts”, also termed “package”, “commercial package”, or “pharmaceutical package”, shall encompass an entity of physically separated components, which are intended for individual storage and/or use, but in functional relation to each other. In particular, the term “kit-of-parts” encompasses an entity of physically separated components which are to be used or administered separately, in any order, in a given time interval, typically but not necessarily ranging from hours to days, weeks or more; or concomitantly, either as an extemporaneous formulation or individually, in any order, within a time interval ranging from seconds to minutes or hours.

In one embodiment, the kit-of-parts comprises:
the intermediate composition named “HT-29 DS-A”, as defined above;
the intermediate composition named “HCT-116 DS-A”, as defined above; and
the intermediate composition named “LoVo DS-A”, as defined above.

In one embodiment, the kit-of-parts comprises:
the intermediate composition named “HT-29 DS-B”, as defined above;
the intermediate composition named “HCT-116 DS-B”, as defined above; and
the intermediate composition named “LoVo DS-B”, as defined above.

In one embodiment, the kit-of-parts comprises:
the intermediate composition named “HT-29 DS-A”, as defined above;
the intermediate composition named “HCT-116 DS-A”, as defined above;
the intermediate composition named “LoVo DS-A”, as defined above;
the intermediate composition named “HT-29 DS-B”, as defined above;
the intermediate composition named “HCT-116 DS-B”, as defined above; and
the intermediate composition named “LoVo DS-B”, as defined above.

In one embodiment, the kit-of-parts further comprises instructions for mixing the intermediate compositions, according to the method of manufacturing the composition comprising or consisting of (i) stressed HT-29, HCT-116 and LoVo cells, and (ii) immunogenic stress and/or resistance proteins, named “DP”, described above.

In one embodiment, the kit-of-parts further comprises at least one additional therapeutic agent, such as, e.g., at least one chemotherapeutic agent and/or immunostimulatory agent, as defined above.

In one embodiment, the kit-of-parts comprises:
the composition named “DP”, as defined above;
at least one additional therapeutic agent, such as, e.g., at least one chemotherapeutic agent and/or immunostimulatory agent, as defined above.

### BRIEF DESCRIPTION OF THE DRAWINGS

FIGS. 1A-B are two Venn diagrams comparing the number of identified proteins in each of the 3 human cell lines (HT-29, HCT-116 and LoVo) for untreated samples.

FIG. 1A: RCB samples.

FIG. 1B: MCB samples.

FIG. 2 is a graph showing the differential number of proteins expressed in the HCT-116 MCB, DS-A, DS-B and in the final product (DP) comprising all six DS described above (hence including the HCT-116 DS-A and DS-B), by comparison to the initial HCT-116 RCB cultured in classical conditions (with 10% FBS). “+” indicates proteins that are newly expressed, “−” indicates the number of proteins which are no longer expressed at all.

FIG. 3 is a graph showing the number of proteins over- and under-expressed in the HCT-116 MCB, DS-A and DS-B, by comparison to the initial HCT-116 RCB cultured in classical conditions (with 10% FBS).

FIG. 4 is a graph showing the differential number of proteins expressed in the HT-29 MCB, DS-A, DS-B and in the final product (DP) comprising all six DS described above (hence including the HT-29 DS-A and DS-B), by comparison to the initial HT-29 RCB cultured in classical conditions (with 10% FBS). “+” indicates proteins that are newly expressed, “−” indicates the number of proteins which are no longer expressed at all.

FIG. 5 is a graph showing the number of proteins over- and under-expressed in the HT-29 MCB, DS-A and DS-B, by comparison to the initial HT-29 RCB cultured in classical conditions (with 10% FBS).

FIG. 6 is a graph showing the differential number of proteins expressed in the LoVo MCB, DS-A, DS-B and in the final product (DP) comprising all six DS described above (hence including the LoVo DS-A and DS-B), by comparison to the initial LoVo RCB cultured in classical conditions (with 10% FBS). “+” indicates proteins that are newly expressed, “−” indicates the number of proteins which are no longer expressed at all.

FIG. 7 is a graph showing the number of proteins over- and under-expressed in the LoVo MCB, DS-A and DS-B, by comparison to the initial LoVo RCB cultured in classical conditions (with 10% FBS).

FIG. 8 is a graph showing the number of proteins identified (on the y-axis), whether membrane and/or cell surface proteins or others (i.e., any other protein for which the terms “membrane” or “cell surface” were not reported as annotations in the “cellular component” field of the Thermo Protein Center database), in several compositions of HT-29 cells: HT-29 MCB, HT-29 DS-A, and HT-29 DS-B; as well as in the drug product (DP) comprising a mix of HT-29 DS-A, HT-29 DS-B, HCT-116 DS-A, HCT-116 DS-B, LoVo DS-A and LoVo DS-B. Experiments were performed in triplicates, indicated as “#1”, “#2” and “#3”.

FIGS. 9A-D are pie charts showing the protein distribution according to p-values and fold changes, between HT-29 DS-A versus HT-29 MCB (FIG. 9A), HT-29 DS-B versus HT-29 MCB (FIG. 9B), HT-29 DS-A versus HT-29 DS-B (FIG. 9C), and DP versus HT-29 MCB (FIG. 9D).

FIG. 10 is a graph showing the number of membrane and/or cell surface proteins (on the y-axis) which are either statistically significantly under-expressed (i.e., with a p-value≤0.05 and an abundance ratio≤0.5) or over-expressed (i.e., with a p-value≤0.05 and an abundance ratio≥2) between HT-29 DS-A versus HT-29 MCB, HT-29 DS-B versus HT-29 MCB, HT-29 DS-A versus HT-29 DS-B, and DP versus HT-29 MCB. The number of under-expressed proteins is displayed as negative number.

FIGS. 11A-C are three graphs showing the secretion of IL-8 or IL-12 from dendritic cells, expressed in μg/mL. FIG. 11A: secretion of IL-8 in absence of CD40L; FIG. 11B: secretion of IL-8 in presence of 0.6 μg/mL CD40L; FIG. 11C: secretion of IL-12 in presence of 0.6 μg/mL CD40L.

FIG. 12 is a graph showing the IFNγ release in the supernatant collected from a co-culture of mDCs and autologous CD8⁺ T cells treated with various ratios of STC-1010. The hashed line represents the limit of detection of IFNγ.

FIGS. 13A-C are three graphs showing the expression of the DCs activation markers CD40, CD83 and CD86 assessed by RT-qPCR upon treatment with various doses of STC-1010. Data are expressed as relative quantity normalized to the expression of chicken GAPDH. FIG. 13A: expression of CD40; FIG. 13B: expression of CD83; FIG. 13C: expression of CD86.

### EXAMPLES

The present invention is further illustrated by the following examples.

### Example 1

### Selection of Cell Lines for the Human Colorectal Cancer Vaccine

We aimed at developing an anti-cancer treatment turning cancer cell resistance mechanisms against themselves, by artificially developing resistance mechanisms in vitro in cancer cell lines and rendering them immunogenic.

Several colorectal human cell lines were selected as suitable candidates for the development of a human colorectal cancer vaccine.

The initial selection of cell lines was made based on their biological and genetic characteristics, so as to cover the largest panel of clinical typologies: microsatellite stability (MSS vs. MSI status); mutated vs. wild-type BRAF, KRAS, PIK3CA, PTEN and TP53 genes; known resistance to common anticancer treatments (fluorouracil [5-FU], oxaliplatin, SN-38 (7-ethyl-10-hydroxy-camptothecin), anti-VEGF antibodies).

Based on the literature and databases (SelTARbase, Cellosaurus and GDSC database), the following cell lines were selected:
LS 174T (ATCC© ref.: CL-188™),
SW620 (ATCC® ref.: CCL-227™),
SW480 (ATCC® ref.: CCL-228™),
LoVo (ATCC® ref.: CCL-229™),
SW48 (ATCC® ref.: CCL-231™),
HCT-116 (ATCC® ref.: CCL-247™), and
HT-29 (ATCC® ref.: HTB-38™)

Pre-Research Cell Bank (RCB): these cell lines were acquired from ATCC and cultured with 10% FBS to obtain seven RCB, but only six out of these seven had an expected growth profile and were viable after a freeze-thaw cycle: SW620 was discarded at this stage (Table 1).

TABLE 1
RCB cultures

| | | Freezing | Viability | Doubling | |
|---|---|---|---|---|---|
| Cell line | Medium | time | at thawing | time | |
| HCT-116 | McCoy's 5A | P3 | 85-92% | 25 | h |
| HT-29 | McCoy's 5A | P3 | 83% | 34 | h |
| LoVo | F12K | P3 | 83% | 45 | h |
| LS 174T | EMEM | P2 | 47-63% | 32.4 | h |
| SW48 | Leibovitz's L15 | P2 | 96% | n.d. | |
| SW480 | Leibovitz's L15 | P3 | 82% | 55 | h |

| | | | | |
|---|---|---|---|---|
| SW620 | Leibovitz's L15 | P3 | n.d. | n.d. |

Pre-RCB 2%: these 6 RCBs, with expected growth profile, have been adapted to low serum culture condition (10% FBS→5% FBS→2% FBS) to mimic the nutriments depletion action observed with anti-VEGF antibodies.

Only four cells lines were able to grow in low serum culture condition and were viable after a freeze-thaw cycle (HCT-116, HT-29, LoVo, and SW480) (Table 2).

TABLE 2
RCB 2% cultures

| | Freezing | Viability | Doubling | |
|---|---|---|---|---|
| Cell line | time | at thawing | time | |
| HCT-116 | P9 | 37-55% | 25-30 | h |
| HT-29 | P6 | 52-78% | 35 | h |
| LoVo | P4 | 28-49% | 40-45 | h |
| LS 174T | P4 | n.d. | 53 | h |
| SW480 | P9 | 93-96% | 60-70 | h |

| | | | |
|---|---|---|---|
| SW48 | P4 | n.d. | n.d. |

These four cell lines were then exposed to two further types of stresses to obtain “DS-A” and “DS-B”.

DS-A: cells were exposed to a physical stress comprising a combination of (1) a low dose ionizing radiation (10 Gy) for 5 minutes, and (2) a thermal stress (1 hour at 42° C.), in order to induce heat shock protein HSP70 expression.

DS-B: cells were exposed to a chemical stress comprising a chemical stimulation with chemotherapeutic agents commonly used for the colorectal cancer: 5-FU (Sigma, F6627), oxaliplatin (Sigma, Y0000271) or SN-38 (7-ethyl-10-hydroxy-camptothecin) (Sigma, H0165). In particular, the association of a chemotherapeutic agent with each cell line was based on literature assumptions (Table 3), and further on the degree of resistance of the cell line to each chemotherapeutic agent. For each cell line, this resistance level to each chemotherapeutic agent was assessed by determining the half maximal inhibitory concentration (IC₅₀) in a cytotoxic assay. IC₅₀ are given in Table 4.

TABLE 3
Chemoresistance of different colorectal human cell lines to
5-FU, oxaliplatin and SN-38, according to the literature.

| Drug | Resistance cell lines |
|---|---|
| 5-FU | HCT-116, HT-29, SW48, SW480 |
| Oxaliplatin | HCT-116, HT-29, LoVo, SW48 |
| SN-38 | HCT-116, HT-29, SW48 |

TABLE 4
IC₅₀ of 5-FU, oxaliplatin and SN-38 in
different colorectal human cell lines.

| | IC₅₀ | | | |
|---|---|---|---|---|
| Cell line | | 5-FU | Oxaliplatin | SN-38 |

| | | | | | |
|---|---|---|---|---|---|
| HT-29 | 3.6 | μM | 460 | nM | 130 nM |
| HCT-116 | 17 | μM | 600 | nM | 50 nM |
| LoVo | 2.4 | μM | 1.1 | μM | 22 nM |

| | | | | |
|---|---|---|---|---|
| SW480 | 23.1 | μM | n.d. | n.d. |

After stress exposure, whether physical or chemical, cells were haptenated, i.e., rendered immunogenic through binding of a carrier molecule—capable of conferring immunogenicity—to the stress proteins expressed as a resistance mechanism in response to the stress, whether these stress proteins be free, bound at the surface or within the interior of the cancer cells. The carrier molecule is dinitrophenyl.

Finally, three cell lines with the best proliferative capacities in culture medium with reduced serum (2% FBS) after stress exposure were selected: LoVo, HCT-116 and HT-29.

Master Cell Bank (MCB): these selected 3 cells lines were cultured in low serum culture condition (2% FBS) to obtain master cell banks (MCB), mimicking a metabolic stress (nutriment depletion observed in treatment therapy with anti-VEGF antibodies).

### Example 2

### Human Colorectal Cancer Vaccine Manufacture Process

Based on the preliminary results obtained in Example 1, we manufactured a vaccine composition comprising multi-stressed, haptenated and non-proliferative HT-29, HCT-116 and LoVo cells.

For each cell line, starting from the MCB, a DS-A and DS-B were manufactured.

For the DS-A, the MCB of each cell line was thawed (if frozen) and cultured in vitro. During the course of their growth phase or their plateau phase, a physical stress comprising a low dose of ionizing radiation (10 Gy) for 5 minutes together with a thermal stress (1 hour at 42° C.) was applied. Stress proteins expressed in reaction to this stress were then haptenated with dinitrophenyl. After formulation at 30 million cells/mL and freezing for storage purposes, these cells (comprising the haptenated stress molecules) were inactived, i.e., rendered non-proliferative, with a high of ionizing radiation (25 Gy), to inhibit cell proliferation while maintaining the cell structure intact.

For the DS-B, the MCB of each cell line was thawed (if frozen) and cultured in vitro. During the course of their growth phase or their plateau phase, a chemical stress comprising a chemical stimulation with chemotherapeutic agents was applied: for HT-29 cells, 13 μM oxaliplatin was applied for 72 hours; for HCT-116 cells, 315 nM SN-38 (7-ethyl-10-hydroxy-camptothecin) was applied for 48 hours (or 100 nM SN-38 in most recent experiments with similar final results); and for LoVo cells, 5 μM 5-FU was applied for 48 hours. Stress proteins expressed in reaction to this stress were then haptenated with dinitrophenyl. After formulation at 30 million cells/mL and freezing for storage purposes, these cells (comprising the haptenated stress molecules) were inactived, i.e., rendered non-proliferative, with a high of ionizing radiation (25 Gy), to inhibit cell proliferation while maintaining the cell structure intact.

A comparison of marker expression was carried out to validate each DS-A and DS-B.

For the three DS-A, the radiation and thermal stress applied to the three cell lines induced the same phenotypic changes in all three DS-A, with an overexpression of HSP70 and CD227 as compared to unstressed cells (Table 5). These data were confirmed with experiments performed on several other cell batches similarly treated, which all confirmed HSP70 and CD227 overexpression after radiation and thermal stress.

TABLE 5
phenotypic changes in HT-29, HCT-116 and LoVo cells, before
and after DS-A treatment, in the RCB (10% FBS, non-treated),
the MCB (2% FBS, non-treated) and the DS-A (2% FBS, treated).
% indicate the percentage of cells expressing the marker
out of the total number of cells. Values in parenthesis
indicate the MFI (mean fluorescence intensity).

| | | | Cmhsp70.1 | CD227 | CD107 |
|---|---|---|---|---|---|
| Cell line | Treatment | | (HSP70) | (MUC1) | (LAMP-1) |
| HT-29 | RCB | 10% FBS | 51% | 14% | 39% |
| | | Non-treated | (50421) | (9985) | (34915) |
| | MCB | 2% FBS | 38% | 18% | 97% |
| | | Non-treated | (131328) | (19461) | (25064) |
| | DS-A | 2% FBS | 90% | 60% | 98% |
| | | 10 Gy + 42° C. | (>350000) | (>13000) | (31617) |
| HCT-116 | RCB | 10% FBS | 76% | 16% | 53% |
| | | Non-treated | (46853) | (11584) | (19160) |
| | MCB | 2% FBS | 70% | 22% | 77% |
| | | Non-treated | (57775) | (15945) | (49348) |
| | DS-A | 2% FBS | 99% | 79% | 55% |
| | | 10 Gy + 42° C. | (252504) | (2241) | (45097) |
| LoVo | RCB | 10% FBS | 75% | 29% | 36% |
| | | Non-treated | (58743) | (13490) | (29510) |
| | MCB | 2% FBS | 83% | 32% | 81% |
| | | Non-treated | (39879) | (9023) | (21491) |
| | DS-A | 2% FBS | 99% | 88% | 85% |
| | | 10 Gy + 42° C. | (200960) | (8246) | (91816) |

For the DS-B, treatment of HT-29 cells with oxaliplatin led to an overexpression of CD95, CD107 and CD54 markers as compared to unstressed cells; and treatment of HCT-116 cells with SN-38 (7-ethyl-10-hydroxy-camptothecin) or LoVo cells with 5-FU led to an overexpression of CD66 marker as compared to unstressed cells, and of CD243 in LoVo treated by 5FU (Table 6). These data were confirmed with experiments performed on several other cell batches similarly treated, which all confirmed:
overexpression of CD66 in HCT-116 cells after treatment with SN-38, regardless of the preliminary cells culture conditions (whether in T25 or T225 CellStacks);
overexpression of CD54, CD95 and CD107 in HT-29 cells after treatment with oxaliplatin; and
overexpression of CD243 (and, to a lesser extent, CD66) in LoVo cells after treatment with 5-FU.

Finally, the vaccine composition (DP or “STC-1010”) could be formulated by pooling all six DS (3 DS-A—one for each cell line; and 3 DS-B—one for each cell line) in 100 μL doses (each comprising 3 million cells). FACS analysis revealed a high expression (assessed as a percentage of cells expressing the marker out of the total number of cells and as mean fluorescence intensity) of HSP70, CD227, CD95 and CD243 in this final product (Table 7).

TABLE 6
phenotypic changes in HT-29, HCT-116 and LoVo cells, before and after DS-B treatment, in the RCB (10% FBS, non-
treated), the MCB (2% FBS, non-treated) and the DS-B (2% FBS, treated). % indicate the percentage of cells expressing
the marker out of the total number of cells. Values in parenthesis indicate the MFI (mean fluorescence intensity).
Two columns for a given marker indicate duplicate results on two different cell batches.

| | | | CD54 | | CD66 | | CD95 | | CD107 | | CD243 |
|---|---|---|---|---|---|---|---|---|---|---|---|
| Cell line | Treatment | | (ICAM-1) | | (CEA) | | (FAS receptor) | | (LAMP-1) | | (MDR-1) |
| HT-29 | RCB | 10% FBS | 35% | 57% | / | / | 8% | 14% | 25% | 30% | / |
| | | Non-treated | (4472) | (3927) | | | (5023) | (7582) | (26420) | (50787) | |
| | MCB | 2% FBS | 47% | 61% | 98% | / | 37% | 58% | 46% | 54% | 2% |
| | | Non-treated | (2910) | (2906) | (19255) | | (5286) | (5277) | (21115) | 20446) | (8310) |
| | DS-B | 2% FBS | 96% | 98% | 98% | / | 86% | 93% | 96% | 73% | 6% |
| | | Oxaliplatin | (15566) | (16010) | (25417) | | (13912) | (13121) | (13689) | (12107) | (10837) |
| HCT-116 | RCB | 10% FBS | / | / | 3% | 2% | / | / | 41% | 53% | / |
| | | Non-treated | | | | | | | (61194) | (19160) | |
| | MCB | 2% FBS | / | | 5% | 5% | / | / | 33% | 28% | 7% |
| | | Non-treated | | | | | | | (45359) | (29726) | (10726) |
| | DS-B | 2% FBS | / | / | 63% | 81% | / | / | 35% | 44% | 4% |
| | | SN-38 | | | (13130) | (22844) | | | (25461) | (20675) | (6693) |
| LoVo | RCB | 10% FBS | / | / | 72% | 72% | 96% | / | 41% | 53% | / |
| | | Non-treated | | | (40510) | (42147) | (21287) | | (52928) | (29510) | |
| | MCB | 2% FBS | 0% | / | 58% | 68% | 68% | / | 33% | 28% | 55% |
| | | Non-treated | | | (88799) | (81155) | (12648) | | (50995) | (38006) | (7711) |
| | DS-B | 2% FBS | 0% | / | 72% | 88% | 75% | / | 35% | 44% | 98% |
| | | 5-FU | | | (127233) | (89793) | (17183) | | (73187) | (46862) | (66072) |

TABLE 7
phenotypic changes in STC-1010 (final product). % indicates the percentage of cells
expressing the marker out of the total number of cells. MFI: mean fluorescence intensity.
Ranges indicate results obtained for 5 different batches (including batches at 3 ×
10⁶ or 3 × 10⁷ cells/mL). CD243 was assessed on a single batch only.

| | | HSP70 | CD227 | CD54 | CD95 | CD66 | CD243 |
|---|---|---|---|---|---|---|---|
| STC-1010 | % | 94-99% | 65-88% | 13-49% | 82-95% | 32-60% | 96% |
| | MFI | 191647-958843 | 15229-19809 | 10145-18830 | 14174-22645 | 21916-49224 | 162377 |

### Example 3

### LC-MS/MS Identification and Relative Quantification of Proteins in the Six Intermediate Compositions

During this study, different cell lines from different taxonomies with and without being subjected to radiations, thermal stress, chemical stress, metabolic stress or combinations thereof in vitro were analyzed.

Expressed proteins were identified in specific databases. An adapted sample preparation was performed to improve protein detection.

Individual amounts of detected proteins were evaluated.

The normalized signal obtains for each protein was compared to the others.

### Material and Methods

During the production, cell lines have to be collected according to gene expression after exposures to different stresses (radiations, thermal stress, chemical stress, metabolic stress or combinations thereof). These different stresses induced an overexpression of antigens. Immunogenicity has also been enhanced by chemically marking the surface proteins with haptens. Protein expression was compared between different cell samples after exposure to different stresses. A summary of the different compositions analyzed in this study is given in Table 8.

TABLE 8

| Sample | | Viability | |
|---|---|---|---|
| identification | Cell concentration | (Yes/No) | Medium |
| HT-29 RCB | 1 mL ≈ 6.10⁶ cells | Yes | McCoy's 5A, 2% FBS, 10% DMSO |
| HCT-116 RCB | 1 mL ≈ 6.10⁶ cells | Yes | McCoy's 5A, 2% FBS, 10% DMSO |
| LoVo RCB | 1 mL ≈ 6.10⁶ cells | Yes | F12K, 2% FBS, 10% DMSO |
| HT-29 MCB | 1 mL ≈> 6.10⁶ cells | Yes | McCoy's 5A, 2% FBS, 10% DMSO |
| HCT-116 MCB | 1 mL ≈> 6.10⁶ cells | Yes | McCoy's 5A, 2% FBS, 10% DMSO |
| LoVo MCB | 1 mL ≈> 6.10⁶ cells | Yes | F12K, 2% FBS, 10% DMSO |
| LoVo DS-A | 0.5 mL ≈ 5.10⁵ cells | No | Earle's Balanced Salt Solution |
| | | | (EBSS), saccharose, 5% DMSO |
| LoVo DS-B | 0.5 mL ≈ 5.10⁵ cells | No | EBSS, saccharose, 5% DMSO |
| HT-29 DS-A | 0.5 mL ≈ 5.10⁵ cells | No | EBSS, saccharose, 5% DMSO |
| HT-29 DS-B | 0.5 mL ≈ 5.10⁵ cells | No | EBSS, saccharose, 5% DMSO |
| HCT-116 DS-A | 0.5 mL ≈ 5.10⁵ cells | No | EBSS, saccharose, 5% DMSO |
| HCT-116 DS-B | 0.5 mL ≈ 5.10⁵ cells | No | EBSS, saccharose, 5% DMSO |
| DP | 0.75 mL ≈ 3.10⁶ cells | No | EBSS, saccharose, 5% DMSO |

### Sample Preparation Process

The sample preparation process applied to cells pellets in order to detect proteins comprised a chemical lysis of the cells followed by protein digestion. Each generated peptide was separated according to physicochemical property using a nanoflow chromatographic system coupled to a high-resolution mass spectrometer (NanoLC-MS/MS). The main advantage of using this technology is to improve the sensitivity of the instrument and increase the number of identified proteins.

### Analytical Method

NanoLC-MS/MS analysis were performed in Data Dependent Analysis (DDA) mode, also called shotgun proteomics or peptide mapping. This analytical tool allows acquiring MS and MS/MS spectrum of thousands of peptides through the whole chromatographic separation.

### Protein Identification

Once the experimental MS and MS/MS spectrum acquired, data process was performed using a software search engine that uses mass spectrometry data to identify proteins from proteome databases. In this case, experimental data was correlated to the full human proteome (SWISS Prot databases) for proteins identification.

To validate the identified proteins, some identification parameters were implemented to ensure the specificity and eliminate false positives. Protein identification was performed with 2 peptides with at least one protein-specific peptide in order to be very stringent about the identified proteins. The identified proteins were the ones present in higher amount. Moreover, for the identification process, some frequently observed peptide modifications such as methionine oxidation or pyroglutamic acid formation from glutamine were added to the search engine.

A protein amount normalization was also done.

### Individual Quantification Evaluation

This strategy comprised spiking a universal calibration curve of synthetic peptides accurately calibrated thanks to the Readybeads™ technology (Anaquant, Villeurbanne, France). This calibration curve allowed the conversion of peptide signal in quantity of all identified proteins. Peptides used in the calibration curve have been chosen for their specificity; their sequences do not correspond to any of the proteome of the cell lines used in bioprocess.

Those standards represent an accurate calibration curve adapted to quantitate an amount of any proteins. The reproducibility and the stability of this calibration curve are ensured thanks to the Readybeads™ technology. All identified peptides and proteins can be normalized using this calibration curve. The normalized signal allowed batch-to-batch comparison.

### Results

The 13 samples were treated and analyzed in parallel. Some pellet samples were difficult to lyse, and in order to compare same injected protein quantity, protein quantitation was performed using the Pierce™ BCA Protein Assay Kit before digestion. Despite this quantification step, after sample analysis, injected protein quantity seemed highly different between samples.

This difference could either come from a difference in protein quantity injected, or from a difference in protein dynamic range in the samples.

In order to evaluate those hypotheses, AQTBeads added to sample were used as quality control. Readybeads™ quality control showed a good linearity with slope and r²>0.9 and quantification through the range 1 to 500 fmol injected protein quantity. Peptide digests were also quantified after LC-MS injection. Results are reported in Table 9.

TABLE 9
quality control parameters.

| | | | | | | Number of | Number of | Peptide |
|---|---|---|---|---|---|---|---|---|
| Sample | | | | QC No1 | QC No2 | identified | quantified | quantification |
| identification | R² | Slope | Intercept | (10 fmol) | (5 fmol) | proteins | proteins | (μg/mL) |
| HT-29 RCB | 0.96 | 1 | 22.2 | 7 | 5 | 1841 | 745 | 420 |
| HCT-116 RCB | 0.97 | 0.97 | 22. | 6 | 5 | 1667 | 491 | <LOD |
| LoVo RCB | 0.97 | 0.98 | 22.5 | 9 | 5 | 1652 | 555 | 376 |
| HT-29 MCB | 0.96 | 0.99 | 22.3 | 9 | 6 | 1881 | 790 | 475 |
| HCT-116 MCB | 0.96 | 0.98 | 22.4 | 5 | 5 | 1856 | 646 | 342 |
| LoVo MCB | 0.95 | 0.95 | 22.6 | 6 | 4 | 1466 | 394 | 698 |
| LoVo DS-A | 0.96 | 1 | 22.1 | 11 | 6 | 2002 | 1151 | NA |
| LoVo DS-B | 0.95 | 0.94 | 22.6 | 15 | 4 | 1102 | 105 | 498 |
| HT-29 DS-A | 0.95 | 0.94 | 22.7 | 12 | 4 | 1179 | 98 | 498 |
| HT-29 DS-B | 0.94 | 0.93 | 22.8 | 12 | 4 | 563 | 21 | 265 |
| HCT-116 DS-A | 0.95 | 0.99 | 22.1 | 10 | 6 | 2118 | 1369 | 787 |
| HCT-116 DS-B | 0.96 | 0.96 | 22.5 | 16 | 5 | 1341 | 208 | 465 |
| DP | 0.96 | 0.96 | 22.6 | 9 | 5 | 1556 | 333 | 365 |

### Global Protein Comparison

Untreated Cells

RCB (Research Cell Bank) samples were untreated samples in 10% FBS medium.

MCB (Master Cell Bank) samples come from RCB with a medium adaptation (2% FBS).

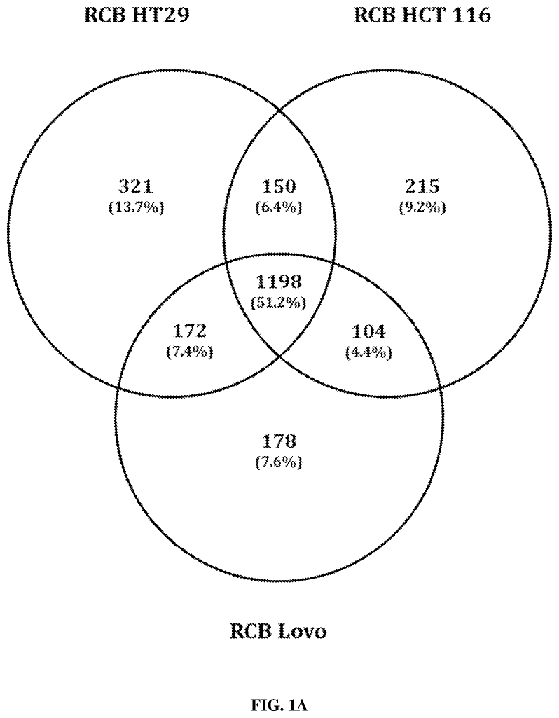

The proteins identified in the three human cell lines (HT-29, HCT-116 and LoVo) were compared (FIGS. 1A and 1B).

Almost 50% of identified proteins were commonly identified in all three, untreated, human cell lines. This result was expected since these three cell lines are derived from colon or colorectal carcinomas.

DS-A Treatment

DS-A treatment comprises exposure of the cells to a low dose of ionizing radiation (10 Gy) for 5 minutes together with a thermal stress (1 hour at 42° C.). These cells were also subjected to a metabolic stress (i.e., a medium adaptation from 10% to 2% FBS between RCB and MCB).

After this DS-A treatment, some proteins appeared to be over-expressed in all three cell lines (protein proportion ratio between DS-A samples and MCB sample >2.5 with respect to global protein quantity). These proteins are reported in Table 10.

TABLE 10
overexpressed proteins after DS-A treatment. The ratio is between protein
proportion (with respect to global protein quantity) measured in DS-A
versus MCB samples. Highlighted in bold are proteins specifically
identified only in the DS-A samples but not in the MCB samples.

| Uniprot | | HT-29 DS-A | HCT-116 DS-A | LoVo DS-A |
|---|---|---|---|---|
| accession | | vs. | vs. | vs. |
| number | Protein | HT-29 MCB | HCT-116 MCB | LoVo MCB |
| Q15366 | Poly(rC)-binding | 590.0 | 908.0 | 1111.6 |
| | protein 2 | | | |
| Q92888 | Rho guanine | 590.0 | 34.9 | 148.2 |
| | nucleotide | | | |
| | exchange factor 1 | | | |
| Q14789 | Golgin subfamily B | 590.0 | 331.8 | 129.7 |
| | member 1 | | | |
| P02749 | Beta-2-glycoprotein | 295.0 | 52.4 | 240.8 |
| | 1 | | | |
| Q9Y446 | Plakophilin-3 | 295.0 | 279.4 | 37.1 |
| Q6ZRV2 | Protein FAM83H | 295.0 | 139.7 | 55.6 |
| O15027 | Protein transport | 295.0 | 87.3 | 259.4 |
| | protein Sec16A | | | |
| Q27J81 | Inverted formin-2 | 295.0 | 52.4 | 111.2 |
| Q9NQW6 | Anillin | 295.0 | 192.1 | 222.3 |
| Q9H8V3 | Protein ECT2 | 295.0 | 34.9 | 74.1 |
| Q15149 | Plectin | 9.7 | 3.6 | 22.5 |
| P58107 | Epiplakin | 7.7 | 3.9 | 1908.2 |
| P46013 | Proliferation marker | 5.8 | 3.3 | 3.2 |
| | protein Ki-67 | | | |
| Q12907 | Vesicular | 5.5 | 261.9 | 7.5 |
| | integral-membrane | | | |
| | protein VIP36 | | | |
| P11279 | Lysosome-associated | 2.5 | 8.9 | 74.1 |
| | membrane | | | |
| | glycoprotein 1 | | | |

These ten proteins which are identified only in the DS-A samples but not in the MCB samples reflect that the DS-A treatment led the cells to develop a resistance mechanism by which they have produced stress proteins.

DS-B Treatment

DS-B treatment comprises exposure of the cells to chemotherapeutic agents, namely 13 μM oxaliplatin applied for 72 hours on HT-29 cells; 315 nM SN-38 (7-ethyl-10-hydroxy-camptothecin) applied for 48 hours on HCT-116 cells; and 5 μM 5-FU applied for 48 hours on LoVo cells. These cells were also subjected to a metabolic stress (i.e., a medium adaptation from 10% to 2% FBS between RCB and MCB).

After this DS-B treatment, some proteins appeared to be over-expressed (protein proportion ratio between DS-B samples and MCB sample >2.5 with respect to global protein quantity). These proteins are reported in Table 11.

TABLE 11
overexpressed proteins after DS-B treatment. The ratio is between protein
proportion (with respect to global protein quantity) measured in DS-B
versus MCB samples. Highlighted in bold are proteins specifically
identified only in the DS-B samples but not in the MCB samples.

| Uniprot | | HT-29 DS-B | HCT-116 DS-B | LoVo DS-B |
|---|---|---|---|---|
| accession | | vs. | vs. | vs. |
| number | Protein | HT-29 MCB | HCT-116 MCB | LoVo MCB |
| P02749 | Beta-2- | 10.4 | 342.0 | 343.8 |
| | glycoprotein 1 | | | |
| P0C0S8 | Histone H2A type 1 | 16674.8 | 11284.4 | 21313.9 |
| Q15366 | Poly(rC)-binding | 10.4 | 854.9 | 687.5 |
| | protein 2 | | | |
| Q86UP2 | Kinectin | 10.4 | 2.5 | 4.4 |
| Q15149 | Plectin | 35.0 | 19.7 | 5.0 |
| O95182 | NADH dehydrogenase | 10.4 | 3.3 | 3.5 |
| | [ubiquinone] 1 alpha | | | |
| | subcomplex subunit 7 | | | |

Overexpressed proteins seemed mainly associated to DNA reparation. In order to go deeper in the results analysis, we decided to focus on membrane proteins.

### Focus on Membrane Protein

Untreated Cells

Tables 12 to 14 represent the membrane proteins overexpressed (protein proportion ratio >2.5 with respect to global protein quantity) in one human cell line (untreated RCB and MCB) compared with the two others.

TABLE 12
membrane proteins overexpressed in HT-29 RCB and MCB
compared with HCT-116 and LoVo RCB and MCB samples.

| Uniprot accession | |
|---|---|
| number | Protein |
| Q9NQC3 | Reticulon-4 |
| P04844 | Dolichyl-diphosphooligosaccharide--protein |
| | glycosyltransferase subunit 2 |
| P23229 | Integrin alpha-6 |
| P51572 | B-cell receptor-associated protein 31 |
| O95573 | Long-chain-fatty-acid--CoA ligase 3 |
| Q15758 | Neutral amino acid transporter B(0) |
| P53007 | Tricarboxylate transport protein, |
| | mitochondrial |
| O60488 | Long-chain-fatty-acid--CoA ligase 4 |
| O75746 | Calcium-binding mitochondrial carrier |
| | protein Aralar1 |
| Q9Y6C9 | Mitochondrial carrier homolog 2 |
| Q9HDC9 | Adipocyte plasma membrane-associated protein |
| P07099 | Epoxide hydrolase 1 |
| Q13724 | Mannosyl-oligosaccharide glucosidase |
| O00264 | Membrane-associated progesterone receptor |
| | component 1 |
| A0FGR8 | Extended synaptotagmin-2 |
| Q15738 | Sterol-4-alpha-carboxylate 3-dehydrogenase, |
| | decarboxylating |
| Q9Y5M8 | Signal recognition particle receptor |
| | subunit beta |
| O15270 | Serine palmitoyltransferase 2 |
| Q8WY22 | BRI3-binding protein |
| O43676 | NADH dehydrogenase [ubiquinone] 1 beta |
| | subcomplex subunit 3 |
| Q9Y3D6 | Mitochondrial fission 1 protein |

TABLE 13
membrane proteins overexpressed in HCT-116 RCB and MCB
compared with HT-29 and LoVo RCB and MCB samples.

| Uniprot accession number | Protein |
|---|---|
| Q96AG4 | Leucine-rich repeat-containing protein 59 |
| O43169 | Cytochrome b5 type B |

TABLE 14
membrane proteins overexpressed in LoVo RCB and MCB
compared with HT-29 and HCT-116 RCB and MCB samples.

| Uniprot accession number | Protein |
|---|---|
| Q13423 | NAD(P) transhydrogenase, mitochondrial |

DS-A Treatment

DS-A treatment comprises exposure of the cells to a low dose of ionizing radiation (10 Gy) for 5 minutes together with a thermal stress (1 hour at 42° C.). These cells were also subjected to a metabolic stress (i.e., a medium adaptation from 10% to 2% FBS between RCB and MCB).

Membrane proteins appeared to be mainly overexpressed rather than underexpressed.

Tables 15 to 17 describe the membrane proteins overexpressed (protein proportion ratio >2.5 with respect to global protein quantity) in each human cell line (DS-A sample) compared to its corresponding MCB sample.

TABLE 15
membrane proteins overexpressed in
HT-29 DS-A compared with HT-29 MCB.

| Uniprot accession number | Protein |
|---|---|
| Q12907 | Vesicular integral-membrane protein |
| | VIP36 |
| P11279 | Lysosome-associated membrane |
| | glycoprotein 1 |
| Q9P2E9 | Ribosome-binding protein 1 |
| O43291 | Kunitz-type protease inhibitor 2 |
| Q6P1A2 | Lysophospholipid acyltransferase 5 |
| P50402 | Emerin |
| Q86UE4 | Protein LYRIC |
| Q9BW60 | Elongation of very long chain fatty |
| | acids protein 1 |
| P60059 | Protein transport protein Sec61 subunit |
| | gamma |

TABLE 16
membrane proteins overexpressed in HCT-
116 DS-A compared with HCT-116 MCB.

| Uniprot accession | |
|---|---|
| number | Protein |
| P23229 | Integrin alpha-6 |
| P11279 | Lysosome-associated membrane glycoprotein 1 |
| Q9P2E9 | Ribosome-binding protein 1 |
| Q14739 | Delta(14)-sterol reductase LBR |
| O95202 | Mitochondrial proton/calcium exchanger protein |
| Q5JTH9 | RRP12-like protein |
| Q9BSJ8 | Extended syptotagmin-1 |
| P61619 | Protein transport protein Sec61 subunit alpha |
| | isoform 1 |
| P11166 | Solute carrier family 2, facilitated glucose |
| | transporter member 1 |
| Q14126 | Desmoglein-2 |
| Q86UP2 | Kinectin |
| Q86UE4 | Protein LYRIC |
| O95470 | Sphingosine-1-phosphate lyase 1 |
| Q9P0L0 | Vesicle-associated membrane protein-associated |
| | protein A |
| Q96N66 | Lysophospholipid acyltransferase 7 |
| Q9Y5M8 | Sigl recognition particle receptor subunit beta |
| Q5JTV8 | Torsin-1A-interacting protein 1 |
| Q8N766 | ER membrane protein complex subunit 1 |
| Q15738 | Sterol-4-alpha-carboxylate 3-dehydrogese, |
| | decarboxylating |
| O00264 | Membrane-associated progesterone receptor |
| | component 1 |
| P16070 | CD44 antigen |
| Q9BUR5 | MICOS complex subunit MIC26 |
| Q7Z7H5 | Transmembrane emp24 domain-containing protein 4 |
| Q53FV1 | ORM1-like protein 2 |
| Q9P2X0 | Dolichol-phosphate mannosyltransferase subunit 3 |
| Q07065 | Cytoskeleton-associated protein 4 |
| Q9BTV4 | Transmembrane protein 43 |
| Q8TC12 | Retinol dehydrogese 11 |
| Q15005 | Sigl peptidase complex subunit 2 |
| O15270 | Serine palmitoyltransferase 2 |
| Q96A33 | Coiled-coil domain-containing protein 47 |
| Q9NZ01 | Very-long-chain enoyl-CoA reductase |
| Q14517 | Protocadherin Fat 1 |
| Q9Y3B3 | Transmembrane emp24 domain-containing protein 7 |
| Q03135 | Caveolin-1 |
| Q6ZNB6 | NF-X1-type zinc finger protein NFXL1 |
| Q96HR9 | Receptor expression-enhancing protein 6 |
| Q9BW72 | HIG1 domain family member 2A, mitochondrial |
| Q8N5G0 | Small integral membrane protein 20 |
| Q9P0J0 | DH dehydrogese [ubiquinone] 1 alpha subcomplex |
| | subunit 13 |
| O43676 | DH dehydrogese [ubiquinone] 1 beta subcomplex |
| | subunit 3 |
| Q9HC21 | Mitochondrial thiamine pyrophosphate carrier |
| Q9Y3D6 | Mitochondrial fission 1 protein |
| P27338 | Amine oxidase [flavin-containing] B |
| Q15070 | Mitochondrial inner membrane protein OXA1L |
| A1L0T0 | 2-hydroxyacyl-CoA lyase 2 |
| Q969V3 | Nicalin |
| P20020 | Plasma membrane calcium-transporting ATPase 1 |
| Q9NZM1 | Myoferlin |
| P11717 | Cation-independent mannose-6-phosphate receptor |

TABLE 17
membrane proteins overexpressed in
LoVo DS-A compared with LoVo MCB.

| Uniprot accession | |
|---|---|
| number | Protein |
| Q12907 | Vesicular integral-membrane protein VIP36 |
| Q9BVI4 | Nucleolar complex protein 4 homolog |
| P67812 | Sigl peptidase complex catalytic subunit SEC11A |
| Q9Y5M8 | Sigl recognition particle receptor subunit beta |
| P10620 | Microsomal glutathione S-transferase 1 |
| P16435 | DPH--cytochrome P450 reductase |
| Q9P035 | Very-long-chain (3R)-3-hydroxyacyl-CoA |
| | dehydratase 3 |
| Q13724 | Mannosyl-oligosaccharide glucosidase |
| Q9BZF1 | Oxysterol-binding protein-related protein 8 |
| Q00765 | Receptor expression-enhancing protein 5 |
| Q9BUR5 | MICOS complex subunit MIC26 |
| O95395 | Beta-1,3-galactosyl-O-glycosyl-glycoprotein |
| | beta-1,6-N-acetylglucosaminyltransferase 3 |
| Q96IX5 | ATP synthase membrane subunit DAPIT, |
| | mitochondrial |
| P56134 | ATP synthase subunit f, mitochondrial |
| Q8N5K1 | CDGSH iron-sulfur domain-containing protein 2 |
| Q8N0U8 | Vitamin K epoxide reductase complex subunit 1-like |
| | protein 1 |
| Q96A26 | Protein FAM162A |
| O75477 | Erlin-1 |
| P33121 | Long-chain-fatty-acid--CoA ligase 1 |
| O43169 | CYB5B_HUMAN Cytochrome b5 type B |
| Q8TC12 | Retinol dehydrogese 11 |
| Q15363 | Transmembrane emp24 domain-containing protein 2 |
| Q9NZ01 | Very-long-chain enoyl-CoA reductase |
| Q15125 | 3-beta-hydroxysteroid-Delta(8),Delta(7)-isomerase |
| P53985 | Monocarboxylate transporter 1 |
| P01889 | HLA class I histocompatibility antigen, B |
| | alpha chain |
| O94905 | Erlin-2 |
| P21926 | CD9 antigen |
| P03928 | ATP synthase protein 8 |
| Q9UDX5 | Mitochondrial fission process protein 1 |
| O00483 | Cytochrome c oxidase subunit NDUFA4 |
| Q9P0J0 | DH dehydrogese [ubiquinone] 1 alpha |
| | subcomplex subunit 13 |
| O95168 | DH dehydrogese [ubiquinone] 1 beta subcomplex |
| | subunit 4 |
| O43676 | DH dehydrogese [ubiquinone] 1 beta subcomplex |
| | subunit 3 |
| Q9Y3D6 | Mitochondrial fission 1 protein |
| Q8WY22 | BRI3-binding protein |

DS-B Treatment

DS-B treatment comprises exposure of the cells to chemotherapeutic agents, namely 13 μM oxaliplatin applied for 72 hours on HT-29 cells; 315 nM SN-38 (7-ethyl-10-hydroxy-camptothecin) applied for 48 hours on HCT-116 cells; and 5 μM 5-FU applied for 48 hours on LoVo cells. These cells were also subjected to a metabolic stress (i.e., a medium adaptation from 10% to 2% FBS between RCB and MCB).

Membrane proteins appeared to be mainly overexpressed rather than underexpressed.

Tables 18 to 20 describe the membrane proteins overexpressed (protein proportion ratio >2.5 with respect to global protein quantity) in each human cell line (DS-B sample) compared to its corresponding MCB sample.

TABLE 18
membrane proteins overexpressed in
HT-29 DS-B compared with HT-29 MCB.

| Uniprot accession number | Protein |
|---|---|
| Q9P2E9 | Ribosome-binding protein 1 |
| P16435 | NADPH--cytochrome P450 reductase |

TABLE 19
membrane proteins overexpressed in HCT-
116 DS-B compared with HCT-116 MCB.

| Uniprot accession | |
|---|---|
| number | Protein |
| Q00325 | Phosphate carrier protein, mitochondrial |
| P16144 | Integrin beta-4 |
| P11279 | Lysosome-associated membrane glycoprotein 1 |
| P16070 | CD44 antigen |
| Q9P2E9 | Ribosome-binding protein 1 |
| P61619 | Protein transport protein Sec61 subunit alpha |
| | isoform 1 |
| Q86UP2 | Kinectin |
| P04439 | HLA class I histocompatibility antigen, A alpha chain |
| Q96N66 | Lysophospholipid acyltransferase 7 |
| O00264 | Membrane-associated progesterone receptor |
| | component 1 |
| P10620 | Microsomal glutathione S-transferase 1 |
| O95168 | NADH dehydrogenase [ubiquinone] 1 beta |
| | subcomplex subunit 4 |
| Q14126 | Desmoglein-2 |
| P26006 | Integrin alpha-3 |
| Q5JTV8 | Torsin-1A-interacting protein 1 |
| P20020 | Plasma membrane calcium-transporting ATPase 1 |
| O95470 | Sphingosine-1-phosphate lyase 1 |
| Q93050 | V-type proton ATPase 116 kDa subunit a1 |
| Q15070 | Mitochondrial inner membrane protein OXA1L |
| O43674 | NADH dehydrogenase [ubiquinone] 1 beta |
| | subcomplex subunit 5, mitochondrial |
| Q9BTV4 | Transmembrane protein 43 |
| P27338 | Amine oxidase [flavin-containing] B |
| P60468 | Protein transport protein Sec61 subunit beta |
| O14828 | Secretory carrier-associated membrane protein 3 |
| Q96A26 | Protein FAM162A |
| Q8TC12 | Retinol dehydrogenase 11 |
| Q9NVJ2 | ADP-ribosylation factor-like protein 8B |
| O43676 | NADH dehydrogenase [ubiquinone] 1 beta |
| | subcomplex subunit 3 |
| Q9P2X0 | Dolichol-phosphate mannosyltransferase subunit 3 |
| Q9HC21 | Mitochondrial thiamine pyrophosphate carrier |
| Q53FV1 | ORM1-like protein 2 |

TABLE 20
membrane proteins overexpressed in
LoVo DS-B compared with LoVo MCB.

| Uniprot accession | |
|---|---|
| number | Protein |
| Q8TEM1 | Nuclear pore membrane glycoprotein 210 |
| Q9NQC3 | Reticulon-4 |
| P16435 | NADPH--cytochrome P450 reductase |
| O95573 | Long-chain-fatty-acid--CoA ligase 3 |
| O60488 | Long-chain-fatty-acid--CoA ligase 4 |
| Q9P0L0 | Vesicle-associated membrane protein-associated |
| | protein A |
| P53007 | Tricarboxylate transport protein, mitochondrial |
| Q86UP2 | Kinectin |
| O95292 | Vesicle-associated membrane protein-associated |
| | protein B/C |
| Q9P035 | Very-long-chain (3R)-3-hydroxyacyl-CoA |
| | dehydratase 3 |
| Q12907 | Vesicular integral-membrane protein VIP36 |
| Q9NZ01 | Very-long-chain enoyl-CoA reductase |
| P10620 | Microsomal glutathione S-transferase 1 |
| O43169 | Cytochrome b5 type B |
| Q96IX5 | ATP synthase membrane subunit DAPIT, |
| | mitochondrial |
| P56134 | ATP synthase subunit f, mitochondrial |
| P00403 | Cytochrome c oxidase subunit 2 |
| P08574 | Cytochrome c1, heme protein, mitochondrial |
| P01889 | HLA class I histocompatibility antigen, B alpha |
| | chain |
| Q86UE4 | Protein LYRIC |
| P53985 | Monocarboxylate transporter 1 |
| O00483 | Cytochrome c oxidase subunit NDUFA4 |
| Q13724 | Mannosyl-oligosaccharide glucosidase |
| P67812 | Signal peptidase complex catalytic subunit SEC11A |
| Q96A26 | Protein FAM162A |
| Q15363 | Transmembrane emp24 domain-containing protein 2 |
| Q8TC12 | Retinol dehydrogenase 11 |
| O75477 | Erlin-1 |
| Q8WY22 | BRI3-binding protein |
| Q15125 | 3-beta-hydroxysteroid-Delta(8),Delta(7)-isomerase |
| Q00765 | Receptor expression-enhancing protein 5 |
| Q8N5K1 | CDGSH iron-sulfur domain-containing protein 2 |
| P21926 | CD9 antigen |
| A0FGR8 | Extended synaptotagmin-2 |
| O14975 | Very long-chain acyl-CoA synthetase |
| O43291 | Kunitz-type protease inhibitor 2 |
| Q13740 | CD166 antigen |
| P33121 | Long-chain-fatty-acid--CoA ligase 1 |
| Q08722 | Leukocyte surface antigen CD47 |
| O15533 | Tapasin |
| O95395 | Beta-1,3-galactosyl-O-glycosyl-glycoprotein |
| | beta-1,6-N-acetylglucosaminyltransferase 3 |

### Example 4

### LC-MS/MS Identification and Relative Quantification of Proteins in the Final Composition

This study aimed at identifying differentially expressed protein in the final vaccine composition (DP, comprising all six DS described above, and pooled together: 3 DS-A—one for each cell line; and 3 DS-B—one for each cell line).

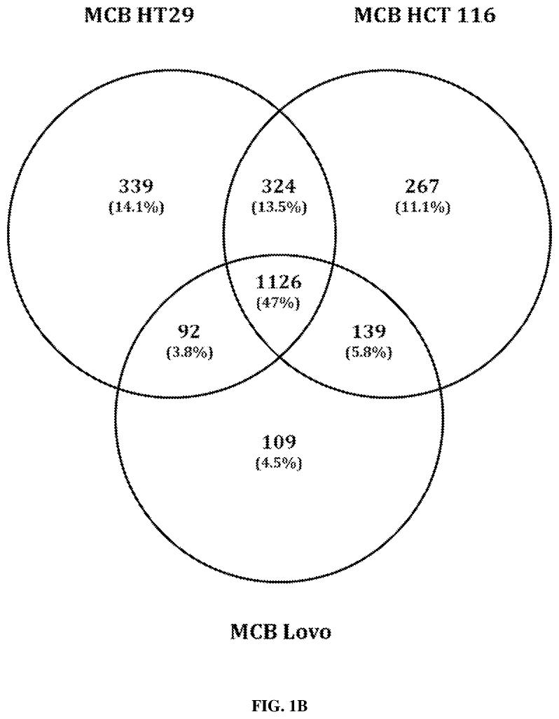

FIG. 2 shows the differential number of proteins expressed in the HCT-116 MCB, DS-A, DS-B and in the final product (DP) comprising all six DS described above (hence including the HCT-116 DS-A and DS-B), by comparison to the initial HCT-116 RCB cultured in classical conditions (with 10% FBS). “+” indicates proteins that are newly expressed, “−” indicates the number of proteins which are no longer expressed at all.

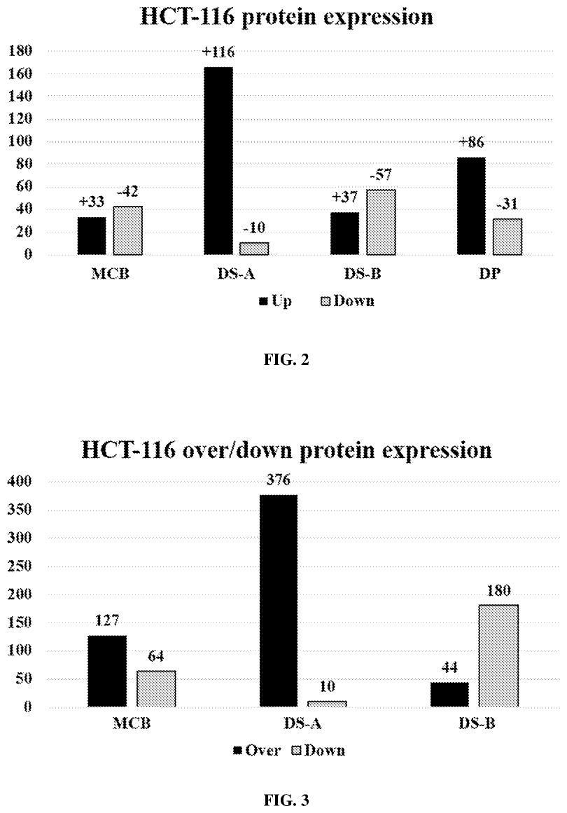

FIG. 3 shows the number of proteins over- and under-expressed in the HCT-116 MCB, DS-A and DS-B, by comparison to the initial HCT-116 RCB cultured in classical conditions (with 10% FBS).

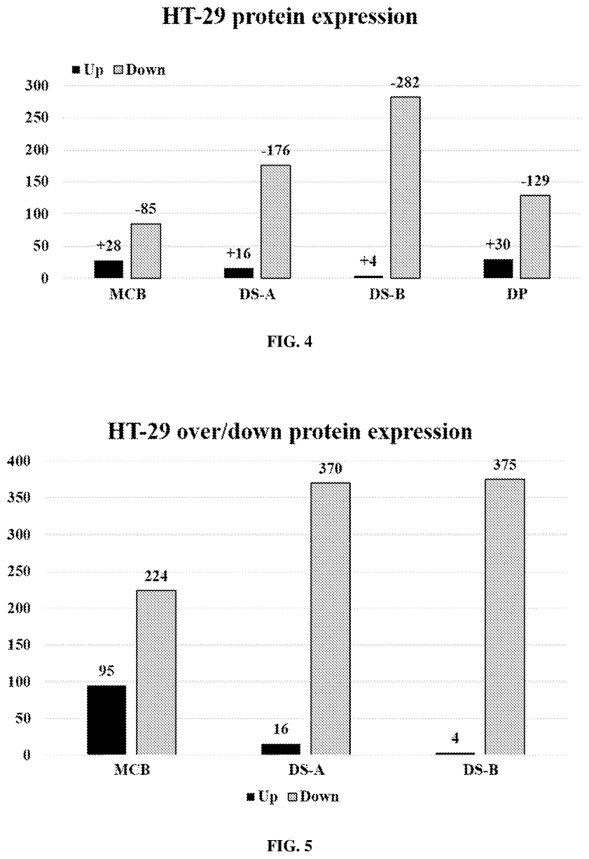

FIG. 4 shows the differential number of proteins expressed in the HT-29 MCB, DS-A, DS-B and in the final product (DP) comprising all six DS described above (hence including the HT-29 DS-A and DS-B), by comparison to the initial HT-29 RCB cultured in classical conditions (with 10% FBS). “+” indicates proteins that are newly expressed, “−” indicates the number of proteins which are no longer expressed at all.

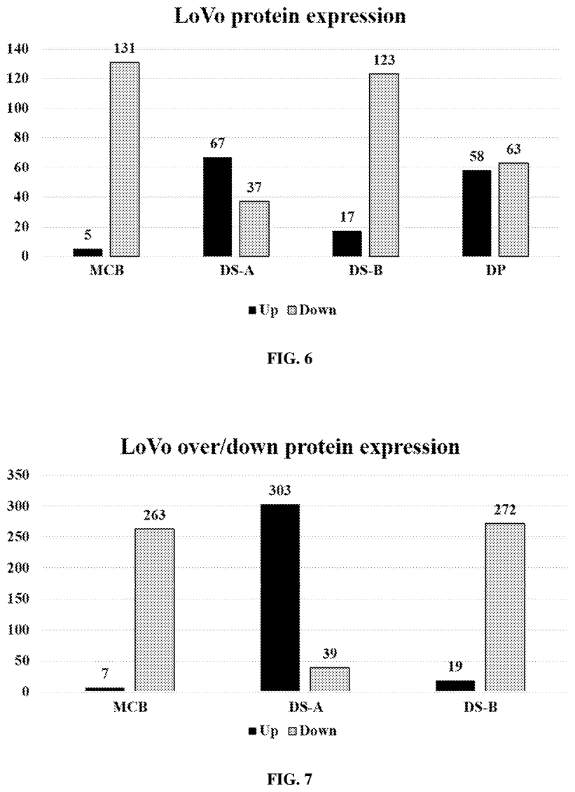

FIG. 5 shows the number of proteins over- and under-expressed in the HT-29 MCB, DS-A and DS-B, by comparison to the initial HT-29 RCB cultured in classical conditions (with 10% FBS).

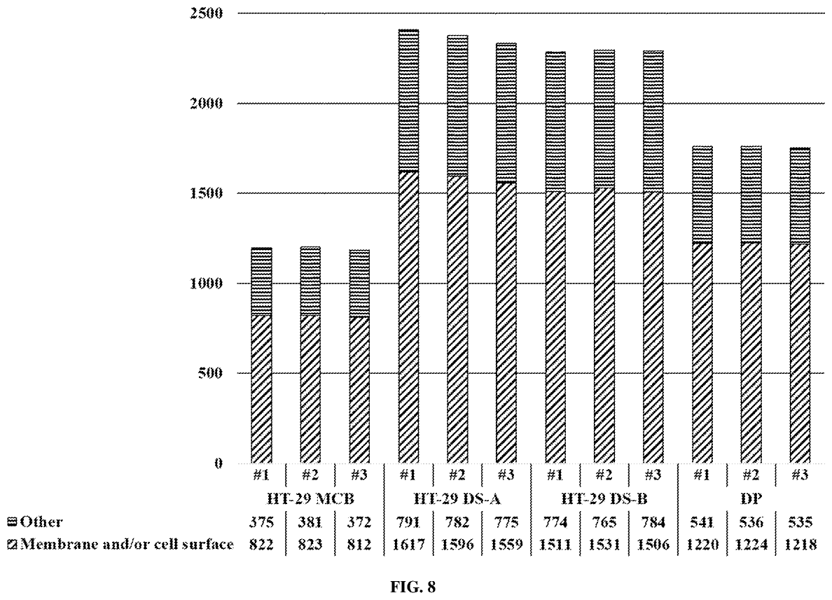

FIG. 6 shows the differential number of proteins expressed in the LoVo MCB, DS-A, DS-B and in the final product (DP) comprising all six DS described above (hence including the LoVo DS-A and DS-B), by comparison to the initial LoVo RCB cultured in classical conditions (with 10% FBS). “+” indicates proteins that are newly expressed, “−” indicates the number of proteins which are no longer expressed at all.

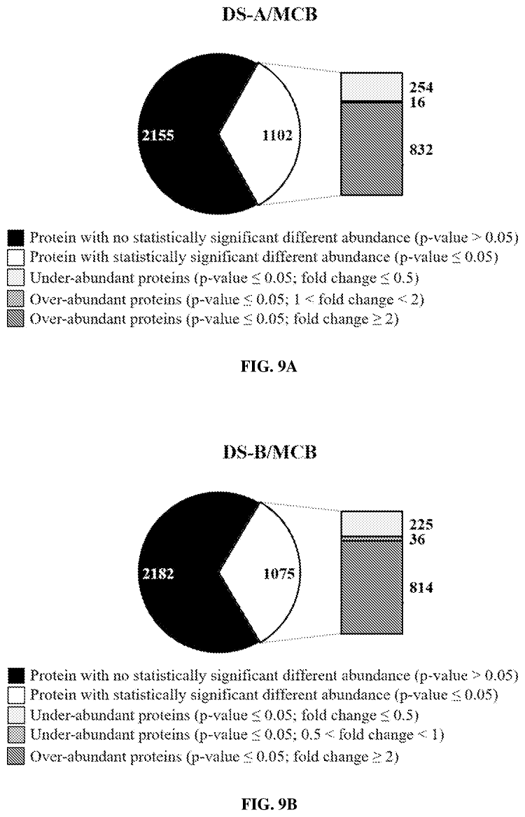

FIG. 7 shows the number of proteins over- and under-expressed in the LoVo MCB, DS-A and DS-B, by comparison to the initial LoVo RCB cultured in classical conditions (with 10% FBS).

A comparison of these LC/MS data is given in Table 21, indicating the number and percentage of proteins which are exclusively present in the final composition (DP) (i.e., not identified in any of the three cell lines at RCB stage), which are over-expressed in the final composition (DP) (in comparison to the three cell lines at RCB stage), and which are similarly or less expressed in the final composition (DP) (in comparison to the three cell lines at RCB stage).

TABLE 21
comparison of the relative expression of proteins identified
in the final composition (DP), in comparison to the three cell
lines at RCB stage. Proteins identified in each RCB but not
identified in the final composition (DP) are not counted. Comparison
was done by applying a multiplication factor 3 for the final
composition (DP), to take the dilution factor 3 due to the mixing
of the 3 cell lines in this DP into account.

| Differential expression | | |
|---|---|---|
| DP versus RCBs | Number of proteins | % |
| Similarly or less expressed in the DP | 1419 | 91% |
| Over-expressed in the DP | 49 | 3% |
| Exclusively present in the DP | 88 | 6% |
| | 1556 | 100% |

Table 22 shows the list of proteins which are exclusively found in the final composition (DP) but not in any of the RCBs. The presence of any of these proteins is therefore characteristic of the final composition having undergone all the different treatments described above.

TABLE 22
proteins exclusively found in the final composition (DP).

| Uniprot accession | |
|---|---|
| number | Protein |
| P08631 | Tyrosine-protein kinase HCK |
| O95758 | Polypyrimidine tract-binding protein 3 |
| Q63HN8 | E3 ubiquitin-protein ligase RNF213 |
| Q9BRL6 | Serine/arginine-rich splicing factor 8 |
| Q9H223 | EH domain-containing protein 4 |
| Q8WWI1 | LIM domain only protein 7 |
| Q9H9Y6 | DNA-directed RNA polymerase I subunit RPA2 |
| Q9Y6K5 | 2′-5′-oligoadenylate synthase 3 |
| O75717 | WD repeat and HMG-box DNA-binding protein 1 |
| P02749 | Beta-2-glycoprotein 1 |
| P36873 | Serine/threonine-protein phosphatase PP1-gamma |
| | catalytic subunit |
| Q9NQW6 | Anillin |
| O43795 | Unconventional myosin-Ib |
| O94973 | AP-2 complex subunit alpha-2 |
| P24941 | Cyclin-dependent kinase 2 |
| P42224 | Signal transducer and activator of transcription |
| | 1-alpha/beta |
| Q14671 | Pumilio homolog 1 |
| Q8NE71 | ATP-binding cassette sub-family F member 1 |
| Q9H0H5 | Rac GTPase-activating protein 1 |
| Q9P2M7 | Cingulin |
| O00186 | Syntaxin-binding protein 3 |
| O43772 | Mitochondrial carnitine/acylcarnitine carrier protein |
| O60684 | Importin subunit alpha-7 |
| O75676 | Ribosomal protein S6 kinase alpha-4 |
| P20339 | Ras-related protein Rab-5A |
| P23921 | Ribonucleoside-diphosphate reductase large subunit |
| P24666 | Low molecular weight phosphotyrosine protein |
| | phosphatase |
| P31350 | Ribonucleoside-diphosphate reductase subunit M2 |
| P40616 | ADP-ribosylation factor-like protein 1 |
| P50570 | Dynamin-2 |
| P51153 | Ras-related protein Rab-13 |
| P53990 | IST1 homolog |
| P85037 | Forkhead box protein K1 |
| Q00796 | Sorbitol dehydrogenase |
| Q07817 | Bcl-2-like protein 1 |
| Q14134 | Tripartite motif-containing protein 29 |
| Q14807 | Kinesin-like protein KIF22 |
| Q15800 | Methylsterol monooxygenase 1 |
| Q6NZI2 | Caveolae-associated protein 1 |
| Q6PJG6 | BRCA1-associated ATM activator 1 |
| Q6ZRV2 | Protein FAM83H |
| Q6ZXV5 | Protein O-mannosyl-transferase TMTC3 |
| Q70UQ0 | Inhibitor of nuclear factor kappa-B kinase- |
| | interacting protein |
| Q7Z2W4 | Zinc finger CCCH-type antiviral protein 1 |
| Q86U38 | Nucleolar protein 9 |
| Q86V48 | Leucine zipper protein 1 |
| Q8IXK0 | Polyhomeotic-like protein 2 |
| Q8IZW8 | Tensin-4 |
| Q8NC56 | LEM domain-containing protein 2 |
| Q8TEX9 | Importin-4 |
| Q92888 | Rho guanine nucleotide exchange factor 1 |
| Q96HC4 | PDZ and LIM domain protein 5 |
| Q96QD9 | UAP56-interacting factor |
| Q96T76 | MMS19 nucleotide excision repair protein homolog |
| Q99661 | Kinesin-like protein KIF2C |
| Q9BQ69 | ADP-ribose glycohydrolase MACROD1 |
| Q9BW19 | Kinesin-like protein KIFC1 |
| Q9H6R0 | ATP-dependent RNA helicase DHX33 |
| Q9HC35 | Echinoderm microtubule-associated protein-like 4 |
| Q9NVI1 | Fanconi anemia group I protein |
| Q9NZN3 | EH domain-containing protein 3 |
| Q9NZT2 | Opioid growth factor receptor |
| Q9UEY8 | Gamma-adducin |
| Q9UH17 | DNA dC- dU-editing enzyme APOBEC-3B |
| Q9Y639 | Neuroplastin |
| Q9Y6M5 | Zinc transporter 1 |
| O76003 | Glutaredoxin-3 |
| P04183 | Thymidine kinase, cytosolic |
| P29966 | Myristoylated alanine-rich C-kinase substrate |
| P30085 | UMP-CMP kinase |
| P40121 | Macrophage-capping protein |
| P52926 | High mobility group protein HMGI-C |
| P53814 | Smoothelin |
| Q15102 | Platelet-activating factor acetylhydrolase IB |
| | subunit gamma |
| Q16594 | Transcription initiation factor TFIID subunit 9 |
| Q3SXM5 | Inactive hydroxysteroid dehydrogenase-like protein 1 |
| Q53HL2 | Borealin |
| Q71RC2 | La-related protein 4 |
| Q8N183 | NADH dehydrogenase [ubiquinone] 1 alpha |
| | subcomplex assembly factor 2 |
| Q96GD4 | Aurora kinase B |
| Q9H4G4 | Golgi-associated plant pathogenesis-related protein 1 |
| Q9UG63 | ATP-binding cassette sub-family F member 2 |
| Q9UH62 | Armadillo repeat-containing X-linked protein 3 |
| Q9UHA4 | Ragulator complex protein LAMTOR3 |
| Q9UHI6 | Probable ATP-dependent RNA helicase DDX20 |
| Q9UI12 | V-type proton ATPase subunit H |
| Q9Y376 | Calcium-binding protein 39 |

A further analysis of the 1556 proteins identified in the final composition (DP) has shown that 97 of them are of particular interest and can be categorized in 12 superfamilies based on biological, clinical, and cancer prognostic value. Out of these 97 proteins, 52 are overexpressed in the final composition (DP) in comparison to the three cell lines at RCB stage, and 8 are exclusively found in the final composition (DP) but not in any of the RCBs. Table 23 summaries the number of proteins in each of these 12 superfamilies.

TABLE 23
summary of 97 proteins of biological,
clinical, and cancer prognostic value.

| | Total | Over-expressed | Exclusively present |
|---|---|---|---|
| Type | number | in the DP | in the DP |
| Adhesion | 3 | | |
| Antigen | 12 | 5 | |
| ATP-binding | 4 | 4 | 2 |
| cassette | | | |
| BCL | 2 | 2 | 1 |
| COX | 7 | 4 | |
| EGFR | 2 | | |
| HSP | 7 | 5 | |
| Inhibitor | 7 | 2 | 1 |
| MUC | 1 | | |
| RAS-related | 27 | 16 | 2 |
| Repair | 7 | 3 | 1 |
| Transporter | 18 | 11 | 1 |
| | 97 | 52 | 8 |

“Adhesion” corresponds to CAM proteins (cell adhesion molecules), including IgCAMs (such as ICAM1), cadherins, integrins, and selectins.
“ATP binding cassette” corresponds to transmembrane proteins of the transport system superfamily, which are linked with the drug resistance phenomena.
“BCL” corresponds to proteins that regulate cell death, being either pro-apoptotic (such as BAX, BAK1/Bcl-2 homologous antagonist killer, and Bcl-2-associated death promoter) or anti-apoptotic (such as Bcl-2, and Bcl-xL).
“COX” corresponds to cytochrome C oxidase proteins (also termed “complex IV”), which are proteins from the terminal component of the mitochondrial respiratory chain. Mutations in cytochrome C oxidase is involved in cancer (in particular in cytochrome C oxidase subunit 4).
“EGFR” corresponds to epidermal growth factor, involved in the pathogenesis and progression of different carcinoma types.
“HSP” corresponds to heat shock proteins, which are a class of proteins overexpressed in a wide range of human cancers and implicated in tumor cell proliferation, differentiation, invasion, metastasis, death, and recognition by the immune system.
“Inhibitor” corresponds to proteins linked with pro- or anti-cancer proliferation.
“MUC” corresponds to mucin proteins, which are heavily glycosylated proteins. MUC13 in particular is frequently and aberrantly expressed in a variety of epithelial carcinomas, including gastric, colorectal, and ovarian cancers.
“RAS-related” corresponds to Rap GTP-binding proteins, a type of small GTPase. More than 30% of all human cancers - including 95% of pancreatic cancers and 45% of colorectal cancers - are driven by mutations of the RAS family of genes.
“Repair” corresponds to proteins linked with tumor progression.
“Transporter” corresponds to transmembrane proteins with function in drug resistance.

Table 24 identifies these 52 proteins that are overexpressed, among which 8 are exclusively expressed, in the final composition (DP) versus the RCBs.

TABLE 24

| | Uniprot accession | | Membrane | Exclusively expressed |
|---|---|---|---|---|
| Protein class | number | Protein | protein? (Y/N) | in DP? (Y/N) |
| Antigen | P01889 | HLA class I histocompatibility | Y | N |
| | | antigen, B alpha chain | | |
| Antigen | P04439 | HLA class I histocompatibility | Y | N |
| | | antigen, A alpha chain | | |
| Antigen | P10321 | HLA class I histocompatibility | Y | N |
| | | antigen, C alpha chain | | |
| Antigen | P21926 | CD9 antigen | Y | N |
| Antigen | P23497 | Nuclear autoantigen Sp-100 | N | N |
| ATP-binding | P28288 | ATP-binding cassette sub- | Y | N |
| cassette | | family D member 3 | | |
| ATP-binding | P61221 | ATP-binding cassette sub- | N | N |
| cassette | | family E member 1 | | |
| ATP-binding | Q8NE71 | ATP-binding cassette sub- | N | Y |
| cassette | | family F member 1 | | |
| ATP-binding | Q9UG63 | ATP-binding cassette sub- | N | Y |
| cassette | | family F member 2 | | |
| BCL | Q07817 | Bcl-2-like protein 1 | Y | Y |
| BCL | Q9NYF8 | Bcl-2-associated transcription | N | N |
| | | factor 1 | | |
| COX | P00403 | Cytochrome c oxidase subunit 2 | Y | N |
| COX | P13073 | Cytochrome c oxidase subunit 4 | Y | N |
| | | isoform 1, mitochondrial | | |
| COX | P20674 | Cytochrome c oxidase subunit | N | N |
| | | 5A, mitochondrial | | |
| COX | Q15067 | Peroxisomal acyl-coenzyme | N | N |
| | | A oxidase 1 | | |
| HSP | P04792 | Heat shock protein beta-1 | N | N |
| HSP | P08238 | Heat shock protein HSP 90-beta | N | N |
| HSP | P11142 | Heat shock cognate 71 kDa protein | N | N |
| HSP | P17066 | Heat shock 70 kDa protein 6 | N | N |
| HSP | P34932 | Heat shock 70 kDa protein 4 | N | N |
| Inhibitor | P13489 | Ribonuclease inhibitor | N | N |
| Inhibitor | Q70UQ0 | Inhibitor of nuclear factor kappa- | Y | Y |
| | | B kinase-interacting protein | | |
| RAS related | P20339 | Ras-related protein Rab-5A | N | Y |
| RAS related | P20340 | Ras-related protein Rab-6A | N | N |
| RAS related | P51148 | Ras-related protein Rab-5C | N | N |
| RAS related | P51149 | Ras-related protein Rab-7a | N | N |
| RAS related | P51153 | Ras-related protein Rab-13 | N | Y |
| RAS related | P57735 | Ras-related protein Rab-25 | N | N |
| RAS related | P59190 | Ras-related protein Rab-15 | N | N |
| RAS related | P61006 | Ras-related protein Rab-8A | N | N |
| RAS related | P61026 | Ras-related protein Rab-10 | N | N |
| RAS related | P61224 | Ras-related protein Rap-1b | N | N |
| RAS related | P62820 | Ras-related protein Rab-1A | N | N |
| RAS related | P62834 | Ras-related protein Rap-1A | N | N |
| RAS related | P63000 | Ras-related C3 botulinum | N | N |
| | | toxin substrate 1 | | |
| RAS related | Q92930 | Ras-related protein Rab-8B | N | N |
| RAS related | Q9NP72 | Ras-related protein Rab-18 | N | N |
| RAS related | Q9Y3L5 | Ras-related protein Rap-2c | N | N |
| Repair | P12956 | X-ray repair cross- | N | N |
| | | complementing protein 6 | | |
| Repair | P52701 | DNA mismatch repair | N | N |
| | | protein Msh6 | | |
| Repair | Q96T76 | MMS19 nucleotide excision | N | Y |
| | | repair protein homolog | | |
| Transporter | O15027 | Protein transport protein Sec16A | N | N |
| Transporter | P05023 | Sodium/potassium-transporting | Y | N |
| | | ATPase subunit alpha-1 | | |
| Transporter | P11166 | Solute carrier family 2, facilitated | Y | N |
| | | glucose transporter member 1 | | |
| Transporter | P53007 | Tricarboxylate transport | Y | N |
| | | protein, mitochondrial | | |
| Transporter | P53985 | Monocarboxylate transporter 1 | Y | N |
| Transporter | P60468 | Protein transport protein | Y | N |
| | | Sec61 subunit beta | | |
| Transporter | P61619 | Protein transport protein | Y | N |
| | | Sec61 subunit alpha isoform 1 | | |
| Transporter | Q5JRA6 | Transport and Golgi organization | Y | N |
| | | protein 1 homolog | | |
| Transporter | Q8TB61 | Adenosine 3′-phospho 5′- | Y | N |
| | | phosphosulfate transporter 1 | | |
| Transporter | Q92973 | Transportin-1 | N | N |
| Transporter | Q9Y6M5 | Zinc transporter 1 | Y | Y |

Table 25 shows the implication of these 97 proteins of biological, clinical, and cancer prognostic value in various types of cancers. As seen, the final composition (DP) expresses markers that are not only linked to colorectal cancer, as could be expected given that HT-29, HCT-116 and LoVo cells are colorectal cell lines, but also to other types of cancers. This suggests that the composition described herein could be useful for treating not only colorectal cancer but also a wide variety of other cancers.

TABLE 25

| | Number of |
|---|---|
| Cancer type | identified markers |
| Breast | 6 |
| Colorectal | 8 |
| Liver | 37 |
| Renal | 48 |
| Pancreatic | 15 |
| Endometrial | 13 |
| Head and neck | 4 |
| Ovarian | 8 |
| Lung | 6 |
| Cervical | 2 |
| Melanoma | 1 |
| Glioma | 1 |

### Example 5

### LC-MS/MS Identification and Relative Quantification of Surface Proteins in 2 Intermediate Compositions and in the Final Product

This study aimed at characterizing the surface proteome of tumoral cells at different production steps and finally identifying differentially expressed surface proteins in one of the starting materials (HT-29 MCB), in the 2 corresponding intermediate compositions (HT-29 DS-A and HT-29 DS-B) and in the final product (DP) comprising all 6 DS described above (hence including the HT-29 DS-A and HT-29 DS-B).

### Material and Methods

Similar type of product (DS/DP) as those described in the previous examples 3 & 4 have been analyzed. The same steps have been applied specifically exposures to different stresses (radiations, thermal stress, chemical stress, metabolic stress or combinations thereof). These different stresses induced an overexpression of antigens. Immunogenicity has also been enhanced by chemically marking the surface proteins with haptens. Protein expression was compared between different cell samples after exposure to different stresses. A summary of the different compositions analyzed in this study is given in Table 26.

TABLE 26

| | | | Cell |
|---|---|---|---|
| Sample | | Volume | concentration |
| identification | Product | (mL) | (cells/mL) |
| HT-29 MCB | HT-29 cultured in McCoy's | 1 | 6 × 10⁶ |
| | 5A, 2% FBS, 10% DMSO | | |
| HT-29 DS-A | HT-29 in EBSS, saccharose, 5% | 0.5 | 3.88 × 10⁷ |
| | DMSO, stressed by radiation and | | |
| | thermic choc, then haptenated | | |
| HT-29 DS-B | HT-29 in EBSS, saccharose, 5% | 0.15 | 2.6 × 10⁷ |
| | DMSO, stressed by chemical stress, | | |
| | then haptenated | | |
| DP | Drug product comprising HT-29 DS-A | 2 | 3 × 10⁶ |
| | and HT-29 DS-B, together with | | |
| | HCT-116 DS-A, HCT-116 DS-B, | | |
| | LoVo DS-A and LoVo DS-B | | |

### Samples Treatment

When possible, an amount of 6×10⁶ cells (otherwise, whole sample was used) were gently washed with PBS and were then biotinylated using Pierce™ Cell Surface Protein Biotinylation. Cell were then lysed and proteins were isolated using Pierce™ Cell Surface Protein Biotinylation and Isolation Kit (Thermo Scientific, Catalog Numbers A44390). Both preparations steps were performed according to manufacturer's instructions.

Proteins were then precipitated with methanol-chloroform, precipitates were washed with methanol, dried and solubilized in iST LYSE (PreOmics Gmbh) buffer by micro-cavitation (Bioruptor Pico, Diagenode).

Proteins were digested using LysC and trypsin. Peptides were purified using a mixed-mode reverse phase cation exchanger SPE column (PreOmics Gmbh), dried and solubilized in 100 μL of 3% acetonitrile 0.1% formic acid aqueous solution.

Peptides concentration was determined using BCA method (Table 27). Despite low measured peptides concentrations, their quantities were sufficient for this feasibility study.

TABLE 27

| Sample | Mean peptide |
|---|---|
| identification | concentration (μg/μL) |
| HT-29 MCB | 0.029 |
| HT-29 DS-A | 0.050 |
| HT-29 DS-B | 0.049 |
| DP | 0.048 |

### Analytical Method

LC-MS/MS

250 ng of peptides were injected in triplicate for each sample.

Chromatography was performed using an Ultimate 3000 (Dionex) equipment using PepMap100 C18 (75 μm×50 cm, 2 μm material) column applying a 2.5%-to-35% acetonitrile 120-minute gradient at a flow rate of 300 nL/minute after a 3-minute trapping step on precolumn.

Data were acquired using a Q-Exactive (Thermo) mass spectrometer using experimental settings described in Table 28. MS/MS scan was performed on the 10 most intense ions of each cycle, 6545 cycles were performed, thus an average of 17 cycles per chromatographic peak.

TABLE 28

| | | Sweep | Transfer tube |
|---|---|---|---|
| Source | Voltage | gas | temperature |
| | 1900V | 0 psi | 275° C. |

| Mass | | Accumulation | Normalized |
|---|---|---|---|
| spectrometer | Resolution | time | collision energy |
| MS scan | 70 000 | 60 ms | — |
| MS/MS scan | 17 500 | 60 ms | 28 |

Protein Identification

Data were processed with Proteome Discoverer 2.4.

Proteins were identified using the SEQUEST-HT algorithm against a database gathering human reference proteome mined from NeXtProt and cRAP contaminant database depleted of human proteins.

Search parameters were:
enzyme=trypsin (full);
allowed miscleavage=2;
precursor error tolerance=10 ppm;
fragment error tolerance=0.02 Da;
dynamic modification=oxidation (M), deamidation (N/Q), CAMthiopropanoyl (K);
protein terminus modification=acetylation, CAMthiopropan oyl;
static modification=carbamidomethI(C).

False Discovery Rate (FDR) determination was made using Percolator algorithm.

All spectra reported with a confidence less than high by SEQUEST-HT, i.e., considered as not identified, were processed a second time by the same algorithm against the same database than above, but using modified settings:
enzyme=trypsin_R (semi);
allowed miscleavage=2;
precursor error tolerance=10 ppm;
fragment error tolerance=0.02 Da;
dynamic modification=oxidation (M), deamidation (N/Q), CAMthiopropanoyl (K);
protein terminus modification=acetylation, CAMthiopropan oyl;
static modification=carbamidomethI(C).

Flase Discovery Rate (FDR) determination was made using Percolator algorithm.

Proteins were considered as part of “membrane” or “cell surface” when these keywords were reported as annotations in the “cellular component” field of the Thermo Protein Center database. It has to be noted that several cellular components may be reported for the same protein. Furthermore, the term “membrane” can refer to membrane other than plasma membrane (e.g., nucleus membrane, organelle membrane, vesicle membrane, etc.).

### Protein Quantification

Data were processed using Minora and feature mapper for Proteome Discoverer 2.4 software.

Peak integration parameters were:
post-acquisition recalibration=true (fine parameters);
minimum trace length=5;
max delta RT for isotope=0.2 minutes;
PSM confidence level for integration=high.

Chromatographic alignment parameters were:
RT alignment=true;
parameter tuning=fine;
max RT shift=5 minutes;
mass tolerance=10 ppm.

Feature mapping parameters were:
RT tolerance=automatic;
mass tolerance=automatic;
S/N threshold=2.

Statistical analyses were performed by using Precursors Ions quantifier node for Proteome Discoverer 2.4 software.

General quantification settings were:
peptide to use=unique+RAZOR (unique meaning peptides that are not shared by different proteins or protein groups; RAZOR meaning peptides shared by multiple protein's groups but only used to quantify protein with the largest number of unique peptides and with the longest amino acid sequence);
consider proteins groups for peptide uniqueness=true;
reject quan results with missing channels=false.

Precursor quantification settings were:
precursor abundance based on=area;
min number replicate feature=50% (peptides must be detected in at least 50% of sample of one group for be use in quantification).

Normalization settings were:
normalization mode=total Peptide amount (calculates the total sum of abundance values for each injection over all peptides identified, the injection with the highest total abundance is used as reference to correct abundance values in all other injections by a constant factor per injection, so that at the end the total abundance is the same for all injections).

Quan rollup hypothesis testing settings were:
ratio calculation=pairwise ratio-based (peptides ratios are calculated as geometric median of all combinations ratio from all replicates for selected study factor. The proteins ratio is subsequently calculated as geometric median of peptides group ratio);
Imputation mode=replicate-based resampling (missing values are replaced with random values sampled from distributions centered around medians of detected values of (technical, biological) replicates);
hypothesis test=t-test (background-based).

The hypothesis test giving the p-value is a t-background test (or ANOVA background). This test has been based on the assumption that most protein abundances do not vary in response to stimulus, in proteomics. This method determines a rank of protein ratios considered mainly constant between conditions before testing each protein abundance ratio against median and variance of this constant population. This test is useful with studies having missing values and can be used only when hundreds of proteins are identified. It does not require technical replicate.

### Results

### Number of Proteins Detection

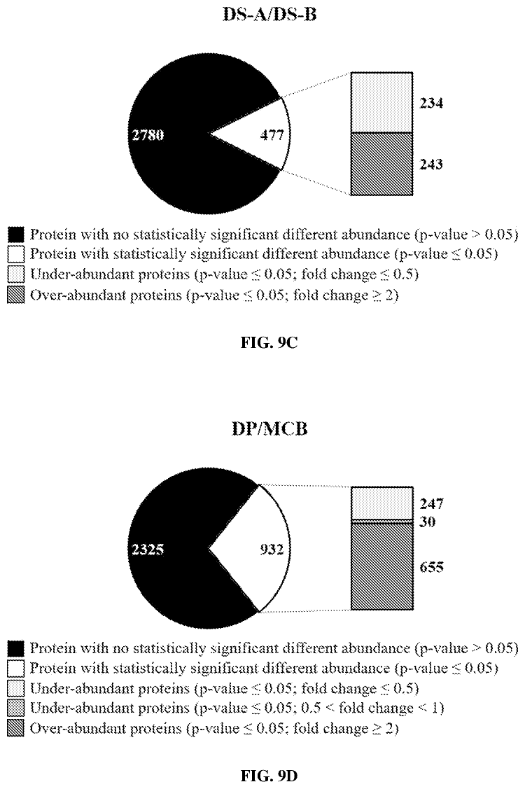

The number of proteins and peptides identified in each sample are displayed in Table 29 and FIG. 8.

TABLE 29

| | PEP: number of peptides |
|---|---|
| Sample identification | PRO: number of proteins |
| HT-29 MCB | EP: 5 828 |
| | RO: 1 418 |
| HT-29 DS-A | EP: 15 368 |
| | RO: 2 373 |
| HT-29 DS-B | EP: 13 928 |
| | RO: 2 290 |
| DP | EP: 10 121 |
| | RO: 1 758 |

### Relative Quantification of Proteins

Four comparisons were done to compare:
HT-29 DS-A to HT-29 DS-B,
HT-29 DS-A to HT-29 MCB,
HT-29 DS-B to HT-29 MCB, and
HT-29 MCB to DP.

Note 1: due to biological or technical variations and/or the stochastic nature of mass spectrometric acquisition of trace data, some protein abundance values may be lost. Thus, we consider as quantifiable, proteins whose quantifiable peptides are present in at least 50% of the injections in either group.

Note 2: for some proteins or peptides, the abundance ratios are equal to 1000 or 0.001. These values are arbitrary. In the first case, they mean that this protein or peptide has been quantified only in the numerator condition; in the second case, only in the denominator condition.

Note 3: proteins not identified on the basis of their peptides' MS/MS fragmentation spectra can still be quantified through “match between run”. This quantification is based on the similarity of the XIC (MS1) chromatographic characteristics of their peptides (exact mass, retention time) with those of peptides identified in at least one acquisition of the same experiment.

Note 4: protein abundance is considered statistically and significantly different when the associated p-value is less than or equal to 0.05 with an abundance ratio less than or equal to 0.5 or greater than or equal to 2.

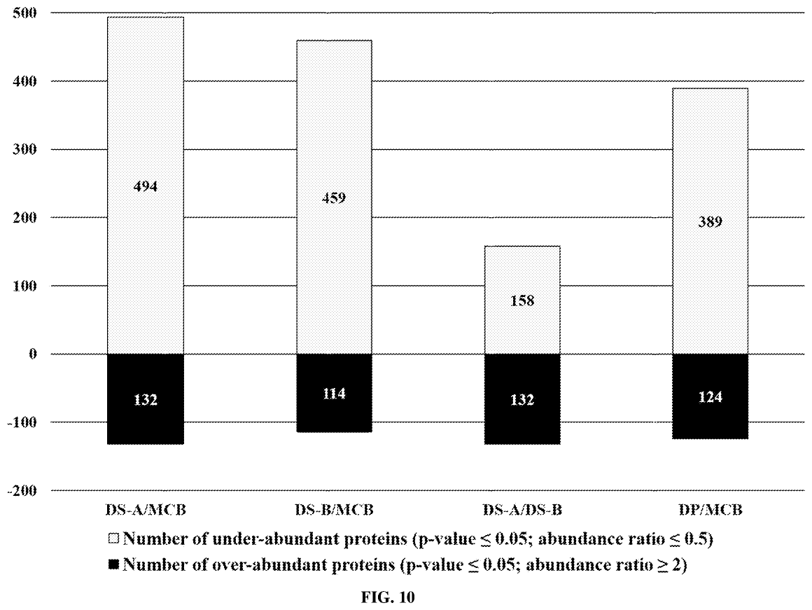

FIG. 9A-D show the proteins distribution according to p-values and fold changes in each of the four comparisons.

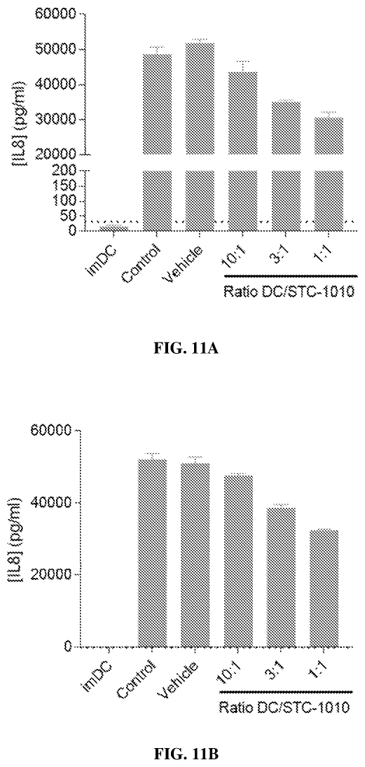

FIG. 10 shows the number of proteins from membrane and/or cell surface with statistical and significant difference in each of the four comparisons.

For all injections, more than 65% of the proteins identified were associated to the terms “membrane” or “cell surface” in the “cellular component” field of the Thermo Protein Center database (FIG. 8). As a comparison, in non-biotinylated HeLa cells, a lower proportion of identified proteins (51%, i.e., 816 proteins out of 3562) are associated to the terms “membrane” or “cell surface” in the same database. This tends to indicate successful enrichment of surface proteome in all four samples.

Relative quantification and comparisons between samples showed that abundances of about 29% to 34% of proteins significantly vary when stressed/haptenated cells (DS-A or DS-B) or drug product (DP) are compared to non-treated cells (MCB) (FIG. 9A-C, respectively). A lower proportion (15%) is observed when both stressed/haptenated cell samples (DS-A and DS-B) are compared to each other (FIG. 9D).

More than one half of significantly over- or under-expressed proteins are annotated as membrane and cell surface proteins (FIG. 10).

### Comparison Results

HT-29 DS-A/HT-29 MCB

Based on the raw data, an abundance ratio DS-A/MCB≥1000 has been selected to sort the overexpressed membrane or cell surface proteins after a stress by radiation and thermic choc, followed by haptenation.

Considering the cells membranes proteins, 455 proteins were identified as overexpressed in the DS-A compared to the MCB with an abundance ratio ≥1000.

Considering the specific cell surface proteins, 127 proteins were identified as overexpressed in the DS-A compared to the MCB with an abundance ratio ≥2, and 38 of them with an abundance ratio of 1000 (Table 30).

TABLE 30

| Uniprot accession number | Protein |
|---|---|
| P54652 | Heat shock-related 70 kDa protein |
| O76027 | Annexin |
| Q4KMQ2 | Anoctamin-6 |
| O75054 | Immunoglobulin superfamily member 3 |
| P02787 | Serotransferrin |
| O14763 | Tumor necrosis factor receptor superfamily member 10B |
| P10909 | Clusterin |
| P09958 | Furin |
| P04233 | HLA class II histocompatibility antigen gamma chain |
| Q6YHK3 | CD109 antigen |
| Q9Y696 | Chloride intracellular channel protein 4 |
| Q14517 | Protocadherin Fat 1 |
| P49281 | Natural resistance-associated macrophage protein 2 |
| O00220 | Tumor necrosis factor receptor superfamily member 10A |
| O15484 | Calpain-5 |
| Q29983 | MHC class I polypeptide-related sequence A |
| P09429 | High mobility group protein B1 |
| O95858 | Tetraspanin-15 |
| Q9BZM5 | UL16-binding protein 2 |
| P26010 | Integrin beta-7 |
| Q15465 | Sonic hedgehog protein |
| O15455 | Toll-like receptor 3 |
| P02749 | Beta-2-glycoprotein 1 |
| P13726 | Tissue factor |
| P29122 | Proprotein convertase subtilisin/kexin type 6 |
| Q9UNN8 | Endothelial protein C receptor |
| Q8IWT6 | Volume-regulated anion channel subunit LRRC8A |
| Q9NYQ7 | Cadherin EGF LAG seven-pass G-type receptor 3 |
| Q13433 | Zinc transporter ZIP6 |
| P28067 | HLA class II histocompatibility antigen, DM alpha chain |
| Q9UPY5 | Cystine/glutamate transporter |
| Q9HBW0 | Lysophosphatidic acid receptor 2 |
| P18827 | Syndecan-1 |
| Q12891 | Hyaluronidase-2 |
| P13612 | Integrin alpha-4 |
| P04196 | Histidine-rich glycoprotein |
| P01137 | Transforming growth factor beta-1 proprotein |
| P16035 | Metalloproteinase inhibitor 2 |

HT-29 DS-AIHT-29 MCB

Based on the raw data, an abundance ratio DS-B/MCB≥1000 has been selected to sort the overexpressed membrane or cell surface proteins after a chemical choc, followed by haptenation.

Considering the cells membranes proteins, 430 proteins were identified as overexpressed in the DS-B compared to the MCB with an abundance ratio ≥1000.

Considering the specific cell surface proteins, 127 proteins were identified as overexpressed in the DS-B compared to the MCB with an abundance ratio ≥2 and 37 of them with an abundance ratio of 1000 (Table 31).

TABLE 31

| Uniprot accession number | Protein |
|---|---|
| Q6YHK3 | CD109 antigen |
| P04233 | HLA class II histocompatibility antigen gamma chain |
| P30511 | HLA class I histocompatibility antigen, alpha chain F |
| O75330 | Hyaluronan mediated motility receptor |
| P26012 | Integrin beta-8 |
| P05106 | Integrin beta-3 |
| P29122 | Proprotein convertase subtilisin/kexin type 6 |
| P10909 | Clusterin |
| P02787 | Serotransferrin |
| P49281 | Natural resistance-associated macrophage protein 2 |
| Q29983 | MHC class I polypeptide-related sequence A |
| O14763 | Tumor necrosis factor receptor superfamily member 10B |
| Q9UNN8 | Endothelial protein C receptor |
| Q9NYQ7 | Cadherin EGF LAG seven-pass G-type receptor 3 |
| O00220 | Tumor necrosis factor receptor superfamily member 10A |
| Q9UPY5 | Cystine/glutamate transporter |
| P13726 | Tissue factor |
| P01137 | Transforming growth factor beta-1 proprotein |
| O75054 | Immunoglobulin superfamily member 3 |
| Q4KMQ2 | Anoctamin-6 |
| P16035 | Metalloproteinase inhibitor 2 |
| O15455 | Toll-like receptor 3 |
| Q8IWT6 | Volume-regulated anion channel subunit LRRC8A |
| O95858 | Tetraspanin-15 |
| Q13433 | Zinc transporter ZIP6 |
| P09958 | Furin |
| Q14517 | Protocadherin Fat 1 |
| Q12891 | Hyaluronidase-2 |
| Q9HBW0 | Lysophosphatidic acid receptor 2 |
| P09429 | High mobility group protein B1 |
| Q9Y696 | Chloride intracellular channel protein 4 |
| Q9BZM5 | UL16-binding protein 2 |
| O15484 | Calpain-5 |
| O76027 | Annexin A9 |
| P04196 | Histidine-rich glycoprotein |
| P13612 | Integrin alpha-4 |
| P54652 | Heat shock-related 70 kDa protein 2 |

DP/HT-29 MCB

Note: this comparison is not representative of the real protein overexpression due to the composition of the DP that gatherers HT-29 DS-A and HT-29 DS-B, but also HCT-116 DS-A, HCT-116 DS-B, LoVo DS-A and LoVo DS-B; hence, a dilution factor would need to be taken account for the MCB comparison.

Based on the raw data, an abundance ratio DP/MCB ≥1000 has been selected to sort the overexpressed membrane or cell surface proteins.

Considering the cells membranes proteins, 343 proteins were identified as overexpressed in the DP compared to the MCB with an abundance ratio ≥1000.

Considering the specific cell surface proteins, 112 proteins were identified as overexpressed in the DP compared to the MCB with an abundance ratio >2, and 34 of them with an abundance ratio of 1000 (Table 32).

TABLE 32

| Uniprot accession number | Protein |
|---|---|
| P02749 | Beta-2-glycoprotein 1 |
| P28067 | HLA class II histocompatibility antigen, DM alpha chain |
| Q6YHK3 | CD109 antigen |
| P04233 | HLA class II histocompatibility antigen gamma chain |
| P30511 | HLA class I histocompatibility antigen, alpha chain F |
| O75330 | Hyaluronan mediated motility receptor |
| P26012 | Integrin beta-8 |
| P05106 | Integrin beta-3 |
| P29122 | Proprotein convertase subtilisin/kexin type 6 |
| P10909 | Clusterin |
| P02787 | Serotransferrin |
| Q29983 | MHC class I polypeptide-related sequence A |
| O14763 | Tumor necrosis factor receptor superfamily member 10B |
| Q9UNN8 | Endothelial protein C receptor |
| Q9NYQ7 | Cadherin EGF LAG seven-pass G-type receptor 3 |
| O00220 | Tumor necrosis factor receptor superfamily member 10A |
| P13726 | Tissue factor |
| O75054 | Immunoglobulin superfamily member 3 |
| Q4KMQ2 | Anoctamin-6 |
| O15455 | Toll-like receptor 3 |
| O95858 | Tetraspanin-15 |
| Q13433 | Zinc transporter ZIP6 |
| P09958 | Furin |
| Q14517 | Protocadherin Fat 1 |
| Q9HBW0 | Lysophosphatidic acid receptor 2 |
| Q9Y696 | Chloride intracellular channel protein 4 |
| Q9BZM5 | UL16-binding protein 2 |
| O15484 | Calpain-5 |
| O76027 | Annexin A9 |
| P04196 | Histidine-rich glycoprotein |
| P13612 | Integrin alpha-4 |
| P54652 | Heat shock-related 70 kDa protein 2 |
| P08648 | Integrin alpha-5 |
| P43003 | Excitatory amino acid transporter 1 |

### Proteome Identification in the Drug Product (DP)

Table 33 describes the 132 cell surface proteins identified in 3 triplicates of the DP comprising a mix of HT-29 DS-A, HT-29 DS-B, HCT-116 DS-A, HCT-116 DS-B, LoVo DS-A and LoVo DS-B.

TABLE 33

| Uniprot accession number | Protein | Gene symbol |
|---|---|---|
| P10809 | 60 kDa heat shock protein, mitochondrial | HSPD1 |
| P16422 | Epithelial cell adhesion molecule | EPCAM |
| P08238 | Heat shock protein HSP 90-beta | HSP90AB1 |
| P11279 | Lysosome-associated membrane glycoprotein 1 | LAMP1 |
| P13688 | Carcinoembryonic antigen-related cell adhesion molecule 1 | CEACAM1 |
| P05362 | Intercellular adhesion molecule 1 | ICAM1 |
| P25445 | Tumor necrosis factor receptor superfamily member 6 | FAS |
| P06576 | ATP synthase subunit beta, mitochondrial | ATP5B |
| P11021 | Endoplasmic reticulum chaperone BiP | HSPA5 |
| P13667 | Protein disulfide-isomerase A4 | PDIA4 |
| P30101 | Protein disulfide-isomerase A3 | PDIA3 |
| P27797 | Calreticulin | CALR |
| P02786 | Transferrin receptor protein 1 | TFRC |
| P05187 | Alkaline phosphatase, placental type | ALPP |
| P17301 | Integrin alpha-2 | ITGA2 |
| P07355 | Annexin A2 | ANXA2 |
| O15031 | Plexin-B2 | PLXNB2 |
| P11717 | Cation-independent mannose-6-phosphate receptor | IGF2R |
| P08195 | 4F2 cell-surface antigen heavy chain | SLC3A2 |
| P06756 | Integrin alpha-V | ITGAV |
| Q9HDC9 | Adipocyte plasma membrane-associated protein | APMAP |
| Q9BS26 | Endoplasmic reticulum resident protein 44 | ERP44 |
| P06733 | Alpha-enolase | ENO1 |
| P05556 | Integrin beta-1 | ITGB1 |
| Q12907 | Vesicular integral-membrane protein VIP36 | LMAN2 |
| P09525 | Annexin A4 | ANXA4 |
| P30040 | Endoplasmic reticulum resident protein 29 | ERP29 |
| P2158′ | 5′-nucleotidase | NT5E |
| P26006 | Integrin alpha-3 | ITGA3 |
| P00505 | Aspartate aminotransferase, mitochondrial | GOT2 |
| Q96JJ7 | Protein disulfide-isomerase TMX3 | TMX3 |
| Q07021 | Complement component 1 Q subcomponent- | C1QBP |
| | binding protein, mitochondrial | |
| P04439 | HLA class I histocompatibility antigen, A alpha chain | HLA-A |
| P50895 | Basal cell adhesion molecule | BCAM |
| P18084 | Integrin beta-5 | ITGB5 |
| Q8TCT9 | Minor histocompatibility antigen H13 | HM13 |
| P35232 | Prohibitin OS × Homo sapiens (Human) | PHB |
| Q99623 | Prohibitin-2 OS × Homo sapiens (Human) | PHB2 |
| P23229 | Integrin alpha-6 | ITGA6 |
| Q92692 | Nectin-2 | PV RL2; |
| | | NECTIN2 |
| P27487 | Dipeptidyl peptidase 4 | DPP4 |
| P04083 | Annexin A1 | ANXA1 |
| P01893 | Putative HLA class I histocompatibility | HLA-H |
| | antigen, alpha chain H | |
| P16070 | CD44 antigen | CD44 |
| P10321 | HLA class I histocompatibility antigen, C alpha chain | HLA-C |
| Q96PD2 | Discoidin, CUB and LCCL domain-containing protein 2 | DCBLD2 |
| Q6UVK1 | Chondroitin sulfate proteoglycan 4 | CSPG4 |
| P01889 | HLA class I histocompatibility antigen, B alpha chain | HLA-B |
| P08582 | Melanotransferrin | M FI2; |
| | | MELTF |
| Q14114 | Low-density lipoprotein receptor-related protein 8 | LRP8 |
| P16144 | Integrin beta-4 | ITGB4 |
| P01130 | Low-density lipoprotein receptor | LDLR |
| Q12913 | Receptor-type tyrosine-protein phosphatase eta | PTPRJ |
| P30533 | Alpha-2-macroglobulin receptor-associated protein | LRPAP1 |
| Q99523 | Sortilin | SORT1 |
| Q15836 | Vesicle-associated membrane protein 3 | VAMP3 |
| P00533 | Epidermal growth factor receptor | EGFR |
| P05067 | Amyloid-beta precursor protein | APP |
| P15151 | Poliovirus receptor | PVR |
| Q13641 | Trophoblast glycoprotein | TPBG |
| P11233 | Ras-related protein Ral-A | RALA |
| P56199 | Integrin alpha-1 | ITGA1 |
| P07225 | Vitamin K-dependent protein S | PROS1 |
| Q9Y639 | Neuroplastin | NPTN |
| Q14126 | Desmoglein-2 | DSG2 |
| P61769 | Beta-2-microglobulin | B2M |
| P48960 | CD97 antigen | C D97; |
| | | ADGRE5 |
| P10909 | Clusterin | CLU |
| O43490 | Prominin-1 | PROM1 |
| O14763 | Tumor necrosis factor receptor superfamily member 10B | TNFRSF10B |
| Q6YHK3 | CD109 antigen | CD109 |
| Q12846 | Syntaxin-4 | STX4 |
| P09382 | Galectin-1 | LGALS1 |
| P78536 | Disintegrin and metalloproteinase domain- | ADAM17 |
| | containing protein 17 | |
| Q9UBR2 | Cathepsin Z | CTSZ |
| P19013 | Keratin, type II cytoskeletal 4 | KRT4 |
| P14735 | Insulin-degrading enzyme | IDE |
| P19075 | Tetraspanin-8 | TSPAN8 |
| Q8WTV0 | Scavenger receptor class B member 1 | SCARB1 |
| P17813 | Endoglin | ENG |
| O14672 | Disintegrin and metalloproteinase domain- | ADAM10 |
| | containing protein 10 | |
| Q10589 | Bone marrow stromal antigen 2 | BST2 |
| P43007 | Neutral amino acid transporter A | SLC1A4 |
| Q9P2B2 | Prostaglandin F2 receptor negative regulator | PTGFRN |
| P08962 | CD63 antigen | CD63 |
| P15291 | Beta-1,4-galactosyltransferase 1 | B4GALT1 |
| P13987 | CD59 glycoprotein | CD59 |
| O00220 | Tumor necrosis factor receptor superfamily member 10A | TNFRSF10A |
| O94985 | Calsyntenin-1 | CLSTN1 |
| O95297 | Myelin protein zero-like protein 1 | MPZL1 |
| P08648 | Integrin alpha-5 | ITGA5 |
| P51809 | Vesicle-associated membrane protein 7 | VAMP7 |
| Q14517 | Protocadherin Fat 1 | FAT1 |
| P32004 | Neural cell adhesion molecule L1 | LICAM |
| Q9Y696 | Chloride intracellular channel protein 4 | CLIC4 |
| Q29983 | MHC class I polypeptide-related sequence A | MICA |
| Q9UII2 | ATPase inhibitor, mitochondrial | ATPIF1 |
| Q9H0X4 | Protein FAM234A | IT FG3; |
| | | FAM234A |
| Q9BZM5 | UL16-binding protein 2 | ULBP2 |
| O15258 | Protein RER1 | RER1 |
| P08174 | Complement decay-accelerating factor | CD55 |
| P29317 | Ephrin type-A receptor 2 | EPHA2 |
| P19021 | Peptidyl-glycine alpha-amidating monooxygenase | PAM |
| Q03405 | Urokinase plasminogen activator surface receptor | PLAUR |
| P19256 | Lymphocyte function-associated antigen 3 | CD58 |
| P02749 | Beta-2-glycoprotein 1 | APOH |
| P15529 | Membrane cofactor protein | CD46 |
| P02787 | Serotransferrin | TF |
| Q13444 | Disintegrin and metalloproteinase domain- | ADAM15 |
| | containing protein 15 | |
| P49810 | Presenilin-2 | PSEN2 |
| P04156 | Major prion protein | PRNP |
| P13726 | Tissue factor | F3 |
| P21926 | CD9 antigen | CD9 |
| Q13433 | Zinc transporter ZIP6 | SLC39A6 |
| Q4KMQ2 | Anoctamin-6 | ANO6 |
| P08581 | Hepatocyte growth factor receptor | MET |
| Q9UNN8 | Endothelial protein C receptor | PROCR |
| P02788 | Lactotransferrin | LTF |
| P19634 | Sodium/hydrogen exchanger 1 | SLC9A1 |
| P09958 | Furin OS × Homo sapiens (Human) | FURIN |
| P30511 | HLA class I histocompatibility antigen, alpha chain F | HLA-F |
| P31431 | Syndecan-4 | SDC4 |
| Q14210 | Lymphocyte antigen 6D | LY6D |
| P14174 | Macrophage migration inhibitory factor | MIF |
| O75054 | Immunoglobulin superfamily member 3 | IGSF3 |
| Q9NYQ7 | Cadherin EGF LAG seven-pass G-type receptor 3 | CELSR3 |
| P21810 | Biglycan | BGN |
| P43003 | Excitatory amino acid transporter 1 | SLC1A3 |
| P13612 | Integrin alpha-4 | ITGA4 |
| Q13492 | Phosphatidylinositol-binding clathrin assembly protein | PICALM |
| P15328 | Folate receptor alpha | FOLR1 |
| Q00839 | Heterogeneous nuclear ribonucleoprotein U | HNRNPU |

### Example 6

### In Vitro Evaluation of the Functional Activity of STC-1010 Through Mixed Lymphocyte Reaction Assay

The aim was to assess in vitro the final product (DP or “STC-1010”), for its potential to induce an immunogenic profile of human monocytes-derived dendritic cells (DCs) when applied alone or in combination with CD40L (naturally present in vivo) and to evaluate, in a mixed lymphocyte reaction (MLR) assay, the T cell activation mediated by dendritic cells. The study was performed on a co-culture of HLA-matched monocyte-derived mDCs and CD8⁺ T cells, in which system i) the cytokine profile of DCs was measured through the quantitation of cytokines released in the culture supernatants (including IL-12 and IL-8), and ii) the DC-mediated T cell activation was measured through the assessment of the MLR response by the mean of the quantification of released IFNγ using specific homogeneous time-resolved fluorescence (HTRF)-based detection kits.

### Materials and Methods

Human PBMCs from healthy donors were used in this study in order to isolate freshly i) monocytes which were used to obtain mature DCs through differentiation/maturation protocols, as well as ii) CD8⁺ lymphocytes.

Briefly, human monocytes were freshly isolated from PBMCs and were differentiated into DCs under cultivation in the presence of GM-CSF and IL-4. At the end of the differentiation process, DCs were matured in a specific cocktail in the presence of LPS and IFNγ containing STC-1010 at three different ratios (1:1, 3:1 and 10:1, plus a condition without STC-1010 as negative control) in the presence and absence of 0.6 μg/mL CD40L.

Upon maturation, mDCs were validated as CD209⁺ CD1a⁺ CD80⁺ CD83⁺ CD86⁺ and cell culture supernatants were retrieved for IL-12 and IL-8 quantitation by means of HTRF.

Then, DCs were co-cultured, at an appropriate stimulator:responder ratio of 1:4, with CD8⁺ T cells isolated from the same donor. Control conditions (untreated), as well as T cells alone and mDCs alone, were included in the experiment. 72 hours following co-culture, supernatants were collected and effects of STC-1010 were evaluated on CD8+T cell activation by mean of the quantification of released IFNγ levels that was used as a key representative surrogate of T cell activation. Cytokine level quantification was performed by HTRF.

### Results

### Effects of STC-1010 on mDC Cytokines

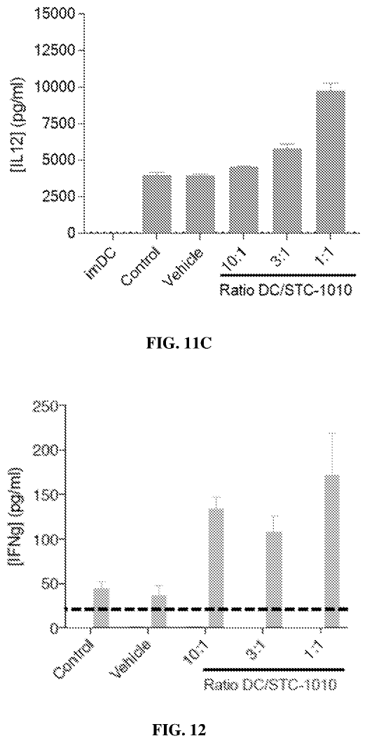

STC-1010 was able to limit in a ratio-dependent manner the secretion of IL-8 by mDCs, both in absence and in presence of CD40L (FIGS. 11A and 11B, respectively). STC-1010 was also able to enhance in a ratio-dependent manner the secretion of IL-12 by mDCs in presence of CD40L (FIG. 11C). This ratio-dependent response confirms the action of STC-1010 on the maturation of DCs.

Two other cytokines (IL-10 and TNFα) were also evaluated in the course of this study: STC-1010 demonstrated a trend to limit IL-10 secretion in the presence of CD40L; while TNFα secretion was slightly decreased upon STC-1010 exposure with an effect culminating at the highest ratio, both in absence or presence of CD40L (data not shown).

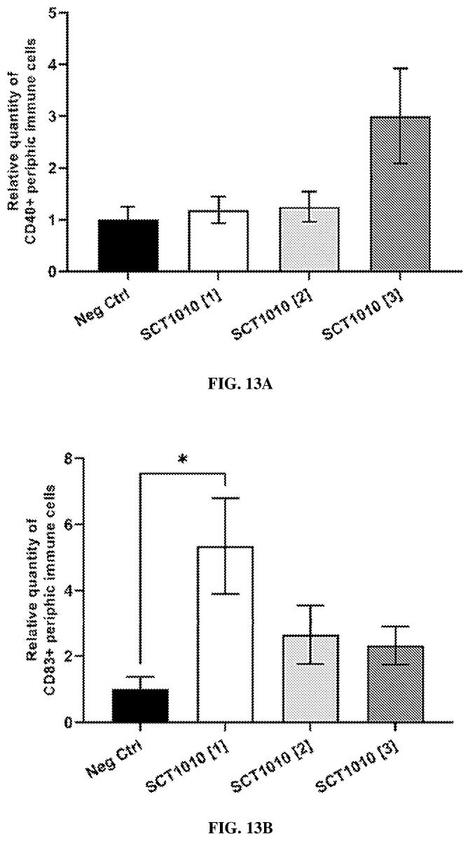

Effects of STC-1010 on the MLR response through IFNγ quantification mDCs co-cultured with autologous CD8⁺ T cells displayed an effective MLR response when previously exposed to CD40L during the maturation period. This effect was evidenced through the evaluation of IFNγ release in the supernatant collected from the co-culture, which showed a significant up-regulation compared to control and vehicle (FIG. 12).

### Conclusion

In the light of these data, STC-1010 has demonstrated its ability to modulate DC maturation as evidenced through the cytokine profile, with an immunogenic and ratio-dependent activity of STC-1010.

This immunogenic profile was confirmed through MLR where CD8⁺ T cells co-cultured with STC-1010-primed DCs had an improved functional activity.

### Example 7

### In Ovo Evaluation of the Functional Activity of STC-1010 Through Chorioallantoic Membrane Assay

The aim was to assess in ovo the final product (DP or “STC-1010”), for its potential to induce an immune response in a chorioallantoic membrane (CAM) assay.

### Materials and Methods

### Preparation of Chicken Embryos

Fertilized White Leghorn eggs were incubated at 37.5° C. with 50% relative humidity for 9 days. At that moment (E9), the CAM was dropped down by drilling a small hole through the eggshell into the air sac, and a 1-cm² window was cut in the eggshell above the CAM.

At least 20 eggs were opened for each study group (but because eggshell opening is an invasive surgical act, some death can occur during the first hours after opening, hence data may have been collected with 15-20 eggs per group).

### Treatment

Before treatment, the viability of each egg was checked and surviving eggs were randomized in groups. All eggs of a group were treated with a volume of 100 μL of STC-1010, with three test conditions:
test condition “STC1010 [1]”: 10⁵ cells/mL, i.e., 10⁴ cells/embryo;
test condition “STC1010 [2]”: 5×10⁵ cells/mL, i.e., 5×10⁴ cells/embryo; and
test condition “STC1010 [3]”: 10⁶ cells/mL, i.e., 10⁵ cells/embryo.

A negative control (“Neg Ctrl”) was performed in parallel, in absence of STC-1010.

### Quantitative Evaluation of Chicken Immune Cells

On day E18, peripheral blood was collected and treated with heparin to prevent blood clotting.

For dendritic cells (DCs) evaluation, 100 μL of individual samples (n=8 per group) was recovered, from which RNA was extracted, reverse-transcribed, pre-amplified and analyzed by qPCR with specific primers for chicken CD40, CD83 and CD86 sequences.

For all points done in qPCR, expression of chicken GAPDH was also analyzed, as reference gene expression, and used to normalize immune biomarker expression between samples. Calculation of C_{q} for each sample, mean C_{q} and relative amounts of immune cells for each group were directly managed by the Bio-Rad® CFX Maestro software.

For T lymphocytes evaluation, the remaining blood samples were pooled within group. Then, blood samples were processed with Hypaque-Ficoll (HF) separation for peripheral blood mononuclear cells (PBMCs) isolation. After that, purified PBMCs were labelled with anti-chicken CD45 (Thermofisher, Ref.: MA5-28679), anti-chicken CD3 (Southern Biotech, Ref.: 8200-26), anti-chicken CD4 (Thermo Fisher, Ref.: MA5-28686) and anti-chicken CD8 (Thermo Fisher, Ref.: MA5-28686) for T lymphocytes evaluation, through flow cytometry analysis.

### Quantitative Evaluation of Immune Cytokines

On day E18, peripheral blood was individually collected (n=5 per group) and treated with heparin to prevent blood clotting. Then, blood samples were centrifuged for plasma collection, which was followed by ELISA analysis for IL-12 and IFNγ expression, each plasma sample being evaluated at three dilutions. All ELISA kits were ordered from Cusabio (chicken IL-12 Elisa kit, Ref.: CSB-E12836C; chicken IFN ELISA kit, Ref.: CSB-E08550Ch).

### Statistical Analysis and Significance

For all quantitative data, the outlier identification and the one-way ANOVA (with post-tests between each couple of groups) were done using Prism® (GraphPad Software).

### Results

### Quantitative Evaluation of Chicken Immune Cells by FACS

Leukocyte activation was evaluated in groups “Neg Ctrl” and “STC-1010 [3]” by FACS quantification of CD45⁺, CD3⁺, CD4⁺ and CD8⁺ cells in PBMCs purified at E18. Table 34 shows the FACS analysis data of these different cell subsets included in peripheric leukocytes (as a % in peripheric CD45⁺ leucocytes).

TABLE 34

| | CD3/CD4 staining | | | |
|---|---|---|---|---|
| | CD3⁻/CD4⁻ | CD3⁺/CD4⁻ | CD3⁻/CD4⁺ | CD3⁺/CD4⁺ |
| Neg Ctrl | 97.5 | 0.6 | 1.7 | 0.3 |
| STC-1010 [3] | 86.4 (↓) | 0.3 (↓) | 12.5 (↑) | 0.5 (↑) |
| | CD3/CD8 staining | | | |
| | CD3⁻/CD8⁻ | CD3⁺/CD8⁻ | CD3⁻/CD8⁺ | CD3⁺/CD8⁺ |
| Neg Ctrl | 98.5 | 0.4 | 0.8 | 0.3 |
| STC-1010 [3] | 97.6 (↓) | 0.1 (↓) | 2.0 (↑) | 0.3 (=) |

These data show an increase of CD4⁺ and CD8⁺ leukocytes, in particular a high increase of CD3⁻/CD4⁺ leukocytes.

### Quantitative Evaluation of Immune Cytokines

IL-12 and IFNγ expression level in peripheric blood was estimated in all groups at E18. To pertinently evaluate cytokines secretion, each plasma sample was evaluated at three dilution ½ ½, 1/10 and 1/50.

Table 35 shows the data analysis of peripheric IL12 secretion.

TABLE 35

| IL-12 analysis | | | | | |
|---|---|---|---|---|---|
| | C° | % | p-value versus STC-1010 [. . .] | | |
| n | (pg/mL) | increase | [1] | [2] | [3] |

Dilution 1/21/2

| | | | | | | |
|---|---|---|---|---|---|---|
| Neg Ctrl | 5 | 1.54 | n.a. | n.a. | n.a. | n.a. |
| STC-1010 [1] | 5 | 2.3 | 49.94 | 0.6896 | n.a. | n.a. |
| STC-1010 [2] | 5 | 2.88 | 87.41 | 0.2510 | 0.8387 | n.a. |
| STC-1010 [3] | 5 | 3.99 | 159.84 | 0.0128 | 0.1093 | 0.4021 |

Dilution at 1/10

| | | | | | | |
|---|---|---|---|---|---|---|
| Neg Ctrl | 5 | 3.24 | n.a. | n.a. | n.a. | n.a. |
| STC-1010 [1] | 5 | 6.63 | 104.74 | 0.4535 | n.a. | n.a. |
| STC-1010 [2] | 5 | 8.23 | 154.23 | 0.1582 | 0.8899 | n.a. |
| STC-1010 [3] | 5 | 17.1 | 427.99 | <0.0001 | 0.0013 | 0.0056 |

Dilution at 1/50

| | | | | | | |
|---|---|---|---|---|---|---|
| Neg Ctrl | 5 | 9.48 | n.a. | n.a. | n.a. | n.a. |
| STC-1010 [1] | 5 | 27 | 184.81 | 0.2099 | n.a. | n.a. |
| STC-1010 [2] | 5 | 19.18 | 102.36 | 0.6723 | 0.7964 | n.a. |
| STC-1010 [3] | 5 | 56.93 | 500.55 | 0.0002 | 0.0139 | 0.0022 |

As seen in these results, a net increase of IL-12 secretion induced by STC-1010 was observed when compared to the negative control, at all three sample dilutions. No significant increase of IFNγ was however observed following STC-1010 treatment (data not shown).

### Quantitative Evaluation of Chicken Immune Cells by RT-gPCR

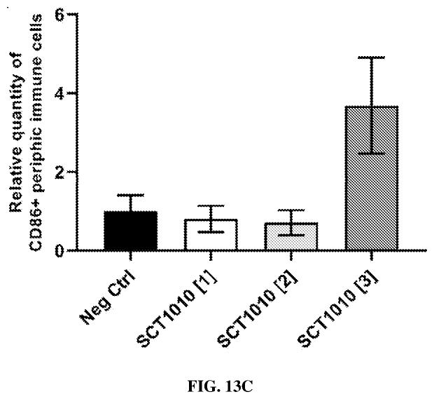

The expression of three DCs activation markers (CD40, CD83, CD86) was evaluated by RT-qPCR. Results show an upregulation of these markers induced by STC-1010, in particular at the highest dose for CD40 and CD86, and at all doses and more significantly at the lowest dose for CD83 (FIG. 13A-C).

Conclusion

Out of the embryos, the vast majority survived throughout this study, confirming the absence of toxicity of STC-1010:
negative control: n=50, 3 embryos died (6% lethality);
group “STC1010 [1]”: n=48, 2 embryos died (4.17% lethality);
group “STC1010 [2]”: n=33, 0 embryo died (0% lethality);
group “STC1010 [3]”: n=29, 1 embryo died (3.45% lethality).

STC-1010 was shown to be able to boost the immune system and activate innate and/or adaptive immune responses using this CAM assay.

Based on these promising results, a new study will be performed to evaluate the in ovo efficacy of STC-1010 on human colorectal adenocarcinoma-grafted chicken embryos.

1-17. (canceled)

18. A composition comprising (i) stressed HT-29, HCT-116 and LoVo cells, and (ii) immunogenic stress proteins produced by these cells in response to a stress applied in vitro.

19. The composition according to claim 18, wherein stressed HT-29, HCT-116 and LoVo cells have developed resistance mechanism in response to one or several stress[es] applied in vitro, selected from the group comprising radiations, thermal stress, chemical stress, metabolic stress and any combinations thereof, leading to the production of the stress proteins.

20. The composition according to claim 18, wherein stressed HT-29, HCT-116 and LoVo cells are non-proliferative.

21. The composition according to claim 18, wherein immunogenic stress proteins are haptenated.

22. The composition according to claim 18, wherein immunogenic stress proteins are haptenated with a hapten selected from the group comprising 2,4-dinitrophenyl (DNP); 2,4-dinitrofluorobenzene; sulfanilic acid; N-iodoacetyl-N′-(5-sulfonic-naphthyl)ethylene diamine; anilin; p-amino benzoic acid; biotin; fluorescein and derivatives thereof (including FITC, TAMRA, and Texas Red); digoxigenin; 5-nitro-3-pyrazolecarbamide; 4,5-dimethoxy-2-nitrocinnamide; 2-(3,4-dimethoxyphenyl)-quinoline-4-carbamide; 2,1,3-benzoxadiazole-5-carbamide; 3-hydroxy-2-quinoxalinecarbamide, 4-(dimethylamino)azobenzene-4′-sulfonamide (DABSYL); rotenone isooxazolinI(E)-2-(2-(2-oxo-2,3-dihydro-1H-benzo[b][1,4]diazepin-4-yl)phenozy)acetamide; 7-(diethylamino)-2-oxo-2H-chromene-3-carboxylic acid; 2-acetamido-4-methyl-5-thiazolesulfonamide; and p-methoxyphenylpyrazopodophyllamide.

23. The composition according to claim 18, being a pharmaceutical composition or a vaccine composition, and further comprising at least one pharmaceutically acceptable excipient.

24. The composition according to claim 18, comprising from about 10⁵ to about 10⁸ stressed HT-29, HCT-116 and LoVo cells.

25. A method of treating cancer in a subject in need thereof, comprising administering to the subject the composition according to claim 18.

26. An intermediate composition comprising (i) one of stressed HT-29 cells, stressed HCT-116 cells and stressed LoVo cells, and (ii) stress proteins,
wherein the one of stressed HT-29 cells, stressed HCT-116 cells or stressed LoVo cells have developed resistance mechanism in response to (i) a metabolic stress, (ii) radiations and (iii) a thermal stress applied in vitro, leading to the production of the stress proteins, or
wherein the one of stressed HT-29 cells, stressed HCT-116 cells or stressed LoVo cells have developed resistance mechanism in response to (i) a metabolic stress, and (ii) a chemical stress applied in vitro, leading to the production of the stress proteins.

27. A method of manufacturing the intermediate compositions according to claim 26 comprising the following steps:
a) cultivating HT-29, HCT-116 or LoVo cells in a suitable culture medium;
b) subjecting the HT-29, HCT-116 or LoVo cells cultured in step a) to one or several stress[es] in vitro, wherein these HT-29, HCT-116 or LoVo cells develop resistance mechanisms in response to the one or several stress[es] and thereby produce stress proteins,
c) recovering the stressed HT-29, HCT-116 or LoVo cells together with the stress proteins they have produced in step b), and
d) treating the stressed HT-29, HCT-116 or LoVo cells and the stress proteins they have produced, all together recovered in step c), with a molecule or by a process capable of rendering the stress proteins immunogenic.

28. The method according to claim 27, wherein step c) is carried out at least several hours after completion of step b).

29. The method according to claim 27, wherein step d) comprises linking the stress proteins to or complexing the stress proteins with a means capable to confer immunogenicity.

30. The method according to claim 29, wherein the means capable to confer immunogenicity is an hapten.

31. The method according to claim 30, wherein the hapten is selected from the group comprising 2,4-dinitrophenyl (DNP); 2,4-dinitrofluorobenzene; sulfanilic acid; N-iodoacetyl-N′-(5-sulfonic-naphthyl)ethylene diamine; anilin; p-amino benzoic acid; biotin; fluorescein and derivatives thereof (including FITC, TAMRA, and Texas Red); digoxigenin; 5-nitro-3-pyrazolecarbamide; 4,5-dimethoxy-2-nitrocinnamide; 2-(3,4-dimethoxyphenyl)-quinoline-4-carbamide; 2,1,3-benzoxadiazole-5-carbamide; 3-hydroxy-2-quinoxalinecarbamide, 4-(dimethylamino)azobenzene-4′-sulfonamide (DABSYL); rotenone isooxazoline; (E)-2-(2-(2-oxo-2,3-dihydro-1H-benzo[b][1,4]diazepin-4-yl)phenozy)acetamide; 7-(diethylamino)-2-oxo-2H-chromene-3-carboxylic acid; 2-acetamido-4-methyl-5-thiazolesulfonamide; and p-methoxyphenylpyrazopodophyllamide.

32. The method according to claim 27, wherein step b) comprises subjecting the HT-29, HCT-116 or LoVo cells cultured in step a) to the following stresses in vitro, applied concomitantly or successively:
(i) an in vitro culture in a depleted medium, under hypoxia, and/or at low pH;
(ii) an in vitro radiation with a total dose ranging from about 0.25 to about 25 Gy, for a period ranging from about 1 to about 20 minutes, and
(iii) an in vitro thermic choc at a temperature ranging from about 38° C. to about 45° C., applied to the cells for a period ranging from about 15 minutes to about 4 hours.

33. The method according to claim 27, wherein step b) comprises subjecting the HT-29, HCT-116 or LoVo cells cultured in step a) to the following stresses in vitro, applied concomitantly or successively:
(i) an in vitro culture in a depleted medium, under hypoxia, and/or at low pH,
(ii) an in vitro exposition to at least one or several chemotherapeutic agents and/or alcohols, for a period ranging from about 6 hours to about 120 hours.

34. The method according to claim 33, wherein:
the cells are HT-29 cells and the in vitro exposition at (ii) is to about 13 μM oxaliplatin for a period of about 72 hours; or
the cells are HCT-116 cells and the in vitro exposition at (ii) is to about 315 nM SN-38 (7-ethyl-10-hydroxy-camptothecin) for a period of about 48 hours; or
the cells are LoVo cells and the in vitro exposition at (ii) is to about 5 μM fluorouracil (5-FU) for a period of about 48 hours.

35. A method of manufacturing the composition according to claim 18, comprising the following steps:
a) obtaining six intermediate compositions,
wherein the six intermediate compositions are:
1) an intermediate composition comprising stressed HT-29 cells and stress proteins, wherein the stressed HT-29 cells have developed resistance mechanism in response to (i) a metabolic stress, (ii) radiations and (iii) a thermal stress applied in vitro, leading to the production of the stress proteins,
2) an intermediate composition comprising stressed HCT-116 cells and stress proteins, wherein the stressed HCT-116 cells have developed resistance mechanism in response to (i) a metabolic stress, (ii) radiations and (iii) a thermal stress applied in vitro, leading to the production of the stress proteins,
3) an intermediate composition comprising stressed LoVo cells and stress proteins, wherein the stressed LoVo cells have developed resistance mechanism in response to (i) a metabolic stress, (ii) radiations and (iii) a thermal stress applied in vitro, leading to the production of the stress proteins,
4) an intermediate composition comprising stressed HT-29 cells and stress proteins, wherein the stressed HT-29 cells have developed resistance mechanism in response to (i) a metabolic stress, and (ii) a chemical stress applied in vitro, leading to the production of the stress proteins,
5) an intermediate composition comprising stressed HCT-116 cells and stress proteins, wherein the stressed HCT-116 cells have developed resistance mechanism in response to (i) a metabolic stress, and (ii) a chemical stress applied in vitro, leading to the production of the stress proteins,
6) an intermediate composition comprising stressed LoVo cells and stress proteins, wherein the stressed LoVo cells have developed resistance mechanism in response to (i) a metabolic stress, and (ii) a chemical stress applied in vitro, leading to the production of the stress proteins,
b) mixing these six intermediate compositions together.

36. The method according to claim 35, wherein the six intermediate compositions are mixed together in an equal ratio of stressed HT-29, HCT-116 and LoVo cells.
